(19) United States
(12) Patent Application Publication
(10) Pub. No.: US 2025/0346657 A1
(43) Pub. Date: Nov. 13, 2025

(54) CANINIZED AND FELINIZED ANTIBODIES TO HUMAN NGF

(71) Applicant: INTERVET INC., Madison, NJ (US)

(72) Inventors: Mohamad MORSEY, Omaha, NE (US); Yuanzheng ZHANG, Somerset, NJ (US)

(73) Assignee: INTERVET INC., Madison, NJ (US)

(21) Appl. No.: 18/720,586

(22) PCT Filed: Dec. 15, 2022
(86) PCT No.: PCT/EP2022/086097

§ 371 Date: Jun. 14, 2024

Related U.S. Application Data
(60) Provisional application No. 63/290,264, filed on Dec. 16, 2021.
(60) Provisional application No. 63/327,076, filed on Apr. 4, 2022.

(51) Int. Cl. C07K 16/22; A61P 25/02

(52) U.S. Cl. CPC C07K 16/22; A61P 25/02; C07K 2317/24; C07K 2317/52; C07K 2317/565; C07K 2317/73; C07K 2317/92

(57) ABSTRACT

The present invention provides caninized and felinized anti-human NGF antibodies that have a high binding affinity for canine NGF. The invention also relates to use of these antibodies in the treatment of pain in canines, felines, and other companion animals.

### CROSS-REFERENCE TO RELATED APPLICATIONS

This application is a U.S. National Stage Application under 35 U.S.C. § 371 of International Patent Application No. PCT/EP2022/086097, filed on Dec. 15, 2022, which claims the benefit of U.S. Provisional Application No. 63/327,076, filed on Apr. 4, 2022, and U.S. Provisional Application No. 63/290,264, filed on Dec. 16, 2021, the disclosure of each of which is incorporated by reference herein in its entirety.

### REFERENCE TO SEQUENCE LISTING SUBMITTED ELECTRONICALLY

This application contains an electronic Substitute Sequence Listing which has been submitted in XML format via Patent Center, the entire content of which is incorporated by reference herein in its entirety. The Substitute Sequence Listing XML file submitted via Patent Center is entitled “14463-301-999_SUB_SEQ_LISTING.xml”, was created on Jan. 3, 2025, and is 92,344 bytes in size.

### FIELD OF THE INVENTION

The present invention relates to antibodies to proteins involved in pain. More particularly, the present invention further relates to caninized or felinized antibodies to human NGF that have a high binding affinity for canine and feline NGF. The present invention also relates to use of the antibodies of the present invention in the treatment of pain in canines and felines with osteoarthritis.

### BACKGROUND OF THE INVENTION

Nerve growth factor (NGF) is a well-characterized secreted protein that plays an important role in the development of the nervous system. In addition, NGF has also been shown to have biological effects on non-neuronal cells and tissues including cells of the immune system. NGF initially was isolated in the mouse submandibular gland as a complex composed of three non-covalently linked subunits. The alpha and gamma subunits of NGF belong to the kallikrein family of serine proteases, whereas the beta subunit of NGF complex exhibits the biological activities attributed to NGF. NGF (also referred to as Beta NGF) is produced as a prepropeptide with 18-amino acid residue signal peptide [Wiesmann and de Vos, CLMS: 58, 748-759, (2001)]. Recombinant human beta-NGF is a homodimer of two 120 amino acid polypeptides. The C-terminal 120 amino acids of human NGF has approximately 98% homology to the predicted C-terminal end of NGF from other species, including canines and felines.

A number of studies indicate that NGF plays a key role in the transmission of pain. For example, in humans, NGF levels are elevated in the synovial fluids from patients with some arthritic conditions [Aloe, et al., Arch. Rheum., 35:351-355 (1992)] Moreover, elevated levels of canine NGF expression have been demonstrated in synovial fluids of dogs with osteoarthritis [Isola, et al., Vet Comp. Orthop. Traumatol., 4:279 (2011)] It also has been demonstrated that agents that inhibit the function of NGF such as neutralizing antibodies prevent hyperalgesia and allodynia in animal models of neuropathic pain [see, e.g., Ramer et al., Eur. J. Neurosci. 11:837-846 (1999) and Ro et al., Pain, 79 265-274 (1999)]. The realization that NGF is involved in the transmission of pain in certain inflammatory and non-inflammatory conditions such as osteoarthritis and cancer led to interest in developing antibodies that can neutralize the biological activities of NGF. [Examples of anti-NGF antibodies known in the art include WO01/78698, WO 01/64247, WO 02/096458, U.S. Pat. No. 7,601,818 B2, and Gearing et al., BMC Veterinary Research, 9:226, (2013)].

The citation of any reference herein should not be construed as an admission that such reference is available as “prior art” to the instant application.

### SUMMARY OF THE INVENTION

The present invention relates to caninized and felinized anti-human nerve growth factor (NGF) antibodies that have specific binding affinity for canine and/or feline NGF, as well as having the ability to block the binding of canine and/or feline NGF to the respective NGF receptor. The present invention includes the use of such antibodies in the treatment of hyperalgesia and allodynia in animal. The antibodies also can be used to treat pain in canines (dogs) or felines (cats) with osteoarthritis.

Accordingly, the present invention provides novel caninized and felinized antibodies that comprise a heavy chain and a light chain, and antigen binding fragments thereof of the caninized and felinized antibodies, that are capable of binding and neutralizing NGF. The heavy chain of the caninized antibody comprises a variable region (VH) and three constant regions, which includes the canine fragment crystallizable region (cFc or cFc region). The light chain also comprises a variable region (VL), but just one constant region. The respective variable regions of the heavy chain and light chain each comprise three hypervariable regions, i.e., complementary determining regions (CDRs). Therefore, the light chain comprises three light chain complementary determining regions (CDRs): CDR light 1 (CDRL1), CDR light 2 (CDRL2), and CDR light 3 (CDRL3) each comprising an amino acid sequence, whereas the heavy chain comprises three heavy chain CDRs: CDR heavy 1 (CDRH1), CDR heavy 2 (CDRH2) and CDR heavy 3 (CDRH3) each comprising an amino acid sequence. The CDRH1 comprises the amino acid sequence of SEQ ID NO: 1, the CDRH2 comprises the amino acid sequence of SEQ ID NO: 2, and the CDRH3 comprises the amino acid sequence of SEQ ID NO: 3, whereas CDRL1 comprises the amino acid sequence of SEQ ID NO: 4, the CDRL2 comprises the amino acid sequence of SEQ ID NO: 5, and the CDRL3 comprises the amino acid sequence of SEQ ID NO: 6.

In certain embodiments, the caninized antibody comprises a heavy chain variable region that comprises the amino acid sequence of SEQ ID NO: 27. In other embodiments, the caninized antibody comprises a heavy chain variable region that comprises the amino acid sequence of SEQ ID NO: 28. In related embodiments, the caninized antibody comprises a light chain variable region that comprises the amino acid sequence of SEQ ID NO: 29. In other related embodiments, the caninized antibody comprises a light chain variable region that comprises the amino acid sequence of SEQ ID NO: 30. The present invention further provides antigen binding fragments of all of these antibodies.

In particular embodiments, the caninized antibody comprises a heavy chain variable region that comprises the amino acid sequence of SEQ ID NO: 27 and the caninized antibody comprises a light chain variable region that comprises the amino acid sequence of SEQ ID NO: 29. In other embodiments, the caninized antibody comprises a heavy chain variable region that comprises the amino acid sequence of SEQ ID NO: 27 and the caninized antibody comprises a light chain variable region that comprises the amino acid sequence of SEQ ID NO: 30. In yet other embodiments, the caninized antibody comprises a heavy chain variable region that comprises the amino acid sequence of SEQ ID NO: 28 and the caninized antibody comprises a light chain variable region that comprises the amino acid sequence of SEQ ID NO: 29. In still other embodiments, the caninized antibody comprises a heavy chain variable region that comprises the amino acid sequence of SEQ ID NO: 28 and the caninized antibody comprises a light chain variable region that comprises the amino acid sequence of SEQ ID NO: 30. The present invention further provides antigen binding fragments of all of these antibodies.

The caninized antibodies also comprise a hinge region. The canine hinge region is preferably a canine hinge region. In certain embodiments, the canine hinge region comprises an amino acid sequence that comprises at least 90%, 95%, or 100% identity with the amino acid sequence of SEQ ID NO: 45. In other embodiments, the canine hinge region comprises an amino acid sequence that comprises at least 90%, 95%, or 100% identity with the amino acid sequence of SEQ ID NO: 46. In yet other embodiments, the canine hinge region comprises an amino acid sequence that comprises at least 90%, 95%, or 100% identity with the amino acid sequence of SEQ ID NO: 47. In still other embodiments, the canine hinge region comprises an amino acid sequence that comprises at least 90%, 95%, or 100% identity with the amino acid sequence of SEQ ID NO: 48. The present invention further provides antigen binding fragments of all of these antibodies.

The caninized antibodies of the present invention comprise a canine fragment crystallizable region (cFc region). In one embodiment, the canine cFc region comprises an amino acid sequence that comprises at least 90%, 95%, 98%, 99%, or 100% identity with the amino acid sequence of SEQ ID NO: 49. In another embodiment, the canine cFc region comprises an amino acid sequence that comprises at least 90%, 95%, 98%, 99%, or 1⁰⁰% identity with the amino acid sequence of SEQ ID NO: 50. In yet another embodiment, the canine cFc region comprises an amino acid sequence that comprises at least 90%, 95%, 98%, 99%, or 100% identity with the amino acid sequence of SEQ ID NO: 52. In still another embodiment, the canine cFc region comprises an amino acid sequence that comprises at least 90%, 95%, 98%, 99%, or 100% identity with the amino acid sequence of SEQ ID NO: 53. In yet another embodiment, the canine cFc region is a cFc IgG-Bm that comprises an amino acid sequence that comprises at least 90%, 95%, 98%, 99%, or 100% identity with the amino acid sequence of SEQ ID NO: 20 and/or SEQ ID NO: 51, in which both the aspartic acid residue (D) at position 31 of SEQ ID NO: 50 and the asparagine residue (N) at position 63 of SEQ ID NO: 50, are substituted by an alanine residue (A). The present invention further provides antigen binding fragments of all of these antibodies.

In some embodiments, the caninized antibody comprises a light chain that comprises the amino acid sequence of SEQ ID NO: 38. In other embodiments, the caninized antibody comprises a light chain that comprises the amino acid sequence of SEQ ID NO: 39. In still other embodiments, the caninized antibody comprises a heavy chain that comprises the amino acid sequence of SEQ ID NO: 36. In yet other embodiments, the caninized antibody comprises a heavy chain that comprises the amino acid sequence of SEQ ID NO: 37. The present invention further provides antigen binding fragments of all of these antibodies.

In particular embodiments, the caninized antibody comprises a heavy chain that comprises the amino acid sequence of SEQ ID NO: 36 and comprises a light chain that comprises the amino acid sequence of SEQ ID NO: 38. In other embodiments, the caninized antibody comprises a heavy chain that comprises the amino acid sequence of SEQ ID NO: 36 and comprises a light chain that comprises the amino acid sequence of SEQ ID NO: 39. In still other embodiments, the caninized antibody comprises a heavy chain that comprises the amino acid sequence of SEQ ID NO: 37 and comprises a light chain that comprises the amino acid sequence of SEQ ID NO: 38. In yet other embodiments, the caninized antibody comprises a heavy chain that comprises the amino acid sequence of SEQ ID NO: 37 and comprises a light chain that comprises the amino acid sequence of SEQ ID NO: 39. The present invention further provides antigen binding fragments of all of these antibodies.

The present invention further provides nucleic acids (including isolated nucleic acids) that encode any one of the light chain variable regions of the caninized antibodies of the present invention. In certain embodiments, the nucleic acid encodes the light chain variable region that comprises the amino acid sequence of SEQ ID NO: 29. In other embodiments, the nucleic acid encodes the light chain variable region that comprises the amino acid sequence of SEQ ID NO: 30.

The present invention also provides nucleic acids that encode any one of the light chains of the caninized antibodies of the present invention. In certain embodiments, the nucleic acid encodes the light chain that comprises the amino acid sequence of SEQ ID NO: 38. In other embodiments, the nucleic acid encodes the light chain that comprises the amino acid sequence of SEQ ID NO: 39.

Similarly, the present invention further provides nucleic acids that encode any one of the heavy chain variable regions of the caninized antibodies of the present invention. In certain embodiments, the nucleic acid encodes the heavy chain variable region that comprises the amino acid sequence of SEQ ID NO: 27. In other embodiments, the nucleic acid encodes the heavy chain variable region that comprises the amino acid sequence of SEQ ID NO: 28.

In addition, the present invention further provides nucleic acids that encode any one of the heavy chains of the caninized antibodies of the present invention. In related embodiments, the nucleic acid encodes a heavy chain that comprises the amino acid sequence of SEQ ID NO: 36. In other embodiments, the nucleic acid encodes a heavy chain that comprises the amino acid sequence of SEQ ID NO: 37. The present invention further provides nucleic acids that encode any one of the antigen binding fragments of the antibodies of the present invention.

Accordingly, the present invention provides a pair of nucleic acids, wherein one of the pair of nucleic acids comprises a nucleotide sequence that encodes the heavy chain of a specific felinized antibody of the present invention and the other of the pair of nucleic acids comprises a nucleotide sequence that encodes the light chain of said specific felinized antibody.

The present invention further provides expression vectors that comprise and express one or more of the nucleic acids of the present invention. In particular embodiments, the expression vector comprises and expresses a nucleic acid encoding a heavy chain of a caninized antibody of the present invention and a nucleic acid encoding a light chain of that caninized antibody. The present invention also provides host cells that comprise one or more expression vectors of the present invention.

The present invention also provides pharmaceutical compositions comprising the caninized antibodies and/or antigen binding fragments of the antibodies and a pharmaceutically acceptable carrier or diluent. In addition, pharmaceutical compositions are provided that comprise a nucleic acid encoding a heavy chain of a caninized antibody of the present invention and a nucleic acid encoding a light chain of that caninized antibody and a pharmaceutically acceptable carrier or diluent. In other embodiments, the pharmaceutical compositions comprise a nucleic acid encoding both a heavy chain of a caninized antibody of the present invention and a light chain of that caninized antibody. In yet other embodiments, the pharmaceutical compositions comprise a pharmaceutically acceptable carrier or diluent and an expression vector that comprises one or more nucleic acids encoding a heavy chain of a caninized antibody of the present invention and a light chain of that caninized antibody and thereby, can express the caninized antibody and/or antigen binding fragments of the antibody of the present invention, in vivo.

The present invention further provides methods of treating a condition associated with pain in an animal subject. The method of treatment can comprise administering to an animal subject in need thereof, a therapeutically effective amount of a pharmaceutical composition of the present invention. In certain embodiments, the method is used for the treatment of osteoarthritis. In other embodiments, the method is used for the treatment of hyperalgesia. In still other embodiments, the method is used for the treatment of allodynia. In yet other embodiments, the method is used for the treatment of pain. In still other embodiments, the method is used for the treatment of any combination of osteoarthritis, hyperalgesia, allodynia, and/or pain. When the antibody administered is a caninized antibody, the animal subject is preferably a canine.

The present invention also provides methods of producing a caninized antibody or antigen binding fragment thereof that binds canine NGF. In particular embodiments, the method includes culturing one or more host cells comprising one or more expression vectors that encode and express the light chain of a caninized antibody of the present invention and/or the heavy chain of that caninized antibody in a culture medium under conditions in which the nucleic acid is expressed, thereby producing a polypeptide comprising the light chain of a caninized antibody of the present invention, and/or the heavy chain of that caninized antibody. The polypeptides are then recovered from the one or more host cells or culture medium. In certain embodiments, the polypeptides comprising the light chain of a caninized antibody of the present invention and the polypeptides comprising the heavy chain of that caninized antibody are combined with each under conditions that are conducive to form a caninized antibody.

As indicated above, the present invention also pertains to felinized anti-human nerve growth factor (NGF) antibodies that have specific binding affinity for canine and/or feline NGF, as well as having the ability to block the binding of canine and/or feline NGF to the respective NGF receptor. Accordingly, the light chain comprises three light chain complementary determining regions (CDRs): CDR light 1 (CDRL1), CDR light 2 (CDRL2), and CDR light 3 (CDRL3) each comprising an amino acid sequence, whereas the heavy chain comprises three heavy chain CDRs: CDR heavy 1 (CDRH1), CDR heavy 2 (CDRH2) and CDR heavy 3 (CDRH3) each comprising an amino acid sequence. The CDRH1 comprises the amino acid sequence of SEQ ID NO: 1, the CDRH2 comprises the amino acid sequence of SEQ ID NO: 2, and the CDRH3 comprises the amino acid sequence of SEQ ID NO: 3, whereas CDRL1 comprises the amino acid sequence of SEQ ID NO: 4, the CDRL2 comprises the amino acid sequence of SEQ ID NO: 5, and the CDRL3 comprises the amino acid sequence of SEQ ID NO: 6.

In certain embodiments, the felinized antibody comprises a heavy chain variable region that comprises the amino acid sequence of SEQ ID NO: 57. In other embodiments, the felinized antibody comprises a heavy chain variable region that comprises the amino acid sequence of SEQ ID NO: 58. In still other embodiments, the felinized antibody comprises a heavy chain variable region that comprises the amino acid sequence of SEQ ID NO: 59. In yet other embodiments, the felinized antibody comprises a heavy chain variable region that comprises the amino acid sequence of SEQ ID NO: 60.

In related embodiments, the felinized antibody comprises a light chain variable region that comprises the amino acid sequence of SEQ ID NO: 61. In other related embodiments, the felinized antibody comprises a light chain variable region that comprises the amino acid sequence of SEQ ID NO: 62. The present invention further provides antigen binding fragments of all of these antibodies.

In particular embodiments, the felinized antibody comprises a heavy chain variable region that comprises the amino acid sequence of SEQ ID NO: 57 and the felinized antibody comprises a light chain variable region that comprises the amino acid sequence of SEQ ID NO: 61. In other embodiments, the felinized antibody comprises a heavy chain variable region that comprises the amino acid sequence of SEQ ID NO: 57 and the felinized antibody comprises a light chain variable region that comprises the amino acid sequence of SEQ ID NO: 62. In yet other embodiments, the felinized antibody comprises a heavy chain variable region that comprises the amino acid sequence of SEQ ID NO: 58 and the felinized antibody comprises a light chain variable region that comprises the amino acid sequence of SEQ ID NO: 61. In still other embodiments, the felinized antibody comprises a heavy chain variable region that comprises the amino acid sequence of SEQ ID NO: 58 and the felinized antibody comprises a light chain variable region that comprises the amino acid sequence of SEQ ID NO: 62. In yet other embodiments the felinized antibody comprises a heavy chain variable region that comprises the amino acid sequence of SEQ ID NO: 59 and the felinized antibody comprises a light chain variable region that comprises the amino acid sequence of SEQ ID NO: 61. In still other embodiments, the felinized antibody comprises a heavy chain variable region that comprises the amino acid sequence of SEQ ID NO: 59 and the felinized antibody comprises a light chain variable region that comprises the amino acid sequence of SEQ ID NO: 62. In yet other embodiments, the felinized antibody comprises a heavy chain variable region that comprises the amino acid sequence of SEQ ID NO: 60 and the felinized antibody comprises a light chain variable region that comprises the amino acid sequence of SEQ ID NO: 61. In still other embodiments, the felinized antibody comprises a heavy chain variable region that comprises the amino acid sequence of SEQ ID NO: 60 and the felinized antibody comprises a light chain variable region that comprises the amino acid sequence of SEQ ID NO: 62. The present invention further provides antigen binding fragments of all of these antibodies.

The felinized antibodies also comprise a hinge region. The feline hinge region is preferably a feline hinge region.

The felinized antibodies of the present invention further comprise a feline heavy chain constant region. In one embodiment, the feline heavy chain constant region comprises an amino acid sequence that comprises at least 90%, 95%, 98%, 99%, or 100% identity with the amino acid sequence of SEQ ID NO: 54. In another embodiment, the feline heavy chain constant region comprises an amino acid sequence that comprises at least 90%, 95%, 98%, 99%, or 100% identity with the amino acid sequence of SEQ ID NO: 55. In yet another embodiment, the feline heavy chain constant region comprises an amino acid sequence that comprises at least 90%, 95%, 98%, 99%, or 100% identity with the amino acid sequence of SEQ ID NO: 56. In still another embodiment, the feline heavy chain constant region comprises an amino acid sequence that comprises at least 90%, 95%, 98%, 99%, or 100% identity with the amino acid sequence of SEQ ID NO: 54, in which both the aspartic acid residue (D) at position 151 and the asparagine residue (N) at position 183, are both substituted by an alanine residue (A). In yet another embodiment, the feline heavy chain constant region comprises an amino acid sequence that comprises at least 90%, 95%, 98%, 99%, or 100% identity with the amino acid sequence of SEQ ID NO: 55, in which both the aspartic acid residue (D) at position 151 and the asparagine residue (N) at position 183, are both substituted by an alanine residue (A). The present invention further provides antigen binding fragments of all of these antibodies.

In some embodiments, the felinized antibody comprises a light chain that comprises the amino acid sequence of SEQ ID NO: 68. In other embodiments, the felinized antibody comprises a light chain that comprises the amino acid sequence of SEQ ID NO: 69. In related embodiments, the felinized antibody comprises a heavy chain that comprises the amino acid sequence of SEQ ID NO: 64. In other embodiments, the felinized antibody comprises a heavy chain that comprises the amino acid sequence of SEQ ID NO: 65. In yet other embodiments, the felinized antibody comprises a heavy chain that comprises the amino acid sequence of SEQ ID NO: 66. In still other embodiments, the felinized antibody comprises a heavy chain that comprises the amino acid sequence of SEQ ID NO: 67. The present invention further provides antigen binding fragments of all of these antibodies.

In particular embodiments, the felinized antibody comprises a heavy chain that comprises the amino acid sequence of SEQ ID NO: 64 and comprises a light chain that comprises the amino acid sequence of SEQ ID NO: 68. In other embodiments, the felinized antibody comprises a heavy chain that comprises the amino acid sequence of SEQ ID NO: 64 and comprises a light chain that comprises the amino acid sequence of SEQ ID NO: 69. In still other embodiments, the felinized antibody comprises a heavy chain that comprises the amino acid sequence of SEQ ID NO: 65 and comprises a light chain that comprises the amino acid sequence of SEQ ID NO: 68. In yet other embodiments, the felinized antibody comprises a heavy chain that comprises the amino acid sequence of SEQ ID NO: 65 and comprises a light chain that comprises the amino acid sequence of SEQ ID NO: 69. In still other embodiments, the felinized antibody comprises a heavy chain that comprises the amino acid sequence of SEQ ID NO: 66 and comprises a light chain that comprises the amino acid sequence of SEQ ID NO: 68. In yet other embodiments, the felinized antibody comprises a heavy chain that comprises the amino acid sequence of SEQ ID NO: 66 and comprises a light chain that comprises the amino acid sequence of SEQ ID NO: 69. In still other embodiments, the felinized antibody comprises a heavy chain that comprises the amino acid sequence of SEQ ID NO: 67 and comprises a light chain that comprises the amino acid sequence of SEQ ID NO: 68. In yet other embodiments, the felinized antibody comprises a heavy chain that comprises the amino acid sequence of SEQ ID NO: 67 and comprises a light chain that comprises the amino acid sequence of SEQ ID NO: 69.

The present invention further provides antigen binding fragments of all of these antibodies.

The present invention also provides nucleic acids, including isolated nucleic acids, that encode any of the felinized antibodies of the present invention and antigen binding fragments thereof. Therefore, the present invention provides nucleic acids (including isolated nucleic acids) that encode any one of the light chain variable regions of the felinized antibodies of the present invention. In certain embodiments, the nucleic acid encodes the light chain variable region that comprises the amino acid sequence of SEQ ID NO: 61. In other embodiments, the nucleic acid encodes the light chain variable region that comprises the amino acid sequence of SEQ ID NO: 62. The present invention also provides nucleic acids that encode any one of the light chains of the felinized antibodies of the present invention. In certain embodiments, the nucleic acid encodes the light chain that comprises the amino acid sequence of SEQ ID NO: 68. In other embodiments, the nucleic acid encodes the light chain that comprises the amino acid sequence of SEQ ID NO: 69.

Similarly, the present invention further provides nucleic acids that encode any one of the heavy chain variable regions of the felinized antibodies of the present invention. In certain embodiments, the nucleic acid encodes the nucleic acid encodes the heavy chain variable region that comprises the amino acid sequence of SEQ ID NO: 57. In other embodiments, the nucleic acid encodes the heavy chain variable region that comprises the amino acid sequence of SEQ ID NO: 58. In still other embodiments, the nucleic acid encodes the heavy chain variable region that comprises the amino acid sequence of SEQ ID NO: 59. In yet other embodiments, the nucleic acid encodes the heavy chain variable region that comprises the amino acid sequence of SEQ ID NO: 60.

In addition, the present invention further provides nucleic acids that encode any one of the heavy chains of the felinized antibodies of the present invention. In certain embodiments, the nucleic acid encodes a heavy chain that comprises the amino acid sequence of SEQ ID NO: 64. In other embodiments, the nucleic acid encodes a heavy chain that comprises the amino acid sequence of SEQ ID NO: 65. In related embodiments, the nucleic acid encodes a heavy chain that comprises the amino acid sequence of SEQ ID NO: 66. In other embodiments, the nucleic acid encodes a heavy chain that comprises the amino acid sequence of SEQ ID NO: 67.

The present invention further provides nucleic acids that encode any one of the antigen binding fragments of the antibodies of the present invention.

Accordingly, the present invention provides nucleic acids encoding the heavy chain variable regions of the felinized antibodies or antigen binding fragments thereof; the heavy chains of the felinized antibodies or antigen binding fragments thereof, the light chain variable regions of the felinized antibodies or antigen binding fragments thereof, and/or the light chains of the felinized antibodies or antigen binding fragments thereof. The present invention further provides a pair of nucleic acids, wherein one of the pair of nucleic acids comprises a nucleotide sequence that encodes the light chain of a specific felinized antibody of any one of the antibodies of the present invention and the other of the pair of nucleic acids comprises a nucleotide sequence that encodes the heavy chain of that (said) specific felinized antibody. The present invention also provides expression vectors that comprise such pairs of nucleic acids, or alternatively individual nucleic acids of the present invention. In addition, the present invention provides pairs of expression vectors, wherein one of the pair of expression vectors comprises a nucleic acid comprising a nucleotide sequence that encodes the light chain of a specific felinized antibody of any one of the felinized antibodies of the present invention, and the other of the pair of expression vectors comprises a nucleic acid comprising a nucleotide sequence that encodes the heavy chain of that (said) specific felinized antibody. Therefore, the present invention provides nucleic acids that encode the heavy chain variable region of a felinized antibody or an antigen binding fragment thereof of the present invention. The present invention further provides nucleic acids that encode the heavy chain of a felinized antibody or an antigen binding fragment thereof of the present invention. The present invention also provides nucleic acids that encode the light chain variable region of a felinized antibody or an antigen binding fragment thereof of the present invention. The present invention also provides nucleic acids that encode the light chain of a felinized antibody or an antigen binding fragment thereof of the present invention. In certain embodiments, the nucleic acid encoding the heavy chain variable region encodes the heavy chain of a felinized antibody and the corresponding nucleic acid encoding the light chain variable region encodes the light chain of that felinized antibody.

The present invention further provides as a pair, a nucleic acid encoding a set of the three heavy chain CDRs and a nucleic acid that encodes the corresponding set of the three light chain CDRs. In certain embodiments, the nucleic acid encoding the set of the three heavy chain CDRs encodes the heavy chain variable region of a felinized antibody and the corresponding nucleic acid encoding the set of the three light chain CDRs encodes the light chain variable region of that (said) felinized antibody. The present invention also provides a kit containing this pair of two nucleic acids. In certain embodiments, a nucleic acid encoding the set of the three heavy chain CDRs encodes the heavy chain of a felinized antibody and the corresponding nucleic acid encoding the set of the three light chain CDRs encodes the light chain of that felinized antibody.

In particular embodiments, a nucleic acid encodes a felinized antibody heavy chain that comprises a CDRH1 comprising the amino acid sequence of SEQ ID NO: 1, a CDRH2 comprising the amino acid sequence of SEQ ID NO: 2, and a CDRH3 comprising the amino acid sequence of SEQ ID NO: 3. In a related embodiments, a nucleic acid encodes a felinized antibody light chain that comprises a CDRL1 comprising the amino acid sequence of SEQ ID NO: 4, a CDRL2 comprising the amino acid sequence of SEQ ID NO: 5, and a CDRL3 comprising the amino acid sequence of SEQ ID NO: 6. The present invention further provides as a pair, a nucleic acid encoding a felinized antibody heavy chain that comprises a CDRH1 comprising the amino acid sequence of SEQ ID NO: 1, a CDRH2 comprising the amino acid sequence of SEQ ID NO: 2, and a CDRH3 comprising the amino acid sequence of SEQ ID NO: 3 and a nucleic acid encoding a felinized antibody light chain that comprises a CDRL1 comprising the amino acid sequence of SEQ ID NO: 4, a CDRL2 comprising the amino acid sequence of SEQ ID NO: 5, and a CDRL3 comprising the amino acid sequence of SEQ ID NO: 6. The present invention also provides a kit containing this pair of two nucleic acids.

In specific embodiments, a nucleic acid of the present invention encodes a heavy chain variable region of a felinized antibody or antigen binding fragment thereof in which the heavy chain variable region comprises the amino acid sequence of SEQ ID NO: 57. In a related embodiment, a nucleic acid encodes the light chain variable region of the felinized antibody or antigen binding fragment thereof in which the light chain variable region comprises the amino acid sequence of SEQ ID NO: 61. The present invention further provides a pair of nucleic acids, wherein one of the pair of nucleic acids comprises a nucleotide sequence that encodes the heavy chain variable region that comprises the amino acid sequence of SEQ ID NO: 57 and the other of the pair of nucleic acids comprises a nucleotide sequence that encodes the light chain variable region that comprises the amino acid sequence of SEQ ID NO: 61. The present invention also provides a kit containing this pair of two nucleic acids. In certain embodiments, the nucleic acid encoding the heavy chain variable region encodes the heavy chain of a felinized antibody and the corresponding nucleic acid encoding the light chain variable region encodes the light chain of that felinized antibody.

In other specific embodiments, a nucleic acid of the present invention encodes a heavy chain variable region of a felinized antibody or antigen binding fragment thereof in which the heavy chain variable region comprises the amino acid sequence of SEQ ID NO: 57. In a related embodiment, a nucleic acid encodes the light chain variable region of the felinized antibody or antigen binding fragment thereof in which the light chain variable region comprises the amino acid sequence of SEQ ID NO: 62. The present invention further provides a pair of nucleic acids, wherein one of the pair of nucleic acids comprises a nucleotide sequence that encodes the heavy chain variable region that comprises the amino acid sequence of SEQ ID NO: 57 and the other of the pair of nucleic acids comprises a nucleotide sequence that encodes the light chain variable region that comprises the amino acid sequence of SEQ ID NO: 62. The present invention also provides a kit containing this pair of two nucleic acids. In certain embodiments, the nucleic acid encoding the heavy chain variable region encodes the heavy chain of a felinized antibody and the corresponding nucleic acid encoding the light chain variable region encodes the light chain of that felinized antibody.

In still other specific embodiments, a nucleic acid of the present invention encodes a heavy chain variable region of a felinized antibody or antigen binding fragment thereof in which the heavy chain variable region comprises the amino acid sequence of SEQ ID NO: 58. In a related embodiment, a nucleic acid encodes the light chain variable region of the felinized antibody or antigen binding fragment thereof in which the light chain variable region comprises the amino acid sequence of SEQ ID NO: 61. The present invention further provides a pair of nucleic acids, wherein one of the pair of nucleic acids comprises a nucleotide sequence that encodes the heavy chain variable region that comprises the amino acid sequence of SEQ ID NO: 58 and the other of the pair of nucleic acids comprises a nucleotide sequence that encodes the light chain variable region that comprises the amino acid sequence of SEQ ID NO: 61. The present invention also provides a kit containing this pair of two nucleic acids. In certain embodiments, the nucleic acid encoding the heavy chain variable region encodes the heavy chain of a felinized antibody and the corresponding nucleic acid encoding the light chain variable region encodes the light chain of that felinized antibody.

In yet other specific embodiments, a nucleic acid of the present invention encodes a heavy chain variable region of a felinized antibody or antigen binding fragment thereof in which the heavy chain variable region comprises the amino acid sequence of SEQ ID NO: 58. In a related embodiment, a nucleic acid encodes the light chain variable region of the felinized antibody or antigen binding fragment thereof in which the light chain variable region comprises the amino acid sequence of SEQ ID NO: 62. The present invention further provides a pair of nucleic acids, wherein one of the pair of nucleic acids comprises a nucleotide sequence that encodes the heavy chain variable region that comprises the amino acid sequence of SEQ ID NO: 58 and the other of the pair of nucleic acids comprises a nucleotide sequence that encodes the light chain variable region that comprises the amino acid sequence of SEQ ID NO: 62. The present invention also provides a kit containing this pair of two nucleic acids. In certain embodiments, the nucleic acid encoding the heavy chain variable region encodes the heavy chain of a felinized antibody and the corresponding nucleic acid encoding the light chain variable region encodes the light chain of that felinized antibody.

In still other specific embodiments, a nucleic acid of the present invention encodes a heavy chain variable region of a felinized antibody or antigen binding fragment thereof in which the heavy chain variable region comprises the amino acid sequence of SEQ ID NO: 59. In a related embodiment, a nucleic acid encodes the light chain variable region of the felinized antibody or antigen binding fragment thereof in which the light chain variable region comprises the amino acid sequence of SEQ ID NO: 61. The present invention further provides a pair of nucleic acids, wherein one of the pair of nucleic acids comprises a nucleotide sequence that encodes the heavy chain variable region that comprises the amino acid sequence of SEQ ID NO: 59 and the other of the pair of nucleic acids comprises a nucleotide sequence that encodes the light chain variable region that comprises the amino acid sequence of SEQ ID NO: 61. The present invention also provides a kit containing this pair of two nucleic acids. In certain embodiments, the nucleic acid encoding the heavy chain variable region encodes the heavy chain of a felinized antibody and the corresponding nucleic acid encoding the light chain variable region encodes the light chain of that felinized antibody.

In yet other specific embodiments, a nucleic acid of the present invention encodes a heavy chain variable region of a felinized antibody or antigen binding fragment thereof in which the heavy chain variable region comprises the amino acid sequence of SEQ ID NO: 59. In a related embodiment, a nucleic acid encodes the light chain variable region of the felinized antibody or antigen binding fragment thereof in which the light chain variable region comprises the amino acid sequence of SEQ ID NO: 62. The present invention further provides a pair of nucleic acids, wherein one of the pair of nucleic acids comprises a nucleotide sequence that encodes the heavy chain variable region that comprises the amino acid sequence of SEQ ID NO: 59 and the other of the pair of nucleic acids comprises a nucleotide sequence that encodes the light chain variable region that comprises the amino acid sequence of SEQ ID NO: 62. The present invention also provides a kit containing this pair of two nucleic acids. In certain embodiments, the nucleic acid encoding the heavy chain variable region encodes the heavy chain of a felinized antibody and the corresponding nucleic acid encoding the light chain variable region encodes the light chain of that felinized antibody.

In still other specific embodiments, a nucleic acid of the present invention encodes a heavy chain variable region of a felinized antibody or antigen binding fragment thereof in which the heavy chain variable region comprises the amino acid sequence of SEQ ID NO: 60. In a related embodiment, a nucleic acid encodes the light chain variable region of the felinized antibody or antigen binding fragment thereof in which the light chain variable region comprises the amino acid sequence of SEQ ID NO: 61. The present invention further provides a pair of nucleic acids, wherein one of the pair of nucleic acids comprises a nucleotide sequence that encodes the heavy chain variable region that comprises the amino acid sequence of SEQ ID NO: 60 and the other of the pair of nucleic acids comprises a nucleotide sequence that encodes the light chain variable region that comprises the amino acid sequence of SEQ ID NO: 61. The present invention also provides a kit containing this pair of two nucleic acids. In certain embodiments, the nucleic acid encoding the heavy chain variable region encodes the heavy chain of a felinized antibody and the corresponding nucleic acid encoding the light chain variable region encodes the light chain of that felinized antibody.

In yet other specific embodiments, a nucleic acid of the present invention encodes a heavy chain variable region of a felinized antibody or antigen binding fragment thereof in which the heavy chain variable region comprises the amino acid sequence of SEQ ID NO: 60. In a related embodiment, a nucleic acid encodes the light chain variable region of the felinized antibody or antigen binding fragment thereof in which the light chain variable region comprises the amino acid sequence of SEQ ID NO: 62. The present invention further provides a pair of nucleic acids, wherein one of the pair of nucleic acids comprises a nucleotide sequence that encodes the heavy chain variable region that comprises the amino acid sequence of SEQ ID NO: 60 and the other of the pair of nucleic acids comprises a nucleotide sequence that encodes the light chain variable region that comprises the amino acid sequence of SEQ ID NO: 62. The present invention also provides a kit containing this pair of two nucleic acids. In certain embodiments, the nucleic acid encoding the heavy chain variable region encodes the heavy chain of a felinized antibody and the corresponding nucleic acid encoding the light chain variable region encodes the light chain of that felinized antibody.

In still other specific embodiments, a nucleic acid of the present invention encodes a heavy chain of a felinized antibody or antigen binding fragment thereof in which the heavy chain comprises the amino acid sequence of SEQ ID NO: 64. In a related embodiment, a nucleic acid encodes the light chain of the felinized antibody or antigen binding fragment thereof in which the light chain comprises the amino acid sequence of SEQ ID NO: 68. The present invention further provides a pair of nucleic acids, wherein one of the pair of nucleic acids comprises a nucleotide sequence that encodes the heavy chain that comprises the amino acid sequence of SEQ ID NO: 64 and the other of the pair of nucleic acids comprises a nucleotide sequence that encodes the light chain that comprises the amino acid sequence of SEQ ID NO: 68. The present invention also provides a kit containing this pair of two nucleic acids.

In still other embodiments, a nucleic acid of the present invention encodes a heavy chain of a felinized antibody or antigen binding fragment thereof in which the heavy chain comprises the amino acid sequence of SEQ ID NO: 64. In a related embodiment, a nucleic acid encodes the light chain of the felinized antibody or antigen binding fragment thereof in which the light chain comprises the amino acid sequence of SEQ ID NO: 69. The present invention further provides a pair of nucleic acids, wherein one of the pair of nucleic acids comprises a nucleotide sequence that encodes the heavy chain that comprises the amino acid sequence of SEQ ID NO: 64 and the other of the pair of nucleic acids comprises a nucleotide sequence that encodes the light chain that comprises the amino acid sequence of SEQ ID NO: 69. The present invention also provides a kit containing this pair of two nucleic acids.

In specific embodiments, a nucleic acid of the present invention encodes a heavy chain of a felinized antibody or antigen binding fragment thereof in which the heavy chain comprises the amino acid sequence of SEQ ID NO: 65. In a related embodiment, a nucleic acid encodes the light chain of the felinized antibody or antigen binding fragment thereof in which the light chain comprises the amino acid sequence of SEQ ID NO: 68. The present invention further provides a pair of nucleic acids, wherein one of the pair of nucleic acids comprises a nucleotide sequence that encodes the heavy chain that comprises the amino acid sequence of SEQ ID NO: 65 and the other of the pair of nucleic acids comprises a nucleotide sequence that encodes the light chain that comprises the amino acid sequence of SEQ ID NO: 68. The present invention also provides a kit containing this pair of two nucleic acids.

In yet other specific embodiments, a nucleic acid of the present invention encodes a heavy chain of a felinized antibody or antigen binding fragment thereof in which the heavy chain comprises the amino acid sequence of SEQ ID NO: 65. In a related embodiment, a nucleic acid encodes the light chain of the felinized antibody or antigen binding fragment thereof in which the light chain comprises the amino acid sequence of SEQ ID NO: 69. The present invention further provides a pair of nucleic acids, wherein one of the pair of nucleic acids comprises a nucleotide sequence that encodes the heavy chain that comprises the amino acid sequence of SEQ ID NO: 65 and the other of the pair of nucleic acids comprises a nucleotide sequence that encodes the light chain that comprises the amino acid sequence of SEQ ID NO: 69. The present invention also provides a kit containing this pair of two nucleic acids.

In still other specific embodiments, a nucleic acid of the present invention encodes a heavy chain of a felinized antibody or antigen binding fragment thereof in which the heavy chain comprises the amino acid sequence of SEQ ID NO: 66. In a related embodiment, a nucleic acid encodes the light chain of the felinized antibody or antigen binding fragment thereof in which the light chain comprises the amino acid sequence of SEQ ID NO: 68. The present invention further provides a pair of nucleic acids, wherein one of the pair of nucleic acids comprises a nucleotide sequence that encodes the heavy chain that comprises the amino acid sequence of SEQ ID NO: 66 and the other of the pair of nucleic acids comprises a nucleotide sequence that encodes the light chain that comprises the amino acid sequence of SEQ ID NO: 68. The present invention also provides a kit containing this pair of two nucleic acids.

In yet other specific embodiments, a nucleic acid of the present invention encodes a heavy chain of a felinized antibody or antigen binding fragment thereof in which the heavy chain comprises the amino acid sequence of SEQ ID NO: 66. In a related embodiment, a nucleic acid encodes the light chain of the felinized antibody or antigen binding fragment thereof in which the light chain comprises the amino acid sequence of SEQ ID NO: 69. The present invention further provides a pair of nucleic acids, wherein one of the pair of nucleic acids comprises a nucleotide sequence that encodes the heavy chain that comprises the amino acid sequence of SEQ ID NO: 66 and the other of the pair of nucleic acids comprises a nucleotide sequence that encodes the light chain that comprises the amino acid sequence of SEQ ID NO: 69. The present invention also provides a kit containing this pair of two nucleic acids.

In still other specific embodiments, a nucleic acid of the present invention encodes a heavy chain of a felinized antibody or antigen binding fragment thereof in which the heavy chain comprises the amino acid sequence of SEQ ID NO: 67. In a related embodiment, a nucleic acid encodes the light chain of the felinized antibody or antigen binding fragment thereof in which the light chain comprises the amino acid sequence of SEQ ID NO: 68. The present invention further provides a pair of nucleic acids, wherein one of the pair of nucleic acids comprises a nucleotide sequence that encodes the heavy chain that comprises the amino acid sequence of SEQ ID NO: 67 and the other of the pair of nucleic acids comprises a nucleotide sequence that encodes the light chain that comprises the amino acid sequence of SEQ ID NO: 68. The present invention also provides a kit containing this pair of two nucleic acids.

In still other specific embodiments, a nucleic acid of the present invention encodes a heavy chain of a felinized antibody or antigen binding fragment thereof in which the heavy chain comprises the amino acid sequence of SEQ ID NO: 67. In a related embodiment, a nucleic acid encodes the light chain of the felinized antibody or antigen binding fragment thereof in which the light chain comprises the amino acid sequence of SEQ ID NO: 69. The present invention further provides a pair of nucleic acids, wherein one of the pair of nucleic acids comprises a nucleotide sequence that encodes the heavy chain that comprises the amino acid sequence of SEQ ID NO: 67 and the other of the pair of nucleic acids comprises a nucleotide sequence that encodes the light chain that comprises the amino acid sequence of SEQ ID NO: 69. The present invention also provides a kit containing this pair of two nucleic acids.

The present invention further provides expression vectors that comprise and express one or more of the nucleic acids of the present invention. In particular embodiments, the expression vector comprises and expresses a nucleic acid encoding a heavy chain of a felinized antibody of the present invention and a nucleic acid encoding a light chain of that felinized antibody. The present invention also provides host cells that comprise one or more expression vectors of the present invention.

The present invention also provides pharmaceutical compositions comprising the felinized antibodies and/or antigen binding fragments of the antibodies and a pharmaceutically acceptable carrier or diluent. In addition, pharmaceutical compositions are provided that comprise a nucleic acid encoding a heavy chain of a felinized antibody of the present invention and a nucleic acid encoding a light chain of that felinized antibody and a pharmaceutically acceptable carrier or diluent. In other embodiments, the pharmaceutical compositions comprise a nucleic acid encoding both a heavy chain of a felinized antibody of the present invention and a light chain of that felinized antibody. In yet other embodiments, the pharmaceutical compositions comprise a pharmaceutically acceptable carrier or diluent and an expression vector that comprises one or more nucleic acids encoding a heavy chain of a felinized antibody of the present invention and a light chain of that felinized antibody and thereby, can express the felinized antibody and/or antigen binding fragments of the antibody of the present invention, in vivo.

The present invention further provides methods of treating a condition associated with pain in an animal subject. The method of treatment can comprise administering to an animal subject in need thereof, a therapeutically effective amount of a pharmaceutical composition of the present invention. In certain embodiments, the method is used for the treatment of osteoarthritis. In other embodiments, the method is used for the treatment of hyperalgesia. In still other embodiments, the method is used for the treatment of allodynia. In yet other embodiments, the method is used for the treatment of pain. In still other embodiments, the method is used for the treatment of any combination of osteoarthritis, hyperalgesia, allodynia, and/or pain. When a felinized antibody or antigen binding fragment thereof is administered, the animal subject is preferably a feline.

The present invention also provides methods of producing a felinized antibody or antigen binding fragment thereof that binds feline NGF. In particular embodiments, the method includes culturing one or more host cells comprising one or more expression vectors that encode and express the light chain of a felinized antibody of the present invention and/or the heavy chain of that felinized antibody in a culture medium under conditions in which the nucleic acid is expressed, thereby producing a polypeptide comprising the light chain of a felinized antibody of the present invention, and/or the heavy chain of that felinized antibody. The polypeptides are then recovered from the one or more host cells or culture medium. In certain embodiments, the polypeptides comprising the light chain of a felinized antibody of the present invention and the polypeptides comprising the heavy chain of that felinized antibody are combined with each under conditions that are conducive to form a felinized antibody.

The present invention further provides a pair of host cells, where in one of the pair of host cells comprises an expression vector that comprises one of a pair of nucleic acids that comprises a nucleotide sequence that encodes the heavy chain of a specific felinized antibody of present invention, whereas the other of the pair of host cells comprises an expression vector that comprises the other of the pair of nucleic acids that comprises the nucleotide sequence that encodes the light chain of said specific felinized antibody. The present invention further provides a method of producing a felinized antibody of the present invention that binds feline NGF comprising culturing each one of the pair of host cells in a culture medium either individually or in combination under conditions wherein the nucleic acids are expressed, thereby producing a polypeptide comprising the light chain of the felinized antibody, the heavy chain of the felinized antibody, or both and then recovering the light chain of the felinized antibody, the heavy chain of the felinized antibody, or both from the pair of host cells or culture medium.

These and other aspects of the present invention will be better appreciated by reference to the following Brief Description of the Drawings and the Detailed Description.

### BRIEF DESCRIPTION OF THE DRAWINGS

FIG. 1 depicts the binding of human-canine chimeric Fulranumab (Ful Chimeric) and variants of caninized Fulranumab (cFul) antibodies to canine NGF.

Ful Chimeric (•), cFulVH1L1 (▪), cFulVH1L2 (▴), cFulVH2L1 (▾), cFulVH2L2 (♦), and mab control (∘).

FIG. 2 depicts the binding of human-canine chimeric Fasinumab (Fas Chimeric) and individual caninized Fasinumab (cFas) antibodies to canine NGF. Fas Chimeric (•), cFasVH2L2 (▪), cFasVH2L3 (▴), and mab control (∘).

FIG. 3 shows the binding of canine NGF to the canine TrkA receptor. The binding of canine NGF to canine NGF receptor (TrkA) was determined by ELISA. Canine NGF (•).

FIG. 4 depicts the inhibition of canine NGF binding to canine TrkA receptor by the human-canine chimeric Fulranumab or by individual caninized antibodies. Ful Chimeric (•), cFulVH1L1 (▪), cFulVH1L2 (▴), cFulVH2L1 (▾), cFulVH2L2 (♦), and mab control (∘). FIG. 5 shows the stimulation of TF-1 cell proliferation by canine NGF. [Canine NGF (•)].

FIG. 6 shows the inhibition of TF-1 cell proliferation by human-canine chimeric Fulranumab (Ful Chimeric) or individual caninized anti-NGF antibodies. Ful Chimeric (•), cFulVH1L1 (▪), cFulVH1L2 (▴), cFulVH2L1 (▾), cFulVH2L2 (♦), and mab control (∘).

FIG. 7 depicts the binding of human-feline chimeric Fulranumab (Ful Chimeric) and variants of felinized antibodies (fFul) to feline NGF. Binding was determined with an ELISA assay. The Ful Chimeric (•), fFulH2L1 (▪), fFulH2L2 (▴), fFulH4L1 (▾), and fFulVH4L2 (♦).

FIG. 8 depicts the dose-dependent ability of the C-terminal portion of feline NGF (•) to stimulate proliferation of TF-1 cells.

FIG. 9 depicts the ability of human-feline chimeric Fulranumab (Ful H-F), the listed felinized variants of Fulranumab and isotype control mAb (mAb ctrl) to block TF-1 cell proliferation. Human-feline chimeric Fulranumab (Ful H-F) (•), fFulH2L1 (▪), fFulH2L2 (▴), fFulH4L1 (▾), fFulH4L2 (♦), and mab control (∘).

### DETAILED DESCRIPTION OF THE INVENTION

In response to need for better therapies for pain in canines and felines, the present invention provides formulations and methodology that can achieve a significant effect to relieve the pain associated with and/or due to NGF. Accordingly, it was surprisingly found that whereas caninized and felinized antibodies comprising a set of CDRs from one antibody first raised against human NGF could bind tightly to canine and feline NGF, respectively, a caninized antibody comprising a set of CDRs from another antibody first raised against human NGF could not measurably bind to canine NGF. This was true even though both corresponding human-canine chimeric constructs could tightly bind to canine NGF.

### Abbreviations

Throughout the detailed description and examples of the invention the following abbreviations will be used:
ADCC Antibody-dependent cellular cytotoxicity
CDC Complement-dependent cyotoxicity
CDR Complementarity determining region in the immunoglobulin variable regions, defined using the Kabat numbering system
EC50 concentration resulting in 50% efficacy or binding
ELISA Enzyme-linked immunosorbant assay
FR Antibody framework region: the immunoglobulin variable regions excluding the CDR regions.
IC50 concentration resulting in 50% inhibition
IgG Immunoglobulin G
Kabat An immunoglobulin alignment and numbering system pioneered by Elvin A. Kabat [Sequences of Proteins of Immunological Interest, 5th Ed. Public Health Service, National Institutes of Health, Bethesda, Md. (1991)]
mAb Monoclonal antibody (also Mab or MAb)
V region The segment of IgG chains which is variable in sequence between different antibodies.
VH Immunoglobulin heavy chain variable region
VL Immunoglobulin light chain variable region

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Activity” may refer to modulation of components of the innate or the adaptive immune systems.

“Administration” and “treatment”, as it applies to an animal, e.g., a canine or feline subject, cell, tissue, organ, or biological fluid, refers to contact of an exogenous pharmaceutical, therapeutic, diagnostic agent, or composition to the animal e.g., a canine or feline subject, cell, tissue, organ, or biological fluid. Treatment of a cell encompasses contact of a reagent to the cell, as well as contact of a reagent to a fluid, where the fluid is in contact with the cell.

“Administration” and “treatment” also mean in vitro and ex vivo treatments, e.g., of a cell, by a reagent, diagnostic, binding compound, or by another cell. The term “subject” includes any organism, preferably a non-human animal, more preferably a mammal (e.g., canine or feline) and most preferably a canine or a feline.

“Treat” or “treating” means to administer a therapeutic agent, such as a composition containing any of the antibodies of the present invention, internally or externally to e.g., a canine or feline subject or patient having one or more symptoms, or being suspected of having a condition, for which the agent has therapeutic activity. Typically, the agent is administered in an amount effective to alleviate and/or ameliorate one or more disease/condition symptoms in the treated subject or population, whether by inducing the regression of or inhibiting the progression of such symptom(s) by any clinically measurable degree. The amount of a therapeutic agent that is effective to alleviate any particular disease/condition symptom (also referred to as the “therapeutically effective amount”) may vary according to factors such as the disease/condition state, age, and weight of the canine or feline, and the ability of the pharmaceutical composition to elicit a desired response in the subject. Whether a disease/condition symptom has been alleviated or ameliorated can be assessed by any clinical measurement typically used by veterinarians or other skilled healthcare providers to assess the severity or progression status of that symptom. While an embodiment of the present invention (e.g., a treatment method or article of manufacture) may not be effective in alleviating the target disease/condition symptom(s) in every subject, it should alleviate the target disease/condition symptom(s) in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Treatment,” as it applies to a veterinary (e.g., canine or feline) or research subject, refers to therapeutic treatment, as well as research and diagnostic applications. “Treatment” as it applies to a veterinary (e.g., canine or feline), or research subject, or cell, tissue, or organ, encompasses contact of the antibodies of the present invention to e.g., a canine or feline), a cell, tissue, physiological compartment, or physiological fluid.

As used herein, the term “canine” includes all domestic dogs, Canis lupus familiaris or Canis familiaris, unless otherwise indicated.

As used herein, the term “feline” refers to any member of the Felidae family. Members of this family include wild, zoo, and domestic members, including domestic cats, pure-bred and/or mongrel companion cats, show cats, laboratory cats, cloned cats, and wild or feral cats.

As used herein the term “canine frame” refers to the amino acid sequence of the heavy chain and light chain of a canine antibody other than the hypervariable region residues defined herein as CDR residues. With regard to a caninized antibody, in the majority of embodiments the amino acid sequences of the native canine CDRs are replaced with the corresponding foreign CDRs (e.g., those from a mouse or human) in both chains. Optionally the heavy and/or light chains of the canine antibody may contain some foreign non-CDR residues, e.g., so as to preserve the conformation of the foreign CDRs within the caninized antibody, and/or to modify the Fc region function, as exemplified below and/or disclosed in U.S. Pat. No. 10,106,607 B2, hereby incorporated by reference herein in its entirety.

As used herein the term “feline frame” refers to the amino acid sequence of the heavy chain and light chain of a feline antibody other than the hypervariable region residues defined herein as CDR residues. With regard to a felinized antibody, in the majority of embodiments the amino acid sequences of the native feline CDRs are replaced with the corresponding foreign CDRs (e.g., those from a mouse or human) in both chains. Optionally the heavy and/or light chains of the feline antibody may contain some foreign non-CDR residues, e.g., so as to preserve the conformation of the foreign CDRs within the felinized antibody, and/or to modify the Fc region function, as exemplified below and/or disclosed for caninized antibodies in U.S. Pat. No. 10,106,607 B2, hereby incorporated by reference herein in its entirety.

The “Fragment crystallizable region” abbreviated as “Fc” or used interchangeably with “Fc region” corresponds to the CH3-CH2 portion of an antibody that interacts with cell surface receptors called Fc receptors. The canine fragment crystallizable region (cFc region) of each of the four canine IgGs were first described by Tang et al. [Vet. Immunol. Immunopathol. 80: 259-270 (2001); see also, Bergeron et al., Vet. Immunol. Immunopathol. 157: 31-41 (2014) and U.S. Pat. No. 10,106,607 B2].

As used herein the canine Fc (cFc) “IgG-Bm” is canine IgG-B Fc comprising two (2) amino acid residue substitutions, D31A and N63A, as in the amino acid sequence of SEQ ID NO: 20 of IgG-B (see below) and preferably without the c-terminal lysine (“K”) i.e., SEQ ID NO: 51). Both the aspartic acid residue (D) at position 31 of SEQ ID NO: 50 and the asparagine residue (N) at position 63 of SEQ ID NO: 50, are substituted by an alanine residue (A) in IgG-Bm. These two amino acid residue substitutions serve to significantly diminish the antibody-dependent cytotoxicity (ADCC) and complement-dependent cytotoxicity (CDC) of the naturally occurring canine IgG-B [see, U.S. Pat. No. 10,106,607 B2, the contents of which are hereby incorporated by reference in their entirety]. Further amino acid substitutions to the IgG-Bm are also envisioned, which parallel those which can be made in IgG-B. Analogous changes in the amino acid sequences of feline heavy constant regions, IgG1a, and IgG1b also are provided in the Examples below; such modifications are denoted as “IgG1am” or “IgG1bm” respectively.

As used herein, a “substitution of an amino acid residue” with another amino acid residue in an amino acid sequence of an antibody for example, is equivalent to “replacing an amino acid residue” with another amino acid residue and denotes that a particular amino acid residue at a specific position in the amino acid sequence has been replaced by (or substituted for) by a different amino acid residue. Such substitutions can be particularly designed i.e., purposefully replacing an alanine with a serine at a specific position in the amino acid sequence by e.g., recombinant DNA technology. Alternatively, a particular amino acid residue or string of amino acid residues of an antibody can be replaced by one or more amino acid residues through more natural selection processes e.g., based on the ability of the antibody produced by a cell to bind to a given region on that antigen, e.g., one containing an epitope or a portion thereof, and/or for the antibody to comprise a particular CDR that retains the same canonical structure as the CDR it is replacing. Such substitutions/replacements can lead to “variant” CDRs and/or variant antibodies.

As used herein, the term “antibody” refers to any form of antibody that exhibits the desired biological activity. An antibody can be a monomer, dimer, or larger multimer. Thus, it is used in the broadest sense and specifically covers, but is not limited to, monoclonal antibodies (including full length monoclonal antibodies), polyclonal antibodies, multi-specific antibodies (e.g., bispecific antibodies), caninized antibodies, fully canine antibodies, chimeric antibodies and camelized single domain antibodies. “Parental antibodies” are antibodies obtained by exposure of an immune system to an antigen prior to modification of the antibodies for an intended use, such as caninization or felinization of an antibody for use as a canine therapeutic antibody.

As used herein, an antibody of the present invention that “blocks” or is “blocking” or is “blocking the binding” of e.g., a canine ligand to its binding partner (e.g., its receptor) or a feline ligand to its binding partner (e.g., its receptor), is an antibody that blocks (partially or fully) the binding of the ligand and its receptor and vice versa, as determined in standard binding assays (e.g., BIACore®, ELISA, or flow cytometry).

Typically, an antibody or antigen binding fragment of the invention retains at least 10% of its canine or feline antigen binding activity (when compared to the parental antibody) when that activity is expressed on a molar basis. Preferably, an antibody or antigen binding fragment of the invention retains at least 20%, 50%, 70%, 80%, 90%, 95% or 100% or more of the canine antigen binding affinity as the parental antibody. It is also intended that an antibody or antigen binding fragment of the invention can include conservative or non-conservative amino acid substitutions (referred to as “conservative variants” or “function conserved variants” of the antibody) that do not substantially alter its biologic activity.

“Isolated antibody” refers to the purification status and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

As used herein, an antibody is said to bind specifically to a polypeptide comprising a given antigen sequence (in this case a portion of the amino acid sequence of canine and/or feline NGF) if it binds to polypeptides comprising the portion of the amino acid sequence of canine and/or feline NGF, but does not bind to other canine (or feline) proteins lacking that portion of the sequence of canine and/or feline NGF. For example, an antibody that specifically binds to a polypeptide comprising canine and/or feline NGF, may bind to a FLAG*-tagged form of canine and/or feline NGF, but will not bind to other FLAG*-tagged canine and/or feline proteins.

As used herein, unless otherwise indicated, “antibody fragment” or “antigen binding fragment” refers to antigen binding fragments of antibodies, i.e. antibody fragments that retain the ability to bind specifically to the antigen (e.g., canine and/or feline NGF) bound by the full-length antibody, e.g. fragments that retain one or more CDR regions. Examples of antigen binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

An antibody, or binding compound derived from the antigen-binding site of an antibody, binds to its canine and/or feline antigen, or a variant or mutein thereof, “with specificity” when it has an affinity for that canine and/or feline antigen or a variant or mutein thereof which is at least ten-times greater, more preferably at least 20-times greater, and even more preferably at least 100-times greater than its affinity for any other canine and/or feline antigen tested. An antibody that binds canine and/or feline NGF “with specificity” may still bind an NGF from another species (e.g., human NGF).

As used herein, a “chimeric antibody” is an antibody having the variable domain from a first antibody and the constant domain from a second antibody, where the first and second antibodies are from different species. [U.S. Pat. No. 4,816,567; and Morrison et al., Proc. Natl. Acad. Sci. USA 81: 6851-6855 (1984)]. Typically the variable domains are obtained from an antibody from an experimental animal (the “parental antibody”), such as a rodent (or a rodent that comprises a human immune system) and the constant domain sequences are obtained from the animal subject antibodies, e.g., canine or feline so that the resulting chimeric antibody will be less likely to elicit an adverse immune response in a canine or feline subject respectively, than the parental (e.g., rodent) antibody.

As used herein, the term “caninized antibody” refers to forms of antibodies that contain sequences from both canine and non-canine (e.g., mouse or human) antibodies, whereas, as used herein, the term “felinized antibody” refers to forms of antibodies that contain sequences from both feline and non-feline (e.g., mouse or human) antibodies. In general, the caninized antibody or felinized antibody will comprise substantially all of at least one or more typically, two variable domains in which all or substantially all of the hypervariable loops correspond to those of a non-canine or a non-feline immunoglobulin respectively (e.g., comprising 6 CDRs as exemplified below), and all or substantially all of the framework (FR) regions (and typically all or substantially all of the remaining frame) are those of a canine immunoglobulin or a feline immunoglobulin sequence, respectively.

A caninized antibody can comprise both the three heavy chain CDRs and the three light chain CDRS from e.g., a human anti-human NGF antibody together with a canine frame or a modified canine frame. A modified canine frame comprises one or more amino acids changes as exemplified herein that further optimize the effectiveness of the caninized antibody, e.g., to increase its binding to its canine antigen and/or its ability to block the binding of that canine antigen to the canine antigen's natural binding partner.

Similarly, a felinized antibody can comprise both the three heavy chain CDRs and the three light chain CDRS from e.g., a human anti-human NGF antibody together with a feline frame or a modified feline frame. A modified feline frame comprises one or more amino acids changes as exemplified herein that further optimize the effectiveness of the felinized antibody, e.g., to increase its binding to its feline antigen and/or its ability to block the binding of that feline antigen to the feline antigen's natural binding partner.

The variable regions of each light/heavy chain pair form the antibody binding site. Thus, in general, an intact antibody has two binding sites. Except in bifunctional or bispecific antibodies, the two binding sites are, in general, the same. Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat, Adv. Prot. Chem. 32:1-75 (1978); Kabat, et al., J. Biol. Chem. 252:6609-6616 (1977); Chothia, et al., J. Mol. Biol. 196:901-917 (1987) or Chothia, et al., Nature 342:878-883 (1989)].

As used herein, the term “hypervariable region” refers to the amino acid residues of an antibody that are responsible for antigen-binding. The hypervariable region comprises amino acid residues from a “complementarity determining region” or “CDR” (i.e., LCDR1 or CDRL1, LCDR2 or CDRL2, and LCDR3 or CDRL3 in the light chain variable domain and HCDR1 or CDRH1, HCDR2 or CDRH2, and HCDR3 or CDRH3, in the heavy chain variable domain). [See Kabat et al. Sequences of Proteins of Immunological Interest, 5th Ed. Public Health Service, National Institutes of Health, Bethesda, Md. (1991), defining the CDR regions of an antibody by sequence; see also Chothia and Lesk, J. Mol. Biol. 196: 901-917 (1987) defining the CDR regions of an antibody by structure]. As used herein, the term “framework” or “FR” residues refers to those variable domain residues other than the hypervariable region residues defined herein as CDR residues.

In canine, there are four IgG heavy chains referred to as A, B, C, and D. These heavy chains represent four different subclasses of dog IgG, which are referred to as IgG-A (or IgGA), IgG-B (or IgGB), IgG-C (or IgGC) and IgG-D (or IgGD). Each of the two heavy chains consists of one variable domain (VH) and three constant domains referred to as CH-1, CH-2, and CH-3. The CH-1 domain is connected to the CH-2 domain via an amino acid sequence referred to as the “hinge” or alternatively as the “hinge region”. The two known light canine chain subtypes are referred to as lambda and kappa.

“Homology”, as used herein, refers to sequence similarity between two polynucleotide sequences or between two polypeptide sequences when they are optimally aligned. When a position in both of the two compared sequences is occupied by the same base or amino acid residue, e.g., if a position in each of two DNA molecules is occupied by adenine, then the molecules are homologous at that position. The percent of homology is the number of homologous positions shared by the two sequences divided by the total number of positions compared ×100. For example, if 6 of 10 of the positions in two sequences are matched or homologous when the sequences are optimally aligned then the two sequences are 60% homologous. Generally, the comparison is made when two sequences are aligned to give maximum percent homology. Sequence identity refers to the degree to which the amino acids of two polypeptides are the same at equivalent positions when the two sequences are optimally aligned. As used herein one amino acid sequence is 100% “identical” to a second amino acid sequence when the amino acid residues of both sequences are identical.

Accordingly, an amino acid sequence is 50% “identical” to a second amino acid sequence when 50% of the amino acid residues of the two amino acid sequences are identical. The sequence comparison is performed over a contiguous block of amino acid residues comprised by a given protein, e.g., a protein, or a portion of the polypeptide being compared. In particular embodiments, selected deletions or insertions that could otherwise alter the correspondence between the two amino acid sequences are taken into account. Sequence similarity includes identical residues and nonidentical, biochemically related amino acids, e.g., biochemically related amino acids that share similar properties and may be interchangeable.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity [see, e.g., Watson et al., Molecular Biology of the Gene, The Benjamin/Cummings Pub. Co., p. 224 (4th Ed.; 1987)]. In addition, substitutions of structurally or functionally similar amino acids are less likely to disrupt biological activity. Exemplary conservative substitutions are set forth in Table A directly below.

TABLE A
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala; Gly |
| Ser (S) | Thr |
| Thr (T) | Ser |
| Trp (W) | Tyr; Phe |
| Tyr (Y) | Trp; Phe |
| Val (V) | Ile; Leu |

Function-conservative variants of the antibodies of the invention are also contemplated by the present invention. “Function-conservative variants,” as used herein, refers to antibodies or fragments in which one or more amino acid residues have been changed without altering a desired property, such an antigen affinity and/or specificity. Such variants include, but are not limited to, replacement of an amino acid with one having similar properties, such as the conservative amino acid substitutions of Table A above.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The present invention provides isolated caninized and felinized antibodies of the present invention, methods of use of the antibodies in the treatment of a condition e.g., the treatment of osteoarthritis in canines or felines.

The nucleic acid and amino acid sequences of these four heavy chains were first identified by Tang et al. [Vet. Immunol. Immunopathol. 80: 259-270 (2001)]. The amino acid and nucleic sequences for these heavy chains are also available from the GenBank data bases.

For example, the amino acid sequence of IgGA heavy chain has accession number AAL35301.1, IgGB has accession number AAL35302.1, IgGC has accession number AAL35303.1, and IgGD has accession number (AAL35304.1). Canine antibodies also contain two types of light chains, kappa and lambda. The DNA and amino acid sequence of these light chains can be obtained from GenBank Databases. For example, the kappa light chain amino acid sequence has accession number ABY 57289.1 and the lambda light chain has accession number ABY 55569.1.

The known amino acid sequences of the four unmodified canine Fcs are:

cIgG-A
[SEQ ID NO: 49]
LGGPSVLIFPPKPKDILRITRTPEVTCVVLDLGREDPEVQISWFVDGKEV
HTAKTQSREQQFNGTYRVVSVLPIEHQDWLTGKEFKCRVNHIDLPSPIER
TISKARGRAHKPSVYVLPPSPKELSSSDTVSITCLIKDFYPPDIDVEWQS
NGQQEPERKHRMTPPQLDEDGSYFLYSKLSVDKSRWQQGDPFTCAVMHET
LQNHYTDLSLSHSPGK
cIgG-B
[SEQ ID NO: 50]
LGGPSVFIFPPKPKDTLLIARTPEVTCVVVDLDPEDPEVQISWFVDGKQM
QTAKTQPREEQFNGTYRVVSVLPIGHQDWLKGKQFTCKVNNKALPSPIER
TISKARGQAHQPSVYVLPPSREELSKNTVSLTCLIKDFFPPDIDVEWQSN
GQQEPESKYRTTPPQLDEDGSYFLYSKLSVDKSRWQRGDTFICAVMHEAL
HNHYTQESLSHSPGK
cIgG-C
[SEQ ID NO: 52]
LGGPSVFIFPPKPKDILVTARTPTVTCVVVDLDPENPEVQISWFVDSKQV
QTANTQPREEQSNGTYRVVSVLPIGHQDWLSGKQFKCKVNNKALPSPIEE
IISKTPGQAHQPNVYVLPPSRDEMSKNTVTLTCLVKDFFPPEIDVEWQSN
GQQEPESKYRMTPPQLDEDGSYFLYSKLSVDKSRWQRGDTFICAVMHEAL
HNHYTQISLSHSPGK
cIgG-D
[SEQ ID NO: 53]
LGGPSVFIFPPKPKDILRITRTPEITCVVLDLGREDPEVQISWFVDGKEV
HTAKTQPREQQFNSTYRVVSVLPIEHQDWLTGKEFKCRVNHIGLPSPIER
TISKARGQAHQPSVYVLPPSPKELSSSDTVTLTCLIKDFFPPEIDVEWQS
NGQPEPESKYHTTAPQLDEDGSYFLYSKLSVDKSRWQQGDTFTCAVMHEA
LQNHYTDLSLSHSPGK

In the present invention, the amino acid sequence for each of the four canine IgG Fc regions is based on the identified boundary of CH1 and CH2 domains as determined by Tang et al, supra.

The present invention also provides antibodies of the present invention that comprise a canine fragment crystallizable region (cFc region) in which the cFc region has been genetically modified to augment, decrease, or eliminate one or more effector functions. In one aspect of the present invention, the genetically modified cFc region decreases or eliminates one or more effector functions. In another aspect of the invention the genetically modified cFc region augments one or more effector function. In certain embodiments, the genetically modified cFc region is a genetically modified canine IgGB Fc region. In another such embodiment, the genetically modified cFc region is a genetically modified canine IgGC Fc region. In a particular embodiment, the effector function is antibody-dependent cytotoxicity (ADCC) that is augmented, decreased, or eliminated. In another embodiment, the effector function is complement-dependent cytotoxicity (CDC) that is augmented, decreased, or eliminated. In yet another embodiment, the cFc region has been genetically modified to augment, decrease, or eliminate both the ADCC and the CDC.

In specific embodiments of the invention, besides binding canine NGF, a canine or caninized antibody against its antigen of the present invention optimally has two attributes:
1. Lack of effector functions such as antibody-dependent cytotoxicity (ADCC) and complement-dependent cytotoxicity (CDC), and
2. be readily purified on a large scale using industry standard technologies such as that based on protein A chromatography.

None of the naturally occurring canine IgG isotypes satisfy both criteria. For example, IgG-B can be purified using protein A, but has high level of ADCC activity. On the other hand, IgG-A binds weakly to protein A, but also displays ADCC activity. Moreover, neither IgG-C nor IgG-D can be purified on protein A columns, although IgG-D displays no ADCC activity. (IgG-C has considerable ADCC activity). One way the present invention addresses these issues in certain embodiments is by providing modified canine IgG-B antibodies of the present invention specific to an antigen of the present invention that lack the effector functions such as ADCC and can be easily purified using industry standard protein A chromatography. As disclosed in the Examples below, two heavy chain constant regions of feline IgGs: IgG1a, IgG1b, can be modified analogously.

The amino acid sequence of IgG-B, SEQ ID NO: 50 is:

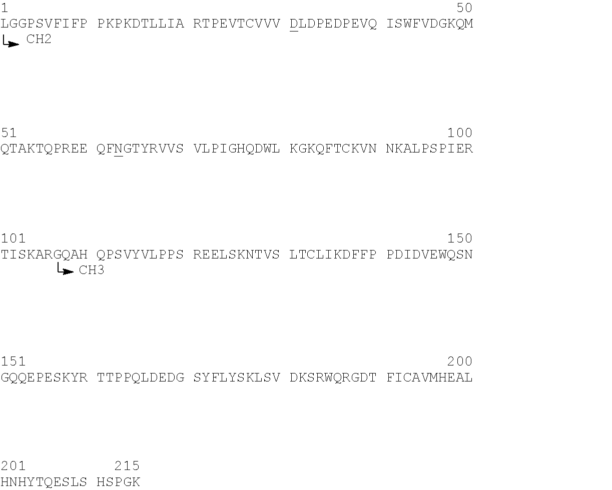

The amino acid sequence of IgG-Bm, SEQ ID NO: 51, is provided below.

LGGPSVFIFPPKPKDTLLIARTPEVTCVVVALDPEDPEVQISWFVDGKQM
QTAKTQPREEQFAGTYRVVSVLPIGHQDWLKGKQFTCKVNNKALPSPIER
TISKARGQAHQPSVYVLPPSREELSKNTVSLTCLIKDFFPPDIDVEWQSN
GQQEPESKYRTTPPQLDEDGSYFLYSKLSVDKSRWQRGDTFICAVMHEAL
HNHYTQESLSHSPG

The amino acid sequence of IgG-Bm, SEQ ID NO: 20, with the C-Terminal lysine (K):

LGGPSVFIFPPKPKDTLLIARTPEVTCVVVALDPEDPEVQISWFVDGKQM
QTAKTQPREEQFAGTYRVVSVLPIGHQDWLKGKQFTCKVNNKALPSPIER
TISKARGQAHQPSVYVLPPSREELSKNTVSLTCLIKDFFPPDIDVEWQSN
GQQEPESKYRTTPPQLDEDGSYFLYSKLSVDKSRWQRGDTFICAVMHEAL
HNHYTQESLSHSPGK

In order to generate variants of canine IgG that lack effector functions, a number of mutant canine IgGB heavy chains were generated. These variants may include one or more of the following single or combined substitutions in the Fc portion of the heavy chain amino acid sequence: P4A, D31A, N63A, G64P, T65A, A93G, and P95A. Variant heavy chains (i.e., containing such amino acid substitutions) are cloned into expression plasmids and are transfected into HEK 293 cells along with a plasmid containing the gene encoding a light chain. Intact antibodies are expressed and purified from HEK 293 cells and then can be evaluated for binding to Fc_{γ}RI and C1q to assess their potential for mediation of immune effector functions. [See, U.S. Pat. No. 10,106,607 B2, the contents of which are hereby incorporated by reference in its entirety.]

The present invention also provides modified canine IgG-Ds which in place of its natural IgG-D hinge region they comprise a hinge region from:

| | IgG-A: |
|---|---|
| SEQ ID NO: 45 | |
| | FNECRCTDTPPCPVPEP |
| | IgG-B: |
| SEQ ID NO: 46 | |
| | PKRENGRVPRPPDCPKCPAPEM; |
| | or |
| | IgG-C: |
| SEQ ID NO: 47 | |
| | AKECECKCNCNNCPCPGCGL. |

Alternatively, the IgG-D hinge region can be genetically modified by replacing a serine residue with a proline residue, i.e., PKESTCKCIPPCPVPES, SEQ ID NO: 48 (with the proline residue (P) underlined and in bold substituting for the naturally occurring serine residue). Such modifications can lead to a canine IgG-D lacking fab arm exchange. The modified canine IgG-Ds can be constructed using standard methods of recombinant DNA technology [e.g., Maniatis et al., Molecular Cloning, A Laboratory Manual (1982)]. In order to construct these variants, the nucleic acids encoding the amino acid sequence of canine IgG-D can be modified so that it encodes the modified IgG-Ds. The modified nucleic acid sequences are then cloned into expression plasmids for protein expression.

The six complementary determining regions (CDRs) of a caninized mouse or human anti-NGF antibody, as described herein can comprises a canine antibody kappa (k) or lambda (l) light chain comprising a mouse or human light chain LCDR1, LCDR2, and LCDR3 and a canine antibody heavy chain comprising a mouse or human heavy chain HCDR1, HCDR2, and HCDR3.

Analogously, the present invention further provides felinized mouse or human antibodies of the present invention, including isolated felinized mouse or human anti-human NGF antibodies, that bind to Feline NGF and that preferably also block the binding of that feline NGF to feline TrkA. The present invention also provides felinized NGF antibodies and methods of use of the felinized antibodies of the present invention in the treatment of pain e.g., osteoarthritis in felines. In addition, the present invention provides full length felinized heavy chains that can be matched with corresponding light chains to make a felinized antibody. Accordingly, the present invention also provides felinized mouse or human anti-NGF antibodies (including isolated felinized human anti-human NGF antibodies) of the present invention and methods of use of the antibodies of the present invention in the treatment of a condition e.g., the treatment of pain in felines.

### Nucleic Acids

The present invention also comprises the nucleic acids encoding the antibodies of the present invention (see also e.g., Examples below).

Also included in the present invention are nucleic acids that encode immunoglobulin polypeptides comprising amino acid sequences that are at least about 70% identical, preferably at least about 80% identical, more preferably at least about 90% identical and most preferably at least about 95% identical (e.g., 95%, 96%, 97%, 98%, 99%, 100%) to the amino acid sequences of the caninized and felinized antibodies, with the exception of the CDRs which do not change, provided herein when the comparison is performed by a BLAST algorithm wherein the parameters of the algorithm are selected to give the largest match between the respective sequences over the entire length of the respective reference sequences.

The present invention further provides nucleic acids that encode immunoglobulin polypeptides comprising amino acid sequences that are at least about 70% similar, preferably at least about 80% similar, more preferably at least about 90% similar and most preferably at least about 95% similar (e.g., 95%, 96%, 97%, 98%, 99%, 100%) to any of the reference amino acid sequences when the comparison is performed with a BLAST algorithm, wherein the parameters of the algorithm are selected to give the largest match between the respective sequences over the entire length of the respective reference sequences, are also included in the present invention.

As used herein, nucleotide and amino acid sequence percent identity can be determined using C, MacVector (MacVector, Inc. Cary, NC 27519), Vector NTI (Informax, Inc. MD), Oxford Molecular Group PLC (1996) and the Clustal W algorithm with the alignment default parameters, and default parameters for identity. These commercially available programs can also be used to determine sequence similarity using the same or analogous default parameters. Alternatively, an Advanced Blast search under the default filter conditions can be used, e.g., using the GCG (Genetics Computer Group, Program Manual for the GCG Package, Version 7, Madison, Wisconsin) pileup program using the default parameters.

The following references relate to BLAST algorithms often used for sequence analysis: BLAST ALGORITHMS: Altschul, S. F., et al., J. Mol. Biol. 215:403-410 (1990); Gish, W., et al., Nature Genet. 3:266-272 (1993); Madden, T. L., et al., Meth. Enzymol. 266:131-141(1996); Altschul, S. F., et al., Nucleic Acids Res. 25:3389-3402 (1997); Zhang, J., et al., Genome Res. 7:649-656 (1997); Wootton, J. C., et al., Comput. Chem. 17:149-163 (1993); Hancock, J. M. et al., Comput. Appl. Biosci. 10:67-70 (1994); ALIGNMENT SCORING SYSTEMS: Dayhoff, M. O., et al., “A model of evolutionary change in proteins.” in Atlas of Protein Sequence and Structure, vol. 5, suppl. 3. M. O. Dayhoff (ed.), pp. 345-352, (1978); Natl. Biomed. Res. Found., Washington, DC; Schwartz, R. M., et al., “Matrices for detecting distant relationships.” in Atlas of Protein Sequence and Structure, vol. 5, suppl. 3.” (1978), M. O. Dayhoff (ed.), pp. 353-358 (1978), Natl. Biomed. Res. Found., Washington, DC; Altschul, S. F., J. Mol. Biol. 219:555-565 (1991); States, D. J., et al., Methods 3:66-70(1991); Henikoff, S., et al., Proc. Natl. Acad. Sci. USA 89:10915-10919 (1992); Altschul, S. F., et al., J. Mol. Evol. 36:290-300 (1993); ALIGNMENT STATISTICS: Karlin, S., et al., Proc. Natl. Acad. Sci. USA 87:2264-2268 (1990); Karlin, S., et al., Proc. Natl. Acad. Sci. USA 90:5873-5877 (1993); Dembo, A., et al., Ann. Prob. 22:2022-2039 (1994); and Altschul, S. F. “Evaluating the statistical significance of multiple distinct local alignments.” in Theoretical and Computational Methods in Genome Research (S. Suhai, ed.), pp. 1-14, Plenum, New York (1997).

### Antibody Protein Engineering

By way of example, and not limitation, as indicated above, the canine heavy chain constant region can be from IgG-A, IgG-B, IgG-C, IgG-D, and the corresponding cFc can be a modified cFc, such as the IgG-Bm of the IgG-B heavy constant region used herein [see, U.S. Pat. No. 10,106,607 B2, hereby incorporated by reference in its entirety] and the canine light chain can comprise the constant region from kappa or lambda.

The antibodies can be engineered to include modifications to the canine framework and/or the canine frame residues within the variable domains of a parental (e.g., human) monoclonal antibody, e.g. to improve the properties of the antibody.

The construction of caninized or felinized anti-NGF monoclonal antibodies can be performed by determining a DNA sequence that encodes the heavy and light chains of canine or feline IgG, respectively. The DNA and protein sequence of the canine and feline heavy and light chains are known in the art and can be obtained by searching of the NCBI gene and protein databases.

A caninized or feline human anti-NGF antibody can be produced recombinantly by methods that are known in the field. Mammalian cell lines available as hosts for expression of the antibodies or fragments disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the heavy chain or antigen-binding portion or fragment thereof, the light chain and/or antigen-binding fragment thereof are introduced into mammalian host cells, the antibodies are produced by culturing the host cells for a period of time sufficient to allow for expression of the antibody in the host cells or, more preferably, secretion of the antibody into the culture medium in which the host cells are grown.

Antibodies can be recovered from the culture medium using standard protein purification methods. Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

Accordingly, in certain embodiments, the antibody or antigen binding fragment comprises a heavy chain constant region, e.g., a canine constant region, such as IgG-A, IgG-B, IgG-C and IgG-D canine heavy chain constant region or a variant thereof. In certain embodiments, the antibody or antigen binding fragment comprises a light chain constant region, e.g., a canine light chain constant region, such as from the lambda or kappa canine light chain region or variant thereof. By way of example, and not limitation, the canine heavy chain constant region can be from IgG-Bm and the canine light chain constant region can be from kappa.

Caninized mammalian (e.g., mouse or human) anti-human NGF antibodies that bind canine NGF of the present invention include, but are not limited to: antibodies of the present invention that comprise canine IgG-A, IgG-B, IgG-C, and IgG-D heavy chains and/or canine kappa or lambda light chains together with the anti-human NGF CDRs. Accordingly, the present invention provides caninized mouse or human antibodies of the present invention, including isolated caninized mouse or human anti-human NGF antibodies, that bind to canine NGF and that preferably also block the binding of that canine NGF to canine TrkA.

The present invention further provides caninized NGF antibodies and methods of use of the caninized antibodies of the present invention in the treatment of pain e.g., osteoarthritis in canines.

The present invention further provides full length caninized heavy chains that can be matched with corresponding light chains to make a caninized antibody. The present invention also provides full length felinized heavy chains that can be matched with corresponding light chains to make a felinized antibody. Accordingly, the present invention further provides caninized mouse or human anti-NGF antibodies (including isolated caninized human anti-human NGF antibodies) of the present invention, as well as felinized mouse or human anti-NGF antibodies (including isolated felinized human anti-human NGF antibodies) and methods of use of the antibodies of the present invention in the treatment of a condition e.g., the treatment of pain in canines and felines, respectively.

### Pharmaceutical Compositions and Administration

To prepare pharmaceutical or sterile compositions comprising the antibodies of the present invention, these antibodies can be admixed with a pharmaceutically acceptable carrier or excipient. [See, e.g., Remington's Pharmaceutical Sciences and U.S. Pharmacopeia: National Formulary, Mack Publishing Company, Easton, PA (1984)].

Formulations of therapeutic and diagnostic agents may be prepared by mixing with acceptable carriers, excipients, or stabilizers in the form of, e.g., lyophilized powders, slurries, aqueous solutions or suspensions [see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY]. In one embodiment, the antibodies of the present invention are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in canines. The dosage of such compounds lies preferably within a range of circulating concentrations that include the ED₅₀ with little or no toxicity. The dosage may vary within this range depending upon the dosage form employed and the route of administration.

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. In particular embodiments, the antibodies of the present invention can be administered by an invasive route such as by injection. In further embodiments of the invention, the antibodies of the present invention, or pharmaceutical composition thereof, is administered intravenously, subcutaneously, intramuscularly, intraarterially, or by inhalation, aerosol delivery. Administration by non-invasive routes (e.g., orally; for example, in a pill, capsule or tablet) is also within the scope of the present invention.

Compositions can be administered with medical devices known in the art. For example, a pharmaceutical composition of the invention can be administered by injection with a hypodermic needle, including, e.g., a prefilled syringe or autoinjector. The pharmaceutical compositions disclosed herein may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The pharmaceutical compositions disclosed herein may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternatively, one may administer the antibodies of the present invention in a local rather than systemic manner, often in a depot or sustained release formulation.

The administration regimen depends on several factors, including the serum or tissue turnover rate of the therapeutic antibodies, the level of symptoms, the immunogenicity of the therapeutic antibodies and the accessibility of the target cells in the biological matrix. Preferably, the administration regimen delivers sufficient therapeutic antibodies to effect improvement in the target disease/condition state, while simultaneously minimizing undesired side effects. Accordingly, the amount of biologic delivered depends in part on the particular therapeutic antibodies and the severity of the condition being treated. Guidance in selecting appropriate doses of therapeutic antibodies is available [see, e.g., Wawrzynczak Antibody Therapy, Bios Scientific Pub. Ltd, Oxfordshire, U K (1996); Kresina (ed.) Monoclonal Antibodies, Cytokines and Arthritis, Marcel Dekker, New York, NY (1991); Bach (ed.) Monoclonal Antibodies and Peptide Therapy in Autoimmune Diseases, Marcel Dekker, New York, NY (1993); Baert, et al. New Engl. J. Med. 348:601-608 (2003); Milgrom et al. New Engl. J. Med. 341:1966-1970 (1999); Slamon et al. New Engl. J. Med. 344:783-792 (2001); Beniaminovitz et al. New Engl. J. Med. 342:613-619 (2000); Ghosh et al. New Engl. J. Med. 348:24-32 (2003); Lipsky et al. New Engl. J. Med. 343:1594-1602 (2000)].

Determination of the appropriate dose is made by the veterinarian, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of the symptoms.

Antibodies provided herein may be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more [see, e.g., Yang, et al. New Engl. J. Med. 349:427-434 (2003); Herold, et al. New Engl. J. Med. 346:1692-1698 (2002); Liu, et al. J. Neurol. Neurosurg. Psych. 67:451-456 (1999); Portielji, et al. Cancer Immunol. Immunother. 52:133-144 (2003)]. Doses may also be provided to achieve a pre-determined target concentration of antibodies of the present invention in the canine's or feline's serum, such as 0.1, 0.3, 1, 3, 10, 30, 100, 300 μg/ml or more. In other embodiments, antibodies of the present invention are administered subcutaneously or intravenously, on a weekly, biweekly, “every 4 weeks,” monthly, bimonthly, or quarterly basis at 10, 20, 50, 80, 100, 200, 500, 1000 or 2500 mg/subject.

As used herein, “inhibit” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject (e.g., a canine or feline) with a disorder, condition and/or symptom, or with the potential to develop such a disorder, disease or symptom.

As used herein, the terms “therapeutically effective amount”, “therapeutically effective dose” and “effective amount” refer to an amount of antibodies of the present invention that, when administered alone or in combination with an additional therapeutic agent to a cell, tissue, or subject, e.g., canine or feline, is effective to cause a measurable improvement in one or more symptoms of a disease or condition or the progression of such disease or condition. A therapeutically effective dose further refers to that amount of the antibodies sufficient to result in at least partial amelioration of symptoms, e.g., treatment, healing, prevention or amelioration of the relevant medical condition, or an increase in rate of treatment, healing, prevention or amelioration of such conditions. When applied to a combination, a therapeutically effective dose refers to combined amounts of the active ingredients that result in the therapeutic effect, whether administered in combination, serially, or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess severity of the condition, e.g., pain.

### EXAMPLES

### Example 1

Prior Art Antibodies to Human NGF Reactive with Canine NGF

In an effort to develop a treatment for pain (e.g., for osteoarthritis) in companion animals such as dogs, cats, and horses, an investigation was undertaken to learn whether two known human or humanized antibodies to human NGF [see e.g., U.S. Pat. No. 7,601,818 B2 (fulranumab abbreviated as ful herein), U.S. Pat. No. 7,988,967 B2 (fasinumab; abbreviated as fas herein)] might also bind to NGF from dogs, cats or horses. It was found that both human/humanized monoclonal antibodies that bind to human NGF also bind to canine NGF. The set of the six prior art CDRs for these two previously disclosed antibodies are provide in Tables 1A and 1B. below.

TABLE 1A¹
AMINO ACID SEQUENCES OF THE PRIOR ART
Ful CDRS IN THE CANINIZED ANTIBODIES

| | | SEQ ID |
|---|---|---|
| CDR | Amino Acid Sequence | NO: |
| H-1 | SYSMN | 1 |
| H-2 | YISRSSHTIFYADSVKG | 2 |
| H-3 | VYSSGWHVSDYFDY | 3 |
| L-1 | RASQGISSALA | 4 |
| L-2 | DASSLES | 5 |
| L-3 | QQFNSYPLT | 6 |

TABLE 1B²
AMINO ACID SEQUENCES OF THE PRIOR ART
Fas CDRS IN THE CANINIZED ANTIBODIES

| | | SEQ ID |
|---|---|---|
| CDR | Amino Acid Sequence | NO: |
| H-1 | ELSIH | 7 |
| H-2 | GFDPEDGETIYAQKFQG | 8 |
| H-3 | IGVVTNFDN | 9 |
| L-1 | RASQAIRNDLG | 10 |
| L-2 | AAFNLQS | 11 |
| L-3 | QQYNRYPWT | 12 |

¹ The amino acid sequences in Table 1A were previously obtained and disclosed in U.S. Pat. No. 7,601,818 B2.² The amino acid sequences in Table 1B were previously obtained and disclosed in U.S. Pat. No. 7,988,967 B2.

### Example 2

### Canine NGF and Canine NGF TRKA Receptor

The amino acid sequence of the canine NGF protein is available at the national center for biotechnology information (NCBI) under accession number NP_001181879.1 [SEQ ID NO: 13]. Canine NGF-HIS-Avi protein was produced as a fusion protein of canine NGF with a C-terminal addition of 6 histidine residues and an Avi tag sequence to facilitate purification and site-specific biotinylation of the NGF protein having the amino acid sequence of SEQ ID NO: 14. The predicted amino acid sequence of the high affinity canine nerve growth factor receptor (TrkA) is available at the national center for biotechnology information (NCBI) under accession number XP_038527745. The amino acid sequence of TrkA is SEQ ID NO: 15. A cNGF-hFc Fusion protein has an amino acid sequence of SEQ ID NO: 16. For the canine NGF receptor TrkA ECD-canine Fc fusion protein, the predicted amino acid sequence of TrkA ECD was produced as a fusion protein with a C-terminal addition of the cFc from canine IgG-B. The sequence of this fusion protein is shown in SEQ ID NO: 17.

TABLE 2
CANINE NGF, CANINE NGF RECEPTOR TrkA, And RELATED FUSION PROTEINS

| | SEQ ID | |
|---|---|---|
| PROTEIN | NO: | AMINO ACID SEQUENCE |
| Canine NGF | 13 | EPHPESHVPAGHAIPHAHWTKLQHSLDTALRRARSAPAGAIAARVTGQTRNITVD |
| | | PKLFKKRRLRSPRVLFSTHPPPVAADAQDLDLEAGSTASVNRTHRSKRSSSHPVF |
| | | HRGEFSVCDSVSVWVGDKTTATDIKGKEVMVLGEVNINNSVFKQYFFETKCRDPT |
| | | PVDSGCRGIDSKHWNSYCTTTHTFVKALTMDGKQAAWRFIRIDTACVCVLSRKAG |
| | | RRA |
| Canine NGF- | 14 | EPHPESHVPAGHAIPHAHWTKLQHSLDTALRRARSAPAGAIAARVTGQTRNITVD |
| HIS-Avi | | PKLFKKRRLRSPRVLFSTHPPPVAADAQDLDLEAGSTASVNRTHRSKRSSSHPVF |
| | | HRGEFSVCDSVSVWVGDKTTATDIKGKEVMVLGEVNINNSVFKQYFFETKCRDPT |
| | | PVDSGCRGIDSKHWNSYCTTTHTFVKALTMDGKQAAWRFIRIDTACVCVLSRKAG |
| | | RRAHHHHHHGLNDIFEAQKIEWHE |
| Canine NGF | 15 | MLRGGRLGQRGGHGRAAGPGSLLAWLVLASAGAAPCPDVCCPHGPSGLRCTRAGA |
| receptor | | LQSLHRLPGVENLTELYIDNQEHLQHLDAVHLKGLGMLRDLTIVKSGLRSVAPDA |
| TrkA | | FHFTPRLRRLNLSFNALESLSWKTVQGLPLQELVLSGNPLHCSCALHWLLRWEEE |
| | | GLGGVRGQRLQCPGQGPLALLSNASCGVPVLKVQMPNASVEVGDDVLLQCQVEGQ |
| | | GLERAGWILPEVEELATVTQSGDLPSLGLTLANVTSDLNRKNVTCWAENDVGRAE |
| | | VSVQVNVSFPASVQLHEAVELHHWCIPFSVDGQPAPSLRWLFNGSVLNETSFIFT |
| | | EFLEPVANETVRHGCLRLNQPTHVNNGNYTLLAANPSGRAAAFVMAAFMDNPFEF |
| | | NPEDPIPVSFSPVDTNSTSGDPVEKKDETPFGVSVAVGLAVFACLFLSTLFLALN |
| | | KCGRRNKFGGNRAVVLAPEDGLAMSLHFMTLGGSSLSPTEGKGSGLOGHIIENPQ |
| | | YFSDACVHHIKRQDIVLKWELGEGAFGKVFLAECHNLLPEQDKMLVAVKALKEVS |
| | | ESARQDFQREAQLLTMLQHQHIVRFFGVCTEGRPLLMVFEYMRHGDLNRFLRSHG |
| | | PDAKLLAGGEDVAPGPLGLGQLLAVASQVAAGMVYLAGLHFVHRDLATRNCLVGQ |
| | | GLVVKIGDFGMSRDIYSTDYYRVGGRTMLPIRWMPPESILYRKFTTESDVWSFGV |
| | | VLWEIFTYGKQPWYQLSNTEAIECITQGRELERPRACPPEVYAIMRGCWQREPQQ |
| | | RHSIKDVHARLQALAQAPPVYLDVLG |
| cNGF-hFc | 16 | EPHPESHVPAGHAIPHAHWTKLQHSLDTALRRARSAPAGAIAARVTGQTRNITVD |
| Fusion | | PKLFKKRRLRSPRVLFSTHPPPVAADAQDLDLEAGSTASVNRTHRSKRSSSHPVF |
| protein | | HRGEFSVCDSVSVWVGDKTTATDIKGKEVMVLGEVNINNSVFKQYFFETKCRDPT |
| | | PVDSGCRGIDSKHWNSYCTTTHTFVKALTMDGKQAAWRFIRIDTACVCVLSRKAG |
| | | RRAEPKSCDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMHEAL |
| | | HNHYTQKSLSLSPGK |
| cTrkA-ECD- | 17 | AAPCPDVCCPHGPSGLRCTRAGALQSLHRLPGVENLTELYIDNQEHLQHLDAVHL |
| IgG-B Fc | | KGLGMLRDLTIVKSGLRSVAPDAFHFTPRLRRLNLSFNALESLSWKTVQGLPLQE |
| fusion | | LVLSGNPLHCSCALHWLLRWEEEGLGGVRGQRLQCPGQGPLALLSNASCGVPVLK |
| protein | | VQMPNASVEVGDDVLLQCQVEGRGLERAGWILPEVEELATVTQSGDLPSLGLTLA |
| | | NVTSDLNRKNVTCWAENDVGRAEVSVQVNVSFPASVQLHEAVELHHWCIPFSVDG |
| | | QPAPSLRWLFNGSVLNETSFIFTEFLEPVANETVRHGCLRLNQPTHVNNGNYTLL |
| | | AANPSGRAAAFVMAAFMDNPFEFNPEDPIPVSFSPVDTNSTSGDPVEKKDETPFG |
| | | VSVAVGVPKRENGRVPRPPDCPKCPAPEMLGGPSVFIFPPKPKDTLLIARTPEVT |
| | | CVVVDLDPEDPEVQISWFVDGKQMQTAKTQPREEQFNGTYRVVSVLPIGHQDWLK |
| | | GKQFTCKVNNKALPSPIERTISKARGQAHQPSVYVLPPSREELSKNTVSLTCLIK |
| | | DFFPPDIDVEWQSNGQQEPESKYRTTPPQLDEDGSYFLYSKLSVDKSRWQRGDTF |
| | | ICAVMHEALHNHYTQESLSHSPGK |

### Example 3

### Generation of Human-Canine Chimeric NGF Antibodies

Chimeric human-canine antibodies were constructed using the VH and VL sequences previously disclosed [see, Table 3 below] and then tested against canine NGF. Briefly, the VH and VL of each of a selected group of antibodies were genetically combined (fused) with the canine IgG-B heavy chain constant regions (CH1-CH3) and light chain (kappa) constant region, respectively [see Table 4 for greater detail]. The human/humanized VH and VL regions of human-canine (H-C) chimeras listed in Table 4 were transiently expressed in HEK293 cells and then purified using a Protein A column. The binding activities of the individual chimeric antibodies were tested on ELISA plates coated with canine NGF, as described in Example 4 below.

TABLE 3
VH AND VL SEQUENCES OF PRIOR ART ANTIBODIES TO HUMAN NGF

| VH AMINO ACID SEQUENCE FOR | VL AMINO ACID SEQUENCE FOR |
|---|---|
| HUMAN/HUMANIZED ANTIBODIES | HUMAN/HUMANIZED ANTIBODIES |
| hFul-VH (SEQ ID NO: 18) | hFul-VL (SEQ ID NO: 19) |
| EVQLVESGGGLVQPGGSLRLSCAASGFTLRSYSMNWVR | AIQLTQSPSSLSASVGDRVTITCRASQGISSALAW |
| QAPGKGLEWVSYISRSSHTIFYADSVKGRFTISRDNAK | YQQKPGKAPKLLIYDASSLESGVPSRFSGSGSGTD |
| NSLYLQMDSLRDEDTAMYYCARVYSSGWHVSDYFDYWG | FTLTISSLQPEDFATYYCQQFNSYPLTFGGGTKVE |
| QGILVTVSS | IK |
| hFas-VH (SEQ ID NO: 21) | hFas-VL (SEQ ID NO: 22) |
| QVQLVQSGAEVKKPGASVKVSCKVSGFTLTELSIHWVR | DIQMTQSPSSLSASAGDRVTITCRASQAIRNDLGW |
| QAPGKGLEWMGGFDPEDGETIYAQKFQGRVTMTEDTST | YQQKPGKAPKRLIYAAFNLQSGVPSRFSGSGSGTE |
| DTAYMELTSLRSEDTAVYYCSTIGVVTNFDNWGQGTLV | FTLTISSLQPEDLASYYCQQYNRYPWTFGQGTKVE |
| TVSS | IK |

TABLE 4
CHIMERIC HUMAN-CANINE ANTI-NGF

huFulVH-cIgGB (SEQ ID NO: 23)
EVQLVESGGGLVQPGGSLRLSCAASGFTLRSYSMNWVRQAPGKGLEWVSYISRSSHTIFYADSVKGRFTISRD
NAKNSLYLQMDSLRDEDTAMYYCARVYSSGWHVSDYFDYWGQGILVTVSSASTTAPSVFPLAPSCGSTSGSTV
ALACLVSGYFPEPVTVSWNSGSLTSGVHTFPSVLQSSGLYSLSSMVTVPSSRWPSETFTCNVAHPASKTKVDK
PVPKRENGRVPRPPDCPKCPAPEMLGGPSVFIFPPKPKDTLLIARTPEVTCVVVDLDPEDPEVQISWFVDGKQ
MQTAKTQPREEQFNGTYRVVSVLPIGHQDWLKGKQFTCKVNNKALPSPIERTISKARGQAHQPSVYVLPPSRE
ELSKNTVSLTCLIKDFFPPDIDVEWQSNGQQEPESKYRTTPPQLDEDGSYFLYSKLSVDKSRWQRGDTFICAV
MHEALHNHYTQESLSHSPGK
huFulVL-cCk (SEQ ID NO: 24)
AIQLTQSPSSLSASVGDRVTITCRASQGISSALAWYQQKPGKAPKLLIYDASSLESGVPSRFSGSGSGTDFTL
TISSLQPEDFATYYCQQFNSYPLTFGGGTKVEIKRNDAQPAVYLFQPSPDQLHTGSASVVCLLNSFYPKDINV
KWKVDGVIQDTGIQESVTEQDKDSTYSLSSTLTMSSTEYLSHELYSCEITHKSLPSTLIKSFQRSECQRVD
hFAS-VH-cIgGB (SEQ ID NO: 25)
QVQLVQSGAEVKKPGASVKVSCKVSGFTLTELSIHWVRQAPGKGLEWMGGFDPEDGETIYAQKFQGRVTMTED
TSTDTAYMELTSLRSEDTAVYYCSTIGVVTNFDNWGQGTLVTVSSASTTAPSVFPLAPSCGSTSGSTVALACL
VSGYFPEPVTVSWNSGSLTSGVHTFPSVLQSSGLYSLSSMVTVPSSRWPSETFTCNVAHPASKTKVDKPVPKR
ENGRVPRPPDCPKCPAPEMLGGPSVFIFPPKPKDTLLIARTPEVTCVVVDLDPEDPEVQISWFVDGKQMQTAK
TQPREEQFNGTYRVVSVLPIGHQDWLKGKQFTCKVNNKALPSPIERTISKARGQAHQPSVYVLPPSREELSKN
TVSLTCLIKDFFPPDIDVEWQSNGQQEPESKYRTTPPQLDEDGSYFLYSKLSVDKSRWQRGDTFICAVMHEAL
HNHYTQESLSHSPGK
hFas-VL-cCk (SEQ ID NO: 26)
DIQMTQSPSSLSASAGDRVTITCRASQAIRNDLGWYQQKPGKAPKRLIYAAFNLQSGVPSRFSGSGSGTEFTL
TISSLQPEDLASYYCQQYNRYPWTFGQGTKVEIKRNDAQPAVYLFQPSPDQLHTGSASVVCLLNSFYPKDINV
KWKVDGVIQDTGIQESVTEQDKDSTYSLSSTLTMSSTEYLSHELYSCEITHKSLPSTLIKSFQRSECQRVD

### Example 4

### Generation of Caninized NGF Antibodies

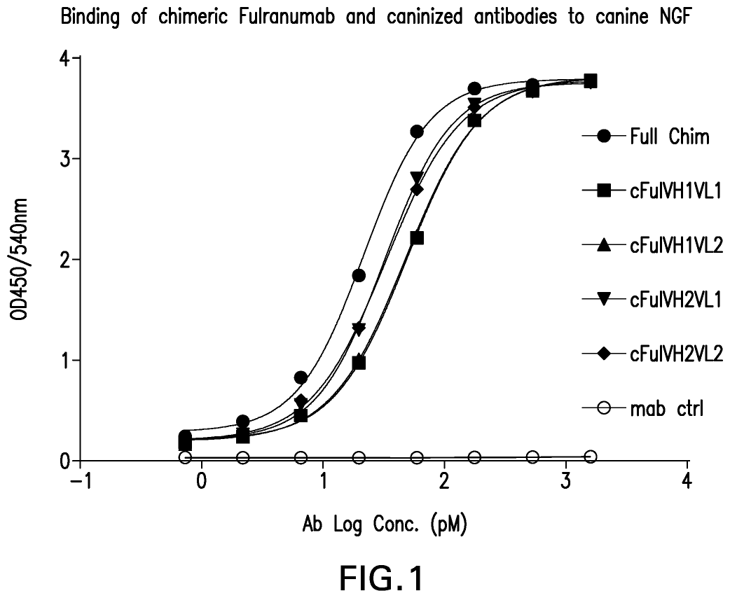

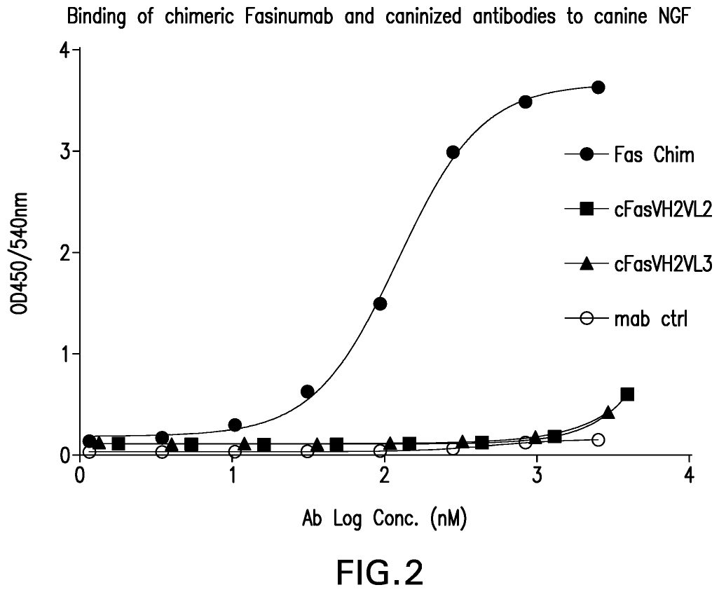

Caninized antibodies were constructed using the two sets of 6 CDRs provided in Tables 1A-1B. The binding activity of the chimeric and caninized antibodies to canine NGF was compared by ELISA (see, Example 5 below). As depicted in FIGS. 1 and 2, both chimeric antibodies show a strong affinity for canine NGF. In direct contrast, a control caninized monoclonal antibody (with the set of 6 CDRs obtained from a murine antibody raised against a non-related canine antigen) did not bind at all.

Accordingly, FIG. 1 depicts a plot of the binding of human-canine chimeric Fulranumab (Ful Chim), and the caninized variants which contain the CDRs from Fulranumab, and an isotype control mAb (mAb ctrl) as determined by ELISA. The chimeric Fulranumab bound to canine NGF had an EC50 of 22 pM, whereas the caninized variants of Fulranumab bound to canine NGF had a range of EC50 from 32-49 pM. These results demonstrate that these caninized antibodies have a strong binding affinity to canine NGF and thereby, make them suitable for development as drugs for treatment of pain in dogs.

FIG. 2 depicts a plot of the binding of human-canine chimeric Fasinumab (Fas Chim), and the caninized variants containing CDRs from Fasinumab and isotype control mAb (mAb ctrl) as determined by ELISA. Surprisingly however, whereas the chimeric Fasinumab bound to canine NGF with an EC50 of 122 nM, the binding affinity for canine NGF of the corresponding caninized variants containing the CDRs from Fasinumab was too low to measure. This makes the caninized Fasinumab antibodies unsuitable for development for the treatment of pain in dogs. This demonstrates that is unpredictable whether a caninized antibody encoding a set of CDRs from a given antibody to human NGF would also bind to the canine NGF, even when the corresponding human-canine chimera does bind well.

TABLE 5
VH AND VL AMINO ACID SEQUENCES OF CANINIZED
ANTIBODIES TO HUMAN AND CANINE NGF.

cFul_VH1 (SEQ ID NO: 27)
EVQLVESGGDLVKPGGSLRLSCVASGFTFSSYSMNWIRQAPGKGLQWVSYISRSSHTIFYADSVKGRFTISRD
NAKNTLYLQMNSLRDEDTAVYYCARVYSSGWHVSDYFDYWGQGTLVTVSS
cFul_VH2 (SEQ ID NO: 28)
EVQLVESGGDLVKPGGSLRLSCVASGFTLRSYSMNWIRQAPGKGLQWVSYISRSSHTIFYADSVKGRFTISRD
NAKNTLYLQMDSLRDEDTAVYYCARVYSSGWHVSDYFDYWGQGILVTVSS
cFul-VL1 (SEQ ID NO: 29)
EIVMTQSPASLSLSQEEKVTITCRASQGISSALAWYQQKPGQAPKLLIYDASSLESGVPSRFSGSGSGTDFSF
TISSLEPEDVAVYYCQQFNSYPLTFGQGTKVEIK
cFul-VL2 (SEQ ID NO: 30)
EIQLTQSPASLSLSQEEKVTITCRASQGISSALAWYQQKPGQAPKLLIYDASSLESGVPSRFSGSGSGTDFSL
TISSLEPEDFAVYYCQQFNSYPLTFGGGTKVEIK
cFas-VH1 (SEQ ID NO: 31)
EVQLVQSGAEVKKPGASVKVSCKTSGYTFIELSIHWVRQAPGAGLDWMGGFDPEDGETIYAQKFQGRVTLTAD
TSTSTAYMELSSLRAGDIAVYYCARIGVVTNFDNWGQGTLVTVSS
cFas-VH2 (SEQ ID NO: 32)
EVQLVQSGAEVKKPGASVKVSCKVSGYTLTELSIHWVRQAPGKGLDWMGGFDPEDGETIYAQKFQGRVTLTED
TSTDTAYMELSSLRAGDIAVYYCSTIGVVTNFDNWGQGTLVTVSS
cFas-VL1 (SEQ ID NO: 33)
EIVMTQSPASLSLSQEEKVTITCRASQAIRNDLGWYQQKPGQAPKLLIYAAFNLQSGVPSRFSGSGSGTDFSF
TISSLEPEDVAVYYCQQYNRYPWTFGQGTKLEIK
cFas-VL2 (SEQ ID NO: 34)
DIVMTQTPLSLSVSPGETASISCRASQAIRNDLGWFRQKPGQSPQRLIYAAFNLQSGVPDRFSGSGSGTDFTL
RISRVEADDTGVYYCQQYNRYPWTFGQGTKLEIK
cFas-VL3 (SEQ ID NO: 35)
DIVMTQTPLSLSVSPGETASISCRASQAIRNDLGWFRQKPGKSPKRLIYAAFNLQSGVPDRFSGSGSGTDFTL
TISSVEADDTGVYYCQQYNRYPWTFGQGTKLEIK
CDRs are underlined

HEAVY AND LIGHT CHAINS OF CANINIZED ANTIBODIES
cFulVH1-cIgGB
(SEQ ID NO: 36)
EVQLVESGGDLVKPGGSLRLSCVASGFTFSSYSMNWIRQAPGKGLQWVSYISRSSHTIFYADSVKGRFTISRDNA
KNTLYLQMNSLRDEDTAVYYCARVYSSGWHVSDYFDYWGQGTLVTVSSASTTAPSVFPLAPSCGSTSGSTVALAC
LVSGYFPEPVTVSWNSGSLTSGVHTFPSVLQSSGLYSLSSMVTVPSSRWPSETFTCNVAHPASKTKVDKPVPKRE
NGRVPRPPDCPKCPAPEMLGGPSVFIFPPKPKDTLLIARTPEVTCVVVDLDPEDPEVQISWFVDGKQMQTAKTQP
REEQFNGTYRVVSVLPIGHQDWLKGKQFTCKVNNKALPSPIERTISKARGQAHQPSVYVLPPSREELSKNTVSLT
CLIKDFFPPDIDVEWQSNGQQEPESKYRTTPPQLDEDGSYFLYSKLSVDKSRWQRGDTFICAVMHEALHNHYTQE
SLSHSPGK
cFulVH2-cIgGB
(SEQ ID NO: 37)
EVQLVESGGDLVKPGGSLRLSCVASGFTLRSYSMNWIRQAPGKGLQWVSYISRSSHTIFYADSVKGRFTISRDNA
KNTLYLQMDSLRDEDTAVYYCARVYSSGWHVSDYFDYWGQGILVTVSSASTTAPSVFPLAPSCGSTSGSTVALAC
LVSGYFPEPVTVSWNSGSLTSGVHTFPSVLQSSGLYSLSSMVTVPSSRWPSETFTCNVAHPASKTKVDKPVPKRE
NGRVPRPPDCPKCPAPEMLGGPSVFIFPPKPKDTLLIARTPEVTCVVVDLDPEDPEVQISWFVDGKQMQTAKTQP
REEQFNGTYRVVSVLPIGHQDWLKGKQFTCKVNNKALPSPIERTISKARGQAHQPSVYVLPPSREELSKNTVSLT
CLIKDFFPPDIDVEWQSNGQQEPESKYRTTPPQLDEDGSYFLYSKLSVDKSRWQRGDTFICAVMHEALHNHYTQE
SLSHSPGK
cFulVL1-cCk
(SEQ ID NO: 38)
EIVMTQSPASLSLSQEEKVTITCRASQGISSALAWYQQKPGQAPKLLIYDASSLESGVPSRFSGSGSGTDFSFTI
SSLEPEDVAVYYCQQFNSYPLTFGQGTKVEIKRNDAQPAVYLFQPSPDQLHTGSASVVCLLNSFYPKDINVKWKV
DGVIQDTGIQESVTEQDKDSTYSLSSTLTMSSTEYLSHELYSCEITHKSLPSTLIKSFQRSECQRVD
cFulVL2-cCk
(SEQ ID NO: 39)
EIQLTQSPASLSLSQEEKVTITCRASQGISSALAWYQQKPGQAPKLLIYDASSLESGVPSRFSGSGSGTDFSLTI
SSLEPEDFAVYYCQQFNSYPLTFGGGTKVEIKRNDAQPAVYLFQPSPDQLHTGSASVVCLLNSFYPKDINVKWKV
DGVIQDTGIQESVTEQDKDSTYSLSSTLTMSSTEYLSHELYSCEITHKSLPSTLIKSFQRSECQRVD
cFAS-VH1-cIgGB
(SEQ ID NO: 40)
EVQLVQSGAEVKKPGASVKVSCKTSGYTFIELSIHWVRQAPGAGLDWMGGFDPEDGETIYAQKFQGRVTLTADTS
TSTAYMELSSLRAGDIAVYYCARIGVVTNFDNWGQGTLVTVSSASTTAPSVFPLAPSCGSTSGSTVALACLVSGY
FPEPVTVSWNSGSLTSGVHTFPSVLQSSGLYSLSSMVTVPSSRWPSETFTCNVAHPASKTKVDKPVPKRENGRVP
RPPDCPKCPAPEMLGGPSVFIFPPKPKDTLLIARTPEVTCVVVDLDPEDPEVQISWFVDGKQMQTAKTQPREEQF
NGTYRVVSVLPIGHQDWLKGKQFTCKVNNKALPSPIERTISKARGQAHQPSVYVLPPSREELSKNTVSLTCLIKD
FFPPDIDVEWQSNGQQEPESKYRTTPPQLDEDGSYFLYSKLSVDKSRWQRGDTFICAVMHEALHNHYTQESLSHS
PGK
cFAS-VH2-cIgGB
(SEQ ID NO: 41)
EVQLVQSGAEVKKPGASVKVSCKVSGYTLTELSIHWVRQAPGKGLDWMGGFDPEDGETIYAQKFQGRVTLTEDTS
TDTAYMELSSLRAGDIAVYYCSTIGVVTNFDNWGQGTLVTVSSASTTAPSVFPLAPSCGSTSGSTVALACLVSGY
FPEPVTVSWNSGSLTSGVHTFPSVLQSSGLYSLSSMVTVPSSRWPSETFTCNVAHPASKTKVDKPVPKRENGRVP
RPPDCPKCPAPEMLGGPSVFIFPPKPKDTLLIARTPEVTCVVVDLDPEDPEVQISWFVDGKQMQTAKTQPREEQF
NGTYRVVSVLPIGHQDWLKGKQFTCKVNNKALPSPIERTISKARGQAHQPSVYVLPPSREELSKNTVSLTCLIKD
FFPPDIDVEWQSNGQQEPESKYRTTPPQLDEDGSYFLYSKLSVDKSRWQRGDTFICAVMHEALHNHYTQESLSHS
PGK
cFAS-VL1-cCk
(SEQ ID NO: 42)
EIVMTQSPASLSLSQEEKVTITCRASQAIRNDLGWYQQKPGQAPKLLIYAAFNLQSGVPSRFSGSGSGTDFSFTI
SSLEPEDVAVYYCQQYNRYPWTFGQGTKLEIKRNDAQPAVYLFQPSPDQLHTGSASVVCLLNSFYPKDINVKWKV
DGVIQDTGIQESVTEQDKDSTYSLSSTLTMSSTEYLSHELYSCEITHKSLPSTLIKSFQRSECQRVD
cFAS-VL2-cCk
(SEQ ID NO: 43)
DIVMTQTPLSLSVSPGETASISCRASQAIRNDLGWFRQKPGQSPQRLIYAAFNLQSGVPDRFSGSGSGTDFTLRI
SRVEADDTGVYYCQQYNRYPWTFGQGTKLEIKRNDAQPAVYLFQPSPDQLHTGSASVVCLLNSFYPKDINVKWKV
DGVIQDTGIQESVTEQDKDSTYSLSSTLTMSSTEYLSHELYSCEITHKSLPSTLIKSFQRSECQRVD
cFAS-VL3-cCk
(SEQ ID NO: 44)
DIVMTQTPLSLSVSPGETASISCRASQAIRNDLGWFRQKPGKSPKRLIYAAFNLQSGVPDRFSGSGSGTDFTLTI
SSVEADDTGVYYCQQYNRYPWTFGQGTKLEIKRNDAQPAVYLFQPSPDQLHTGSASVVCLLNSFYPKDINVKWKV
DGVIQDTGIQESVTEQDKDSTYSLSSTLTMSSTEYLSHELYSCEITHKSLPSTLIKSFQRSECQRVD

### Example 5

### Binding of Chimeric and Caninized Anti-Human NGF Antibodies to Canine NGF

### Methods

The binding of chimeric and caninized antibodies to canine NGF was determined by ELISA as follows:
1. Coat 100 ng/well canine NGF in an immunoplate and incubate the plate at 4° C. overnight.
2. Wash the plate 3 times by PBS with 0.05% Tween 20 (PBST).
3. Block the plate by 0.5% BSA in PBS for 45-60 min at room temperature.
4. Wash the plate 3 times by PBST.
5. Make 3-fold dilution the antibodies in each column or row of dilution plate.
6. Transfer the diluted antibodies into each column or row of the immunoplate, and incubate the plate for 45-60 min at room temperature.
7. Wash the plate 3 times by PBST.
8. Add 1:2000 diluted horseradish peroxidase labeled anti-dog IgG Fc into each well of the plate and incubate the plate for 45-60 min at room temperature.
9. Wash the plate 3 times by PBST.
10. Add TMB Substrate into each well of the plate and incubate the plate for 10 to 15 min at room temperature for color development.
11. Add 100 μL of 1.5 M phosphoric acid into each well to stop the reaction.
12. Read the plate at 450 nm with 540 nm reference wavelength.

### Results

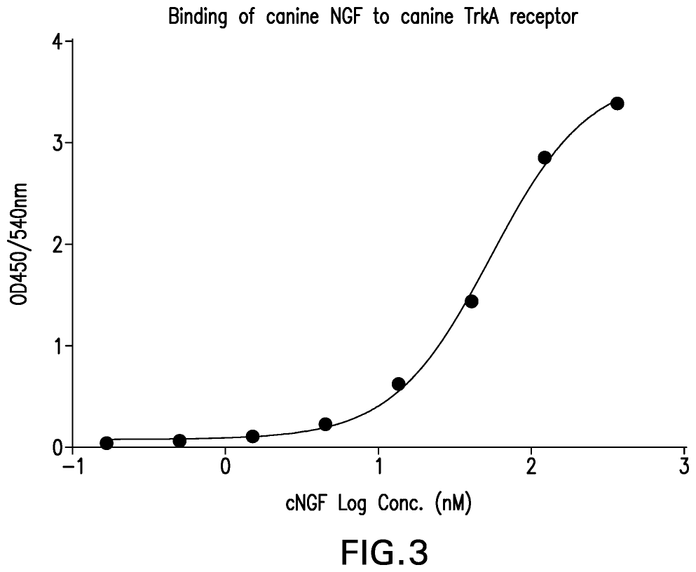

FIG. 3 shows the binding of canine NGF to the canine TrkA receptor. The binding of canine NGF to canine NGF receptor (TrkA) was determined by ELISA in order to develop an assay to measure the ability of caninized anti-canine NGF antibodies to block the binding of canine NGF to its TrkA receptor. As shown in FIG. 3, canine NGF binds to its canine TrkA receptor in a dose dependent manner and with an EC50 of 54 nM.

### Example 6

### Blocking Activity of the Chimeric and Caninized Antibodies

### Methods

The chimeric and caninized anti-NGF antibodies were tested for blocking the binding of canine NGF to the canine NGF receptor (TrkA) as follows:
1. Coat 100 ng/well canine TrkA-IgGBFc fusion protein in an immuno-plate and incubate the plate at 4° C. overnight.
2. Wash the plate 3 times by PBS with 0.05% Tween 20 (PBST).
3. Block the plate by 0.5% BSA in PBS for 45-60 min at room temperature.
4. Wash the plate 3 times by PBST.
5. Make a 3-fold dilution of the antibodies in each column or row of dilution plate, and then add 100 ng/well biotinylated canine NGF and mix with the antibodies.
6. Transfer the diluted antibodies and canine NGF mixture into each column or row of the immunoplate, and incubate the plate for 45-60 min at room temperature.
7. Wash the plate 3 times by PBST.
8. Add 1:2000 diluted horseradish peroxidase conjugated streptavidin into each well of the plate and incubate the plate for 45-60 min at room temperature.
9. Wash the plate 3 times by PBST.
10. Add TMB Substrate into each well of the plate and incubate the plate for 10 to 15 min at room temperature for color development.
11. Add 100 μL of 1.5 M phosphoric acid into each well to stop the reaction.
12. Read the plate at 450 nm with 540 nm reference wavelength.

### Results

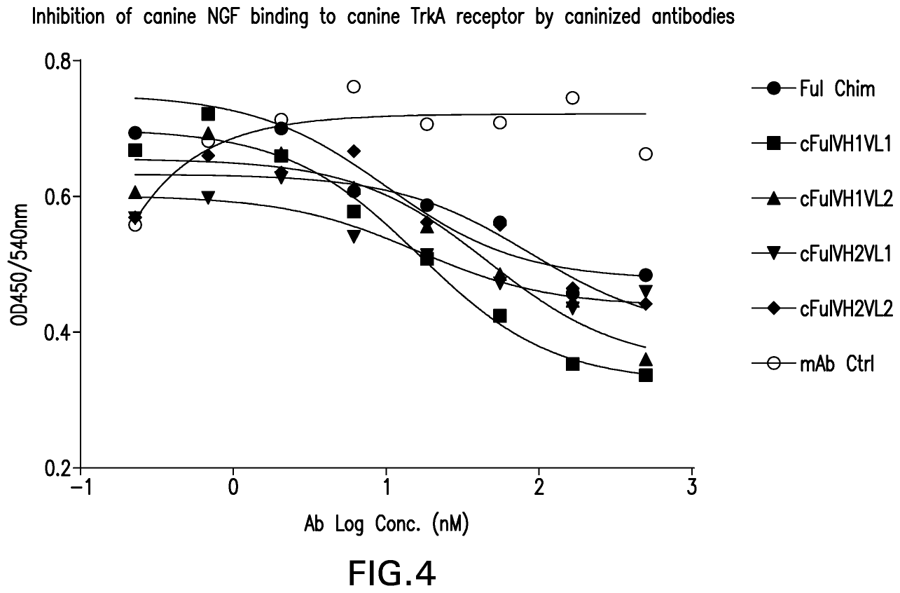

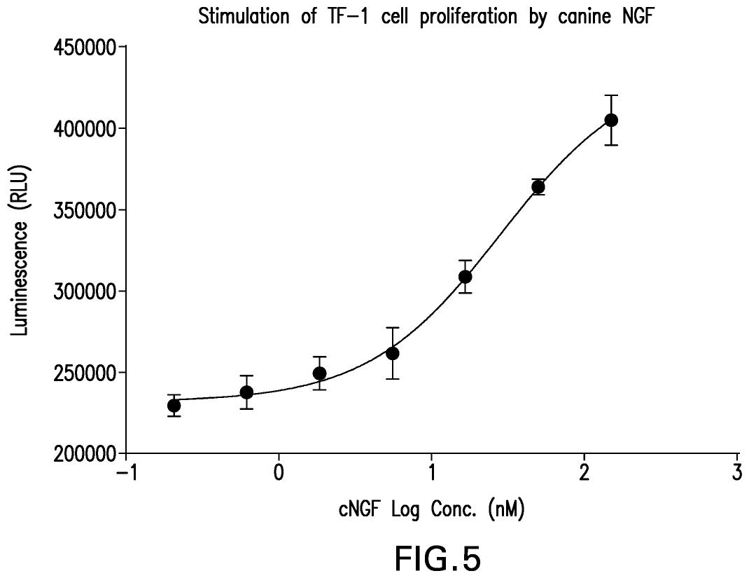

FIG. 4 depicts the inhibition of canine NGF binding to canine TrkA receptor by caninized antibodies and the corresponding human-canine chimeric Fulranumab. The ability of human-canine chimeric Fulranumab (Ful Chim), and the caninized variants containing CDRs from Fulranumab to block the binding of canine NGF to its TrkA receptor was determined by ELISA. As shown, the chimeric Fulranumab and the caninized variants containing CDRs from Fulranumab both specifically and in a dose dependent manner inhibited the binding of canine NGF to its TrkA receptor with a range of IC50 from 10-87 nM. In direct contrast, the isotype control mAb (mAb ctrl) did not. These results indicate that the caninized antibodies are suitable for development for the treatment of pain in dogs.

### Example 7

### Inhibition of Canine NGF Bioactivity in TF-1 Cells

### Materials and Methods

### TF-1 Cell-Based Assay

TF-1 is a human erythroleukemic cell line that expresses human TrkA and proliferateS in response to NGF from various species. The effect of canine NGF on proliferation of TF-1 cells and the ability of chimeric and caninized anti-NGF antibodies to block proliferation of TF-1 cells were assessed as follows:

### Materials

### TF1 Cell Line (CRL-2003)

Growth medium: RPMI-1640 (ThermoFisher CAT #11875-085), 10% FBS and 2 ng/mL rhGM-CSF (R&D, 7954-GM)
Assay Medium: RPMI-1640 (ThermoFisher CAT #11875-085) with 10% FBS
CELLTITER-GLO® One Solution Assay (Promega cat #G8461)

### Methods

### TF-1 Cell Culture:

1. Cells are incubated in T75 flask in a cell culture incubator at 37° C. with 5% CO₂ and >80% relative humidity. Cells are Passaged every 3-4 days when seeded as 4-8×10⁴ cells/mL in growth medium.
2. Passage the cells one day before cell proliferation assay conducted.

### TF-1 Cell Proliferation Mediated by Canine Beta-NGF Assay:

1. Add 50 μL of assay medium to each well of 96-well plate.
2. Prepare 900 nM recombinant canine beta-NGF (cNGF) in assay medium. Add 25 μL of the cNGF to the first wells. In duplicate, 3-fold dilute across the plate and discard final 25 μL volume.
3. Harvest the TF-1 cells and wash 3× in assay medium. Resuspend the cells in assay medium to a concentration of 0.5-1×10⁶ cells/mL.
4. Add 50 μL of the TF-1 cells to each well of the assay plate.
5. Incubate the plate for 48 hours (±8 hours) in a cell culture incubator at 37° C. with
5% CO₂ and >80% relative humidity.
6. Add 100 μL/well of CELLTITER-GLO ONE SOLUTION ASSAY into the plate. Mix contents for 2 minutes on an orbital shaker and incubate at room temperature for 15 minutes (±5 minutes).
7. Measure luminescence intensity by plate reader.
   Inhibition of cNGF Mediated TF-1 Cell Proliferation by Anti-cNGF Antibodies:
1. Add 50 μL of assay medium to each well of 96-well plate.
2. Prepare 1800 nM antibody in assay medium. Add 25 μL of the antibody to the first wells. In duplicate, 3-fold dilute across the plate and discard final 25 μL volume. mAb iso-control, wells with assay medium and cell only are included
3. Prepare 60 nM cNGF in assay medium. Mix equal volume of the cNGF to the diluted antibody.
4. Harvest the TF-1 cells and wash 3× in assay medium. Resuspend the cells in assay medium to a concentration of 0.5-1×10⁶ cells/mL.
5. Add 50 μL of the TF-1 cells to each well of a new 96-well plate. Transfer 50 μL of the mixed cNGF/antibody to each well of the cell plate.
6. Incubate the plate for 48 hours (±8 hours) in a cell culture incubator at 37° C. with 5% CO₂ and >80% relative.
7. Add 100 μL/well of CELLTITER-GLO ONE SOLUTION ASSAY into the plate. Mix contents for 2 minutes on an orbital shaker and incubate at room temperature for 15 minutes (±5 minutes).
8. Measure luminescence intensity by plate reader.

### Results

FIG. 5 shows the stimulation of TF-1 cell proliferation by canine NGF. The ability of canine NGF to stimulate proliferation of TF-1 cells was determined by a bioassay in order to develop an assay to measure the ability of caninized anti-canine NGF antibodies to block downstream signaling and inhibit cell proliferation induced by the binding of canine NGF to the TrkA receptor on TF-1 cells. As shown, canine NGF binds in a dose dependent manner with EC50 of 28 nM to endogenous TrkA receptor expressed by to TF-1 cells and stimulates TF-1 cell proliferation. This result shows that the TF-1 cell-based assay can be used to test blocking activity of anti-canine NGF antibodies.

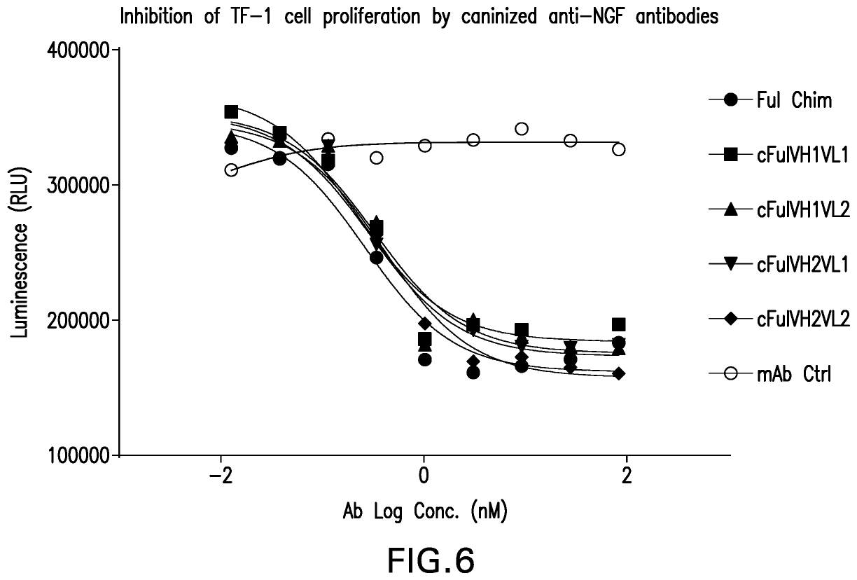

FIG. 6 shows the inhibition of TF-1 cell proliferation by caninized anti-NGF antibodies. The ability of human-canine chimeric Fulranumab (Ful Chim), the caninized variants containing CDRS from Fulranumab identified in FIG. 6, and isotype control mAb (mAb ctrl) to block TF-1 cell proliferation was determined in a bioassay with TF-1 cells. As shown, the chimeric and the caninized variants containing CDRs from Fulranumab specifically and in a dose dependent manner inhibited TF-1 cell proliferations with a range of IC50 from 0.26-0.4 nM. These results demonstrate that the caninized antibodies are suitable for development for treatment of pain in dogs.

### Example 8

Felinized Human NGF Antibodies and their Inhibition of Feline NGF Bioactivity in TF-1 Cells

### Felinized Human NGF Antibodies

### Materials and Methods

### Feline IgG Heavy Chain Constant Regions:

The constant regions for feline IgG subclasses and their hinge regions have been described previously [Strietzel et al., Vet Immunol & Immunpathol., 158:214-223 (2014)]. The amino acid sequences of the feline heavy chain constant regions are available from Genbank databases [IgG1a accession number BAA32229.1, IgG1b accession number BAA32230.1 and IgG2 accession number AHH34165.1]. IgG1a and IgG1b appear to be allelic variants, and both bind strongly to human C1q suggesting that they may be able to activate complement. In contrast feline IgG2 has a divergent sequence especially in the hinge region and has negligible binding to C1q. In order to provide IgG1a or IgG1b heavy chain constant regions lacking effector functions, as shown below, the aspartic acid (D) amino acid at position 151 is substituted with an alanine, and the asparagine (N) amino acid at position 183 is substituted with an alanine (A) in the unsubstituted amino acid sequences SEQ ID NOs: 54 and 55 shown below. The alignment of the feline IgG subclasses is:

Feline IgG1a

| | |
|---|---|
| 1 | ASTTAPSVFP LAPSCGTTSG ATVALACLVL GYFPEPVTVS |
| | WNSGALTSGV HTFPAVLQAS |

Feline IgG1b

| | |
|---|---|
| 1 | ASTTAPSVFP LAPSCGTTSG ATVALACLVL GYFPEPVTVS |
| | WNSGALTSGV HTFPAVLQAS |

Feline IgG2

| | |
|---|---|
| 1 | ASTTASSVFP LAPSCGTTSG ATVALACLVL GYFPEPVTVS |
| | WNSGALTSGV HTFPSVLQAS |

Feline IgG1a

| | |
|---|---|
| 61 | GLYSLSSMVT VPSSRWLSDT FTCNVAHPPS NTKVDKTVRK |
| | TDHPPGPKPC DCPKCPPPEM |

Feline IgG1b

| | |
|---|---|
| 61 | GLYSLSSMVT VPSSRWLSDT FTCNVAHPPS NTKVDKTVRK |
| | TDHPPGPKPC DCPKCPPPEM |

Feline IgG2

| | |
|---|---|
| 61 | GLYSLSSMVT VPSSRWLSDT FTCNVAHRPS STKVDKTVPK |
| | TASTIESKTG EGPKCPVPEI |

Feline IgG1a

| | |
|---|---|
| 121 | LGGPSIFIFP PKPKDTLSIS RTPEVTCLVV DLGPDDSDVQ |
| | ITWFVDNTQV YTAKTSPREE |

Feline IgG1b

| | |
|---|---|
| 121 | LGGPSIFIFP PKPKDTLSIS RTPEVTCLVV DLGPDDSDVQ |
| | ITWFVDNTQV YTAKTSPREE |

Feline IgG2

| | |
|---|---|
| 121 | PGAPSVFIFP PKPKDTLSIS RTPEVTCLVV DLGPDDSNVQ |
| | ITWFVDNTEM HTAKTRPREE |

Feline IgG1a

| | |
|---|---|
| 181 | QFNSTYRVVS VLPILHQDWL KGKEFKCKVN SKSLPSPIER |
| | TISKAKGQPH EPQVYVLPPA |

Feline IgG1b

| | |
|---|---|
| 181 | QFNSTYRVVS VLPILHQDWL KGKEFKCKVN SKSLPSPIER |
| | TISKDKGQPH EPQVYVLPPA |

Feline IgG2

| | |
|---|---|
| 181 | QFNSTYRVVS VLPILHQDWL KGKEFKCKVN SKSLPSAMER |
| | TISKAKGQPH EPQVYVLPPT |

Feline IgG1a

| | |
|---|---|
| 241 | QEELSRNKVS VTCLIKSFHP PDIAVEWEIT GQPEPENNYR |
| | TTPPQLDSDG TYFVYSKLSV |

Feline IgG1b

| | |
|---|---|
| 241 | QEELSRNKVS VTCLIEGFYP SDIAVEWEIT GQPEPENNYR |
| | TTPPQLDSDG TYFLYSRLSV |

Feline IgG2

| | |
|---|---|
| 241 | QEELSENKVS VTCLIKGFHP PDIAVEWEIT GQPEPENNYQ |
| | TTPPQLDSDG TYFLYSRLSV |

Feline IgG1a
[SEQ ID NO: 54]

| | |
|---|---|
| 301 | DRSHWQRGNT YTCSVSHEAL HSHHTQKSLT QSPGK |

Feline IgG1b
[SEQ ID NO: 55]

| | |
|---|---|
| 301 | DRSRWQRGNT YTCSVSHEAL HSHHTQKSLT QSPGK |

Feline IgG2
[SEQ ID NO: 56]

| | |
|---|---|
| 301 | DRSHWQRGNT YTCSVSHEAL HSHHTQKSLT QSPGK |

TABLE 6
VH and VL amino acid sequences of Felinized
antibodies to feline NGF

fFul_Vh1 [SEQ ID NO: 57]
DVQLVESGGDLVQPGGSLRLTCAASGFTLRSYSMNWVRQAPGKGLQWVSY
ISRSSHTIFYADSVKGRFTISRDNAKNTLYLQMNSLKTEDTATYYCAKVY
SSGWHVSDYFDYWGQGILVTVSS
fFul_Vh2 [SEQ ID NO: 58]
EVQLVESGGDLVQPGGSLRLTCVASGFTFSSYSMNWVRQAPGKGLQWVSY
ISRSSHTIFYADSVKGRFTISRDNAKNTLYLQMDSLKDEDTATYYCARVY
SSGWHVSDYFDYWGQGILVTVSS
fFul_Vh3 [SEQ ID NO: 59]
EVQLVESEGDLVQPGGSLRLTCAASGFTLRSYSMNWVRQAPGKGLQWVSY
ISRSSHTIFYADSVKGRFTISRDNAKNTLYLQMNNLKTEDTATYYCAKVY
SSGWHVSDYFDYWGQGILVTVSS
fFul_Vh4 [SEQ ID NO: 60]
EVQLVESEGDLVQPGGSLRLTCVASGFTFSSYSMNWVRQAPGKGLQWVSY
ISRSSHTIFYADSVKGRFTISRDNAKNTLYLQMDSLKDEDTATYYCARVY
SSGWHVSDYFDYWGQGILVTVSS
fFul-VL1 [SEQ ID NO: 61]
EIQLTQSPSSLSASPGDRVTITCRASQGISSALAWYQQKPGKVPKLLIYD
ASSLESGVPSRFSGSGSGTDFTLTISSLEPEDAATYYCQQFNSYPLTFGQ
GTKLEIK
fFul-VL2 [SEQ ID NO: 62]
AITLTQSPGSLAGSPGQQVTITCRASQGISSALAWYQQKPGQHPKLLIYD
ASSLESGVPDRFSGSGSGTDFTLTISNLQAEDVASYYCQQFNSYPLTFGQ
GTKLEIK

The CDRs are underlined

### List of Heavy and Light Chains to Make Human-Feline Chimeric and Felinized Antibodies

hFulVh-fIgG1am4
[SEQ ID NO: 63]
EVQLVESGGGLVQPGGSLRLSCAASGFTLRSYSMNWVRQAPGKGLEWVSYISRSSHTIFYADSVKGRFTISRDNA
KNSLYLQMDSLRDEDTAMYYCARVYSSGWHVSDYFDYWGQGILVTVSSASTTAPSVFPLAPSCGTTSGATVALAC
LVLGYFPEPVTVSWNSGALTSGVHTFPAVLQASGLYSLSSMVTVPSSRWLSDTFTCNVAHPPSNTKVDKTVRKTD
HPPGPKPCDCPKCPPPEMLGGPSIFIFPPKPKDTLSISRTPEVTCLVVALGPDDSDVQITWFVDNTQVYTAKTSP
REEQFASTYRVVSVLPILHQDWLKGKEFKCKVNSKSLPSPIERTISKAKGQPHEPQVYVLPPAQEELSRNKVSVT
CLIKSFHPPDIAVEWEITGQPEPENNYRTTPPQLDSDGTYFVYSKLSVDRSHWQRGNTYTCSVSHEALHSHHTQK
SLTQSPGK
fFulVH1-fIgG1am4
[SEQ ID NO: 64]
DVQLVESGGDLVQPGGSLRLTCAASGFTLRSYSMNWVRQAPGKGLQWVSYISRSSHTIFYADSVKGRFTISRDNA
KNTLYLQMNSLKTEDTATYYCAKVYSSGWHVSDYFDYWGQGILVTVSSASTTAPSVFPLAPSCGTTSGATVALAC
LVLGYFPEPVTVSWNSGALTSGVHTFPAVLQASGLYSLSSMVTVPSSRWLSDTFTCNVAHPPSNTKVDKTVRKTD
HPPGPKPCDCPKCPPPEMLGGPSIFIFPPKPKDTLSISRTPEVTCLVVALGPDDSDVQITWFVDNTQVYTAKTSP
REEQFASTYRVVSVLPILHQDWLKGKEFKCKVNSKSLPSPIERTISKAKGQPHEPQVYVLPPAQEELSRNKVSVT
CLIKSFHPPDIAVEWEITGQPEPENNYRTTPPQLDSDGTYFVYSKLSVDRSHWQRGNTYTCSVSHEALHSHHTQK
SLTQSPGK
fFulVH2-fIgG1am4
[SEQ ID NO: 65]
EVQLVESGGDLVQPGGSLRLTCVASGFTFSSYSMNWVRQAPGKGLQWVSYISRSSHTIFYADSVKGRFTISRDNA
KNTLYLQMDSLKDEDTATYYCARVYSSGWHVSDYFDYWGQGILVTVSSASTTAPSVFPLAPSCGTTSGATVALAC
LVLGYFPEPVTVSWNSGALTSGVHTFPAVLQASGLYSLSSMVTVPSSRWLSDTFTCNVAHPPSNTKVDKTVRKTD
HPPGPKPCDCPKCPPPEMLGGPSIFIFPPKPKDTLSISRTPEVTCLVVALGPDDSDVQITWFVDNTQVYTAKTSP
REEQFASTYRVVSVLPILHQDWLKGKEFKCKVNSKSLPSPIERTISKAKGQPHEPQVYVLPPAQEELSRNKVSVT
CLIKSFHPPDIAVEWEITGQPEPENNYRTTPPQLDSDGTYFVYSKLSVDRSHWQRGNTYTCSVSHEALHSHHTQK
SLTQSPGK
fFulVH3-fIgG1am4
[SEQ ID NO: 66]
EVQLVESEGDLVQPGGSLRLTCAASGFTLRSYSMNWVRQAPGKGLQWVSYISRSSHTIFYADSVKGRFTISRDNA
KNTLYLQMNNLKTEDTATYYCAKVYSSGWHVSDYFDYWGQGILVTVSSASTTAPSVFPLAPSCGTTSGATVALAC
LVLGYFPEPVTVSWNSGALTSGVHTFPAVLQASGLYSLSSMVTVPSSRWLSDTFTCNVAHPPSNTKVDKTVRKTD
HPPGPKPCDCPKCPPPEMLGGPSIFIFPPKPKDTLSISRTPEVTCLVVALGPDDSDVQITWFVDNTQVYTAKTSP
REEQFASTYRVVSVLPILHQDWLKGKEFKCKVNSKSLPSPIERTISKAKGQPHEPQVYVLPPAQEELSRNKVSVT
CLIKSFHPPDIAVEWEITGQPEPENNYRTTPPQLDSDGTYFVYSKLSVDRSHWQRGNTYTCSVSHEALHSHHTQK
SLTQSPGK
fFulVH4-fIgG1am4
[SEQ ID NO: 67]
EVQLVESEGDLVQPGGSLRLTCVASGFTFSSYSMNWVRQAPGKGLQWVSYISRSSHTIFYADSVKGRFTISRDNA
KNTLYLQMDSLKDEDTATYYCARVYSSGWHVSDYFDYWGQGILVTVSSASTTAPSVFPLAPSCGTTSGATVALAC
LVLGYFPEPVTVSWNSGALTSGVHTFPAVLQASGLYSLSSMVTVPSSRWLSDTFTCNVAHPPSNTKVDKTVRKTD
HPPGPKPCDCPKCPPPEMLGGPSIFIFPPKPKDTLSISRTPEVTCLVVALGPDDSDVQITWFVDNTQVYTAKTSP
REEQFASTYRVVSVLPILHQDWLKGKEFKCKVNSKSLPSPIERTISKAKGQPHEPQVYVLPPAQEELSRNKVSVT
CLIKSFHPPDIAVEWEITGQPEPENNYRTTPPQLDSDGTYFVYSKLSVDRSHWQRGNTYTCSVSHEALHSHHTQK
SLTQSPGK
fFulVL1-fCk
[SEQ ID NO: 68]
EIQLTQSPSSLSASPGDRVTITCRASQGISSALAWYQQKPGKVPKLLIYDASSLESGVPSRFSGSGSGTDFTLTI
SSLEPEDAATYYCQQFNSYPLTFGQGTKLEIKRSDAQPSVFLFQPSLDELHTGSASIVCILNDFYPKEVNVKWKV
DGVVQNKGIQESTTEQNSKDSTYSLSSTLTMSSTEYQSHEKFSCEVTHKSLASTLVKSFNRSECQRE
fFulVL2-fCk
[SEQ ID NO: 69]
AITLTQSPGSLAGSPGQQVTITCRASQGISSALAWYQQKPGQHPKLLIYDASSLESGVPDRFSGSGSGTDFTLTI
SNLQAEDVASYYCQQFNSYPLTFGQGTKLEIKRSDAQPSVFLFQPSLDELHTGSASIVCILNDFYPKEVNVKWKV
DGVVQNKGIQESTTEQNSKDSTYSLSSTLTMSSTEYQSHEKFSCEVTHKSLASTLVKSFNRSECORE
hFulV1-fCk
[SEQ ID NO: 70]
AIQLTQSPSSLSASVGDRVTITCRASQGISSALAWYQQKPGKAPKLLIYDASSLESGVPSRFSGSGSGTDFTLTI
SSLQPEDFATYYCQQFNSYPLTFGGGTKVEIKRSDAQPSVFLFQPSLDELHTGSASIVCILNDFYPKEVNVKWKV
DGVVQNKGIQESTTEQNSKDSTYSLSSTLTMSSTEYQSHEKFSCEVTHKSLASTLVKSFNRSECQRE

### Inhibition of Feline NGF Bioactivity in TF-1 Cells: TF-1 Cell-Based Assay to Test Blocking Activity of Felinized Antibodies

### Materials

TF1 cell line (CRL-2003)
Growth medium: RPMI-1640 (ThermoFisher CAT #11875-085), 10% FBS and 2 ng/mL rhGM-CSF (R&D, 7954-GM)
Assay Medium: RPMI-1640 (ThermoFisher CAT #11875-085) with 10% FBS CellTiter-Glo® One Solution Assay (Promega cat #G8461)

### Methods

TF-1 is a human erythroleukemic cell line that expresses human TrkA receptor and proliferates in response to NGF from various species. The effect of feline NGF on proliferation of TF-1 cells and the ability of chimeric and felinized anti-NGF antibodies to block proliferation of TF-1 cells were assessed as follows:

### TF-1 Cell Proliferation Mediated by Feline Beta-NGF Assay:

1. Cells are incubated in a T75 flask in a cell culture incubator at 37° C. with 5% CO₂ and greater than 80% RH. The cells are passaged every 3-4 days, if cells seeded as 4-8×10⁴ cells/mL in growth medium.
2. Passage the cells one day before cell proliferation assay conducted.
3. In duplicate, 3-fold dilute feline NGF (fNGF) by adding 75 μL of 200 nM fNGF into the first well of a 96-well assay plate, transferring 25 μL to the next well containing 50 μL assay medium of each column or row all the way down to the last well, and discarding the final 25 μL from the last well.
4. Harvest the TF-1 cells and wash 3× in assay medium. Resuspend the cells in assay medium to a concentration of 0.5-1×10⁶ cells/mL. Transfer 50 μL of the TF-1 cells to each well of above assay plate
5. Incubate the plate for 48-72 hours in a cell culture incubator at 37° C. with 5% CO₂ and greater than 80% RH.
6. Add 30 μL/well of CellTiter®-Glo One Solution Assay into the plate and mix by gentle pipette up-down a couple times. Incubate at room temperature for 10-15 minutes.
7. Measure luminescence intensity with a plate reader.
   Inhibition of fNGF Mediated TF-1 Cell Proliferation by Anti-fNGF Antibodies
1. Cells are incubated in a T75 flask in a cell culture incubator at 37° C. with 5% CO₂ and greater than 80% RH. The cells are passaged every 3-4 days, if cells seeded as 4-8×10⁴ cells/mL in growth medium.
2. Passage the cells one day before cell proliferation assay conducted.
3. In duplicate, 3-fold dilute antibody by adding 75 μL of 240 nM antibody into the first well of a 96-well dilution plate, then transferring 25 μL to next well containing 50 μL assay medium of each column or row all the way down to the last well and discarding the final 25 μL from the last well. For the mAb iso-control, wells with assay medium and cell only are included.
4. Add 10 μL of 12 nM FNGF into each well, and mix by gentle shaking for 2 minutes.
5. Harvest the TF-1 cells and wash 3× in assay medium. Resuspend the cells in assay medium to a concentration of 0.5-1×10⁶ cells/mL.
6. Add 50 μL of the TF-1 cells to each well of an assay 96-well plate, then transfer 50 μL of the mixed fFNGF/antibody from the dilution plate of step 8 to each well of the plate.
7. Incubate the plate for 48-72 hours in a cell culture incubator at 37° C. with 5% CO₂ and greater than 80% RH.
8. Add 30 μL/well of CellTiter*-Glo One Solution Assay into the plate. Mix the contents for 2 minutes on an orbital shaker and incubate at room temperature for 10-15 minutes.
9. Measure luminescence intensity with a plate reader.

### Results

Eight felinized variants were designed and expressed in HEK293 cells (see, Table 7 below). Four out of the eight (including fFulVH2-flgGlam4/fFulVL1-fCk, fFulVH2-flgGlam4/fFulVL2-fCk, fFulVH4-flgGlam4/fFulVL1-fCk, and fFulVH4-flgGlam4/fFulVL2-fCk) were successfully expressed. The inexpressibility of the other four may represent the variants that cannot form functional antibodies.

TABLE 7
Expression of Felinized Fulranumab in HEK 293 cells

| | | Purity | Purity |
|---|---|---|---|
| | Con- | SDS- | SEC- |
| | centration | PAGE | HPLC |
| Antibody ID | (mg/ml) | (%) | (%) |
| fFulVH1-fIgG1am4/fFulVL1-fCk | 0.01 | ≥95 | 92 |
| fFulVH2-fIgG1am4/fFulVL1-fCk | 1.09 | ≥90 | 83 |
| fFulVH3-fIgG1am4/fFulVL1-fCk | 0.01 | ≥95 | 92 |
| fFulVH4-fIgG1am4/fFulVL1-fCk | 0.19 | ≥95 | 98 |
| fFulVH1-fIgG1am4/fFulVL2-fCk | 0.1 | ≥95 | 97 |
| fFulVH2-fIgG1am4/fFulVL2-fCk | 0.89 | ≥90 | 83 |
| fFulVH3-fIgG1am4/fFulVL2-fCk | 0.01 | ≥95 | N/A |
| fFulVH4-fIgG1am4/fFulVL2-fCk | 0.44 | ≥95 | 89 |

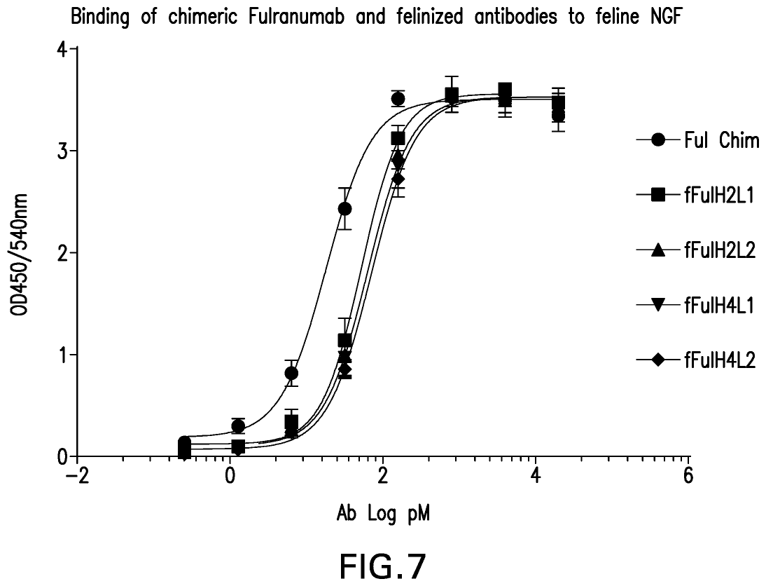

FIG. 7 depicts the binding of the human-feline chimeric Fulranumab (Ful Chim), and the listed felinized variants of Fulranumab as determined by ELISA. As shown in FIG. 7, the chimeric Fulranumab binds to feline NGF with an EC50 of 18-19 pM, whereas the felinized variants of Fulranumab bind to feline NGF with a range of EC50 from 50-70 pM. These results indicate that the felinized antibodies have strong binding affinity for feline NGF and make them suitable for development as drugs for treatment of pain in cats.

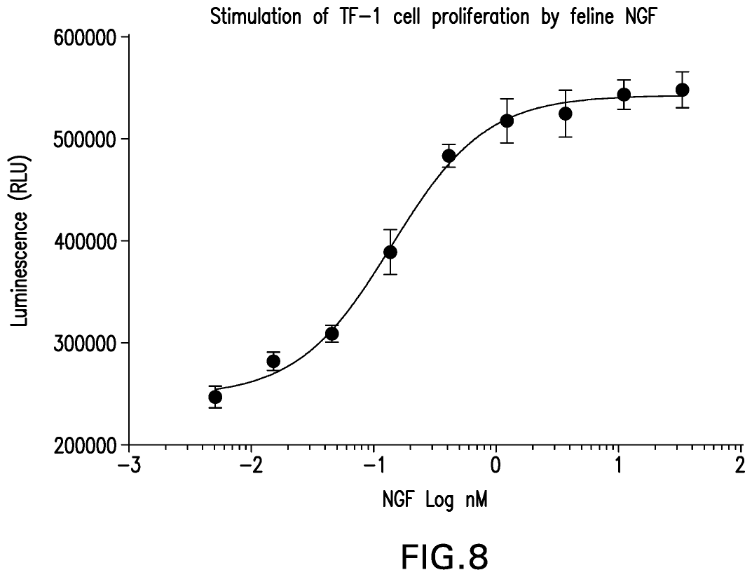

The C-terminal portion of feline NGF was expressed and purified from CHO cells. The ability of feline NGF to stimulate proliferation of TF-1 cells was determined by a bioassay in order to develop an assay to measure the ability of felinized anti-feline NGF antibodies to block downstream signaling and inhibit cell proliferation induced by the binding of feline NGF to the TrkA receptor on TF-1 cells. As shown in FIG. 8, the feline NGF binds with a strong affinity (EC50 0.14 nM) and in a dose dependent manner to TF-1 cells (via binding to endogenous TrkA receptors) and stimulates TF-1 cell proliferation. This result demonstrates that the TF-1 cell-based assay can be used to test the blocking activity of anti-feline NGF antibodies.

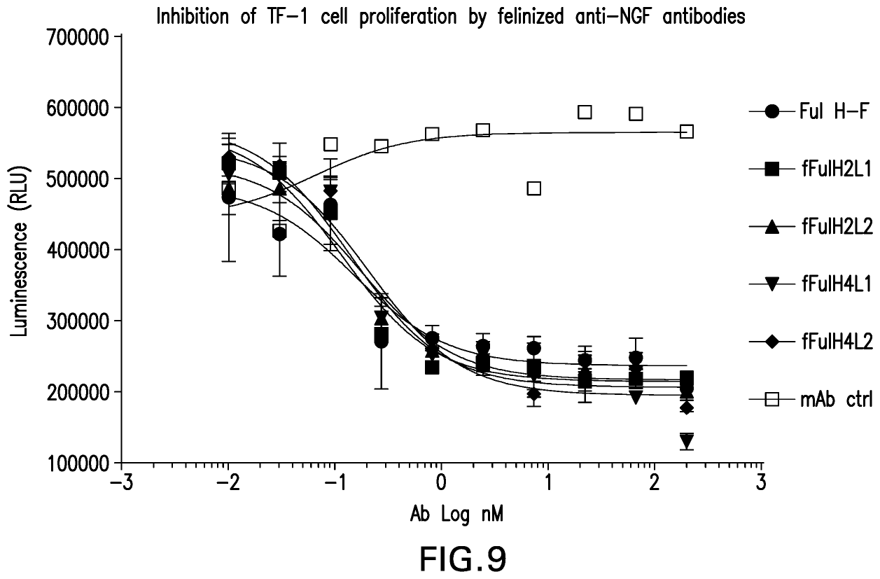

FIG. 9 shows the ability of human-feline chimeric Fulranumab (Ful Chim), the listed felinized variants of Fulranumab, and an isotype control mAb (mAb ctrl) to block TF-1 cell proliferation. The determination was made in a bioassay with TF-1 cells. As shown in FIG. 9, the chimeric and the felinized variants of Fulranumab [fFulVH2-flgGlam4/fFulVL1-fCk, fFulVH4-flgGlam4/fFulVL1-fCk, fFulVH2-flgGlam4/fFulVL2-fCk, and fFulVH4-flgGlam4/fFulVL2-fCk]specifically and in a dose-dependent manner inhibited TF-1 cell proliferations within a range of IC50 from 0.1-0.2 nM. These results indicate that the felinized antibodies are suitable for development as drugs for treatment of pain in cats.

### ST.25 Sequence Listing from an Earlier Filing

```
SEQUENCE LISTING
<110> Intervet Inc.
Intervet International BV
Morsey, Mohamad
Zhang, Yuanzheng
<120> Caninized and Felinized Antibodies to Human NGF
<130> 25370 US-PSP-2
<160> 70
<170> PatentIn version 3.5
<210> 1
<211> 5
<212> PRT
<213> Homo sapiens
<400> 1
Ser Tyr Ser Met Asn
1 5
<210> 2
<211> 17
<212> PRT
<213> Homo sapiens
<400> 2
Tyr Ile Ser Arg Ser Ser His Thr Ile Phe Tyr Ala Asp Ser Val Lys
1 5 10 15
Gly
<210> 3
<211> 14
<212> PRT
<213> Homo sapiens
<400> 3
Val Tyr Ser Ser Gly Trp His Val Ser Asp Tyr Phe Asp Tyr
1 5 10
<210> 4
<211> 1
<212> PRT
<213> Homo sapiens
<400> 4
Arg Ala Ser Gln Gly Ile Ser Ser Ala Leu Ala
1 5 10
<210> 5
<211> 7
<212> PRT
<213> Homo sapiens
<400> 5
Asp Ala Ser Ser Leu Glu Ser
1 5
<210> 6
<211> 9
<212> PRT
<213> Homo sapiens
<400> 6
Gln Gln Phe Asn Ser Tyr Pro Leu Thr
1 5
<210> 7
<211> 5
<212> PRT
<213> Homo sapiens
<400> 7
Glu Leu Ser Ile His
1 5
<210> 8
<211> 1
<212> PRT
<213> Homo sapiens
<400> 8
Gly Phe Asp Pro Glu Asp Gly Glu Thr Ile Tyr Ala Gln Lys Phe Gln
1 5 10 15
Gly
<210> 9
<211> 9
<212> PRT
<213> Homo sapiens
<400> 9
Ile Gly Val Val Thr Asn Phe Asp Asn
1 5
<210> 10
<211> 11
<212> PRT
<213> Homo sapiens
<400> 10
Arg Ala Ser Gln Ala Ile Arg Asn Asp Leu Gly
1 5 10
<210> 11
<211> 7
<212> PRT
<213> Homo sapiens
<400> 11
Ala Ala Phe Asn Leu Gln Ser
1 5
<210> 12
<211> 9
<212> PRT
<213> Homo sapiens
<400> 12
Gln Gln Tyr Asn Arg Tyr Pro Trp Thr
1 5
<210> 13
<211> 223
<212> PRT
<213> Canis familiaris
<400> 13
Glu Pro His Pro Glu Ser His Val Pro Ala Gly His Ala Ile Pro His
1 5 10 15
Ala His Trp Thr Lys Leu Gln His Ser Leu Asp Thr Ala Leu Arg Arg
                20 25 30
Ala Arg Ser Ala Pro Ala Gly Ala Ile Ala Ala Arg Val Thr Gly Gln
          35 40 45
Thr Arg Asn Ile Thr Val Asp Pro Lys Leu Phe Lys Lys Arg Arg Leu
    50 55 60
Arg Ser Pro Arg Val Leu Phe Ser Thr His Pro Pro Pro Val Ala Ala
65 70 75 80
Asp Ala Gln Asp Leu Asp Leu Glu Ala Gly Ser Thr Ala Ser Val Asn
                  85 90 95
Arg Thr His Arg Ser Lys Arg Ser Ser Ser His Pro Val Phe His Arg
            100 105 110
Gly Glu Phe Ser Val Cys Asp Ser Val Ser Val Trp Val Gly Asp Lys
          115 120 125
Thr Thr Ala Thr Asp Ile Lys Gly Lys Glu Val Met Val Leu Gly Glu
    130 135 140
Val Asn Ile Asn Asn Ser Val Phe Lys Gln Tyr Phe Phe Glu Thr Lys
145 150 155 160
Cys Arg Asp Pro Thr Pro Val Asp Ser Gly Cys Arg Gly Ile Asp Ser
                  165 170 175
Lys His Trp Asn Ser Tyr Cys Thr Thr Thr His Thr Phe Val Lys Ala
              180 185 190
Leu Thr Met Asp Gly Lys Gln Ala Ala Trp Arg Phe Ile Arg Ile Asp
          195 200 205
Thr Ala Cys Val Cys Val Leu Ser Arg Lys Ala Gly Arg Arg Ala
    210 215 220
<210> 14
<211> 244
<212> PRT
<213> Artificial Sequence
<220>
<223> modified canine
<400> 14
Glu Pro His Pro Glu Ser His Val Pro Ala Gly His Ala Ile Pro His
1 5 10 15
Ala His Trp Thr Lys Leu Gln His Ser Leu Asp Thr Ala Leu Arg Arg
                20 25 30
Ala Arg Ser Ala Pro Ala Gly Ala Ile Ala Ala Arg Val Thr Gly Gln
          35 40 45
Thr Arg Asn Ile Thr Val Asp Pro Lys Leu Phe Lys Lys Arg Arg Leu
    50 55 60
Arg Ser Pro Arg Val Leu Phe Ser Thr His Pro Pro Pro Val Ala Ala
65 70 75 80
Asp Ala Gln Asp Leu Asp Leu Glu Ala Gly Ser Thr Ala Ser Val Asn
                  85 90 95
Arg Thr His Arg Ser Lys Arg Ser Ser Ser His Pro Val Phe His Arg
            100 105 110
Gly Glu Phe Ser Val Cys Asp Ser Val Ser Val Trp Val Gly Asp Lys
          115 120 125
Thr Thr Ala Thr Asp Ile Lys Gly Lys Glu Val Met Val Leu Gly Glu
    130 135 140
Val Asn Ile Asn Asn Ser Val Phe Lys Gln Tyr Phe Phe Glu Thr Lys
145 150 155 160
Cys Arg Asp Pro Thr Pro Val Asp Ser Gly Cys Arg Gly Ile Asp Ser
                  165 170 175
Lys His Trp Asn Ser Tyr Cys Thr Thr Thr His Thr Phe Val Lys Ala
              180 185 190
Leu Thr Met Asp Gly Lys Gln Ala Ala Trp Arg Phe Ile Arg Ile Asp
          195 200 205
Thr Ala Cys Val Cys Val Leu Ser Arg Lys Ala Gly Arg Arg Ala His
    210 215 220
His His His His His Gly Leu Asn Asp Ile Phe Glu Ala Gln Lys Ile
225 230 235 240
Glu Trp His Glu
<210> 15
<211> 796
<212> PRT
<213> Canis familiaris
<400> 15
Met Leu Arg Gly Gly Arg Leu Gly Gln Arg Gly Gly His Gly Arg Ala
1 5 10 15
Ala Gly Pro Gly Ser Leu Leu Ala Trp Leu Val Leu Ala Ser Ala Gly
                20 25 30
Ala Ala Pro Cys Pro Asp Val Cys Cys Pro His Gly Pro Ser Gly Leu
          35 40 45
Arg Cys Thr Arg Ala Gly Ala Leu Gln Ser Leu His Arg Leu Pro Gly
    50 55 60
Val Glu Asn Leu Thr Glu Leu Tyr Ile Asp Asn Gln Glu His Leu Gln
65 70 75 80
His Leu Asp Ala Val His Leu Lys Gly Leu Gly Met Leu Arg Asp Leu
                  85 90 95
Thr Ile Val Lys Ser Gly Leu Arg Ser Val Ala Pro Asp Ala Phe His
            100 105 110
Phe Thr Pro Arg Leu Arg Arg Leu Asn Leu Ser Phe Asn Ala Leu Glu
          115 120 125
Ser Leu Ser Trp Lys Thr Val Gln Gly Leu Pro Leu Gln Glu Leu Val
    130 135 140
Leu Ser Gly Asn Pro Leu His Cys Ser Cys Ala Leu His Trp Leu Leu
145 150 155 160
Arg Trp Glu Glu Glu Gly Leu Gly Gly Val Arg Gly Gln Arg Leu Gln
                  165 170 175
Cys Pro Gly Gln Gly Pro Leu Ala Leu Leu Ser Asn Ala Ser Cys Gly
              180 185 190
Val Pro Val Leu Lys Val Gln Met Pro Asn Ala Ser Val Glu Val Gly
          195 200 205
Asp Asp Val Leu Leu Gln Cys Gln Val Glu Gly Gln Gly Leu Glu Arg
    210 215 220
Ala Gly Trp Ile Leu Pro Glu Val Glu Glu Leu Ala Thr Val Thr Gln
225 230 235 240
Ser Gly Asp Leu Pro Ser Leu Gly Leu Thr Leu Ala Asn Val Thr Ser
                    245 250 255
Asp Leu Asn Arg Lys Asn Val Thr Cys Trp Ala Glu Asn Asp Val Gly
            260 265 270
Arg Ala Glu Val Ser Val Gln Val Asn Val Ser Phe Pro Ala Ser Val
        275 280 285
Gln Leu His Glu Ala Val Glu Leu His His Trp Cys Ile Pro Phe Ser
  290 295 300
Val Asp Gly Gln Pro Ala Pro Ser Leu Arg Trp Leu Phe Asn Gly Ser
305 310 315 320
Val Leu Asn Glu Thr Ser Phe Ile Phe Thr Glu Phe Leu Glu Pro Val
                    325 330 335
Ala Asn Glu Thr Val Arg His Gly Cys Leu Arg Leu Asn Gln Pro Thr
              340 345 350
His Val Asn Asn Gly Asn Tyr Thr Leu Leu Ala Ala Asn Pro Ser Gly
          355 360 365
Arg Ala Ala Ala Phe Val Met Ala Ala Phe Met Asp Asn Pro Phe Glu
    370 375 380
Phe Asn Pro Glu Asp Pro Ile Pro Val Ser Phe Ser Pro Val Asp Thr
385 390 395 400
Asn Ser Thr Ser Gly Asp Pro Val Glu Lys Lys Asp Glu Thr Pro Phe
                    405 410 415
Gly Val Ser Val Ala Val Gly Leu Ala Val Phe Ala Cys Leu Phe Leu
              420 425 430
Ser Thr Leu Phe Leu Ala Leu Asn Lys Cys Gly Arg Arg Asn Lys Phe
          435 440 445
Gly Gly Asn Arg Ala Val Val Leu Ala Pro Glu Asp Gly Leu Ala Met
    450 455 460
Ser Leu His Phe Met Thr Leu Gly Gly Ser Ser Leu Ser Pro Thr Glu
465 470 475 480
Gly Lys Gly Ser Gly Leu Gln Gly His Ile Ile Glu Asn Pro Gln Tyr
                  485 490 495
Phe Ser Asp Ala Cys Val His His Ile Lys Arg Gln Asp Ile Val Leu
            500 505 510
Lys Trp Glu Leu Gly Glu Gly Ala Phe Gly Lys Val Phe Leu Ala Glu
        515 520 525
Cys His Asn Leu Leu Pro Glu Gln Asp Lys Met Leu Val Ala Val Lys
    530 535 540
Ala Leu Lys Glu Val Ser Glu Ser Ala Arg Gln Asp Phe Gln Arg Glu
545 550 555 560
Ala Gln Leu Leu Thr Met Leu Gln His Gln His Ile Val Arg Phe Phe
                    565 570 575
Gly Val Cys Thr Glu Gly Arg Pro Leu Leu Met Val Phe Glu Tyr Met
              580 585 590
Arg His Gly Asp Leu Asn Arg Phe Leu Arg Ser His Gly Pro Asp Ala
      595 600 605
Lys Leu Leu Ala Gly Gly Glu Asp Val Ala Pro Gly Pro Leu Gly Leu
    610 615 620
Gly Gln Leu Leu Ala Val Ala Ser Gln Val Ala Ala Gly Met Val Tyr
625 630 635 640
Leu Ala Gly Leu His Phe Val His Arg Asp Leu Ala Thr Arg Asn Cys
                    645 650 655
Leu Val Gly Gln Gly Leu Val Val Lys Ile Gly Asp Phe Gly Met Ser
              660 665 670
Arg Asp Ile Tyr Ser Thr Asp Tyr Tyr Arg Val Gly Gly Arg Thr Met
      675 680 685
Leu Pro Ile Arg Trp Met Pro Pro Glu Ser Ile Leu Tyr Arg Lys Phe
    690 695 700
Thr Thr Glu Ser Asp Val Trp Ser Phe Gly Val Val Leu Trp Glu Ile
705 710 715 720
Phe Thr Tyr Gly Lys Gln Pro Trp Tyr Gln Leu Ser Asn Thr Glu Ala
                    725 730 735
Ile Glu Cys Ile Thr Gln Gly Arg Glu Leu Glu Arg Pro Arg Ala Cys
                740 745 750
Pro Pro Glu Val Tyr Ala Ile Met Arg Gly Cys Trp Gln Arg Glu Pro
        755 760 765
Gln Gln Arg His Ser Ile Lys Asp Val His Ala Arg Leu Gln Ala Leu
    770 775 780
Ala Gln Ala Pro Pro Val Tyr Leu Asp Val Leu Gly
785 790 795
<210> 16
<211> 455
<212> PRT
<213> Artificial Sequence
<220>
<223> canine-human fusion protein
<400> 16
Glu Pro His Pro Glu Ser His Val Pro Ala Gly His Ala Ile Pro His
1 5 10 15
Ala His Trp Thr Lys Leu Gln His Ser Leu Asp Thr Ala Leu Arg Arg
                20 25 30
Ala Arg Ser Ala Pro Ala Gly Ala Ile Ala Ala Arg Val Thr Gly Gln
          35 40 45
Thr Arg Asn Ile Thr Val Asp Pro Lys Leu Phe Lys Lys Arg Arg Leu
    50 55 60
Arg Ser Pro Arg Val Leu Phe Ser Thr His Pro Pro Pro Val Ala Ala
65 70 75 80
Asp Ala Gln Asp Leu Asp Leu Glu Ala Gly Ser Thr Ala Ser Val Asn
                  85 90 95
Arg Thr His Arg Ser Lys Arg Ser Ser Ser His Pro Val Phe His Arg
            100 105 110
Gly Glu Phe Ser Val Cys Asp Ser Val Ser Val Trp Val Gly Asp Lys
          115 120 125
Thr Thr Ala Thr Asp Ile Lys Gly Lys Glu Val Met Val Leu Gly Glu
    130 135 140
Val Asn Ile Asn Asn Ser Val Phe Lys Gln Tyr Phe Phe Glu Thr Lys
145 150 155 160
Cys Arg Asp Pro Thr Pro Val Asp Ser Gly Cys Arg Gly Ile Asp Ser
                  165 170 175
Lys His Trp Asn Ser Tyr Cys Thr Thr Thr His Thr Phe Val Lys Ala
              180 185 190
Leu Thr Met Asp Gly Lys Gln Ala Ala Trp Arg Phe Ile Arg Ile Asp
          195 200 205
Thr Ala Cys Val Cys Val Leu Ser Arg Lys Ala Gly Arg Arg Ala Glu
    210 215 220
Pro Lys Ser Cys Asp Lys Thr His Thr Cys Pro Pro Cys Pro Ala Pro
225 230 235 240
Glu Leu Leu Gly Gly Pro Ser Val Phe Leu Phe Pro Pro Lys Pro Lys
                    245 250 255
Asp Thr Leu Met Ile Ser Arg Thr Pro Glu Val Thr Cys Val Val Val
            260 265 270
Asp Val Ser His Glu Asp Pro Glu Val Lys Phe Asn Trp Tyr Val Asp
        275 280 285
Gly Val Glu Val His Asn Ala Lys Thr Lys Pro Arg Glu Glu Gln Tyr
  290 295 300
Asn Ser Thr Tyr Arg Val Val Ser Val Leu Thr Val Leu His Gln Asp
305 310 315 320
Trp Leu Asn Gly Lys Glu Tyr Lys Cys Lys Val Ser Asn Lys Ala Leu
                    325 330 335
Pro Ala Pro Ile Glu Lys Thr Ile Ser Lys Ala Lys Gly Gln Pro Arg
              340 345 350
Glu Pro Gln Val Tyr Thr Leu Pro Pro Ser Arg Asp Glu Leu Thr Lys
          355 360 365
Asn Gln Val Ser Leu Thr Cys Leu Val Lys Gly Phe Tyr Pro Ser Asp
    370 375 380
Ile Ala Val Glu Trp Glu Ser Asn Gly Gln Pro Glu Asn Asn Tyr Lys
385 390 395 400
Thr Thr Pro Pro Val Leu Asp Ser Asp Gly Ser Phe Phe Leu Tyr Ser
                    405 410 415
Lys Leu Thr Val Asp Lys Ser Arg Trp Gln Gln Gly Asn Val Phe Ser
              420 425 430
Cys Ser Val Met His Glu Ala Leu His Asn His Tyr Thr Gln Lys Ser
          435 440 445
Leu Ser Leu Ser Pro Gly Lys
    450 455
<210> 17
<211> 629
<212> PRT
<213> Canis familiaris
<400> 17
Ala Ala Pro Cys Pro Asp Val Cys Cys Pro His Gly Pro Ser Gly Leu
1 5 10 15
Arg Cys Thr Arg Ala Gly Ala Leu Gln Ser Leu His Arg Leu Pro Gly
                20 25 30
Val Glu Asn Leu Thr Glu Leu Tyr Ile Asp Asn Gln Glu His Leu Gln
          35 40 45
His Leu Asp Ala Val His Leu Lys Gly Leu Gly Met Leu Arg Asp Leu
    50 55 60
Thr Ile Val Lys Ser Gly Leu Arg Ser Val Ala Pro Asp Ala Phe His
65 70 75 80
Phe Thr Pro Arg Leu Arg Arg Leu Asn Leu Ser Phe Asn Ala Leu Glu
                  85 90 95
Ser Leu Ser Trp Lys Thr Val Gln Gly Leu Pro Leu Gln Glu Leu Val
            100 105 110
Leu Ser Gly Asn Pro Leu His Cys Ser Cys Ala Leu His Trp Leu Leu
          115 120 125
Arg Trp Glu Glu Glu Gly Leu Gly Gly Val Arg Gly Gln Arg Leu Gln
    130 135 140
Cys Pro Gly Gln Gly Pro Leu Ala Leu Leu Ser Asn Ala Ser Cys Gly
145 150 155 160
Val Pro Val Leu Lys Val Gln Met Pro Asn Ala Ser Val Glu Val Gly
                  165 170 175
Asp Asp Val Leu Leu Gln Cys Gln Val Glu Gly Arg Gly Leu Glu Arg
              180 185 190
Ala Gly Trp Ile Leu Pro Glu Val Glu Glu Leu Ala Thr Val Thr Gln
          195 200 205
Ser Gly Asp Leu Pro Ser Leu Gly Leu Thr Leu Ala Asn Val Thr Ser
    210 215 220
Asp Leu Asn Arg Lys Asn Val Thr Cys Trp Ala Glu Asn Asp Val Gly
225 230 235 240
Arg Ala Glu Val Ser Val Gln Val Asn Val Ser Phe Pro Ala Ser Val
                    245 250 255
Gln Leu His Glu Ala Val Glu Leu His His Trp Cys Ile Pro Phe Ser
            260 265 270
Val Asp Gly Gln Pro Ala Pro Ser Leu Arg Trp Leu Phe Asn Gly Ser
        275 280 285
Val Leu Asn Glu Thr Ser Phe Ile Phe Thr Glu Phe Leu Glu Pro Val
  290 295 300
Ala Asn Glu Thr Val Arg His Gly Cys Leu Arg Leu Asn Gln Pro Thr
305 310 315 320
His Val Asn Asn Gly Asn Tyr Thr Leu Leu Ala Ala Asn Pro Ser Gly
                    325 330 335
Arg Ala Ala Ala Phe Val Met Ala Ala Phe Met Asp Asn Pro Phe Glu
              340 345 350
Phe Asn Pro Glu Asp Pro Ile Pro Val Ser Phe Ser Pro Val Asp Thr
          355 360 365
Asn Ser Thr Ser Gly Asp Pro Val Glu Lys Lys Asp Glu Thr Pro Phe
    370 375 380
Gly Val Ser Val Ala Val Gly Val Pro Lys Arg Glu Asn Gly Arg Val
385 390 395 400
Pro Arg Pro Pro Asp Cys Pro Lys Cys Pro Ala Pro Glu Met Leu Gly
                    405 410 415
Gly Pro Ser Val Phe Ile Phe Pro Pro Lys Pro Lys Asp Thr Leu Leu
              420 425 430
Ile Ala Arg Thr Pro Glu Val Thr Cys Val Val Val Asp Leu Asp Pro
          435 440 445
Glu Asp Pro Glu Val Gln Ile Ser Trp Phe Val Asp Gly Lys Gln Met
    450 455 460
Gln Thr Ala Lys Thr Gln Pro Arg Glu Glu Gln Phe Asn Gly Thr Tyr
465 470 475 480
Arg Val Val Ser Val Leu Pro Ile Gly His Gln Asp Trp Leu Lys Gly
                  485 490 495
Lys Gln Phe Thr Cys Lys Val Asn Asn Lys Ala Leu Pro Ser Pro Ile
            500 505 510
Glu Arg Thr Ile Ser Lys Ala Arg Gly Gln Ala His Gln Pro Ser Val
        515 520 525
Tyr Val Leu Pro Pro Ser Arg Glu Glu Leu Ser Lys Asn Thr Val Ser
    530 535 540
Leu Thr Cys Leu Ile Lys Asp Phe Phe Pro Pro Asp Ile Asp Val Glu
545 550 555 560
Trp Gln Ser Asn Gly Gln Gln Glu Pro Glu Ser Lys Tyr Arg Thr Thr
                    565 570 575
Pro Pro Gln Leu Asp Glu Asp Gly Ser Tyr Phe Leu Tyr Ser Lys Leu
              580 585 590
Ser Val Asp Lys Ser Arg Trp Gln Arg Gly Asp Thr Phe Ile Cys Ala
      595 600 605
Val Met His Glu Ala Leu His Asn His Tyr Thr Gln Glu Ser Leu Ser
    610 615 620
His Ser Pro Gly Lys
625
<210> 18
1
<211> 123
<212> PRT
<213> Homo sapiens
<400> 18
Glu Val Gln Leu Val Glu Ser Gly Gly Gly Leu Val Gln Pro Gly Gly
1 5 10 15
Ser Leu Arg Leu Ser Cys Ala Ala Ser Gly Phe Thr Leu Arg Ser Tyr
                20 25 30
Ser Met Asn Trp Val Arg Gln Ala Pro Gly Lys Gly Leu Glu Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Ser Leu Tyr
65 70 75 80
Leu Gln Met Asp Ser Leu Arg Asp Glu Asp Thr Ala Met Tyr Tyr Cys
                  85 90 95
Ala Arg Val Tyr Ser Ser Gly Trp His Val Ser Asp Tyr Phe Asp Tyr
            100 105 110
Trp Gly Gln Gly Ile Leu Val Thr Val Ser Ser
          115 120
<210> 19
<211> 107
<212> PRT
<213> Homo sapiens
<400> 19
Ala Ile Gln Leu Thr Gln Ser Pro Ser Ser Leu Ser Ala Ser Val Gly
1 5 10 15
Asp Arg Val Thr Ile Thr Cys Arg Ala Ser Gln Gly Ile Ser Ser Ala
                20 25 30
Leu Ala Trp Tyr Gln Gln Lys Pro Gly Lys Ala Pro Lys Leu Leu Ile
          35 40 45
Tyr Asp Ala Ser Ser Leu Glu Ser Gly Val Pro Ser Arg Phe Ser Gly
    50 55 60
Ser Gly Ser Gly Thr Asp Phe Thr Leu Thr Ile Ser Ser Leu Gln Pro
65 70 75 80
Glu Asp Phe Ala Thr Tyr Tyr Cys Gln Gln Phe Asn Ser Tyr Pro Leu
                  85 90 95
Thr Phe Gly Gly Gly Thr Lys Val Glu Ile Lys
            100 105
<210> 20
<211> 215
<212> PRT
<213> Artificial Sequence
<220>
<223> modified canine
<400> 20
Leu Gly Gly Pro Ser Val Phe Ile Phe Pro Pro Lys Pro Lys Asp Thr
1 5 10 15
Leu Leu Ile Ala Arg Thr Pro Glu Val Thr Cys Val Val Val Ala Leu
                20 25 30
Asp Pro Glu Asp Pro Glu Val Gln Ile Ser Trp Phe Val Asp Gly Lys
          35 40 45
Gln Met Gln Thr Ala Lys Thr Gln Pro Arg Glu Glu Gln Phe Ala Gly
    50 55 60
Thr Tyr Arg Val Val Ser Val Leu Pro Ile Gly His Gln Asp Trp Leu
65 70 75 80
Lys Gly Lys Gln Phe Thr Cys Lys Val Asn Asn Lys Ala Leu Pro Ser
                  85 90 95
Pro Ile Glu Arg Thr Ile Ser Lys Ala Arg Gly Gln Ala His Gln Pro
            100 105 110
Ser Val Tyr Val Leu Pro Pro Ser Arg Glu Glu Leu Ser Lys Asn Thr
          115 120 125
Val Ser Leu Thr Cys Leu Ile Lys Asp Phe Phe Pro Pro Asp Ile Asp
    130 135 140
Val Glu Trp Gln Ser Asn Gly Gln Gln Glu Pro Glu Ser Lys Tyr Arg
145 150 155 160
Thr Thr Pro Pro Gln Leu Asp Glu Asp Gly Ser Tyr Phe Leu Tyr Ser
                  165 170 175
Lys Leu Ser Val Asp Lys Ser Arg Trp Gln Arg Gly Asp Thr Phe Ile
              180 185 190
Cys Ala Val Met His Glu Ala Leu His Asn His Tyr Thr Gln Glu Ser
          195 200 205
Leu Ser His Ser Pro Gly Lys
    210 215
<210> 21
<211> 118
<212> PRT
<213> Homo sapiens
<400> 21
Gln Val Gln Leu Val Gln Ser Gly Ala Glu Val Lys Lys Pro Gly Ala
1 5 10 15
Ser Val Lys Val Ser Cys Lys Val Ser Gly Phe Thr Leu Thr Glu Leu
                20 25 30
Ser Ile His Trp Val Arg Gln Ala Pro Gly Lys Gly Leu Glu Trp Met
          35 40 45
Gly Gly Phe Asp Pro Glu Asp Gly Glu Thr Ile Tyr Ala Gln Lys Phe
    50 55 60
Gln Gly Arg Val Thr Met Thr Glu Asp Thr Ser Thr Asp Thr Ala Tyr
65 70 75 80
Met Glu Leu Thr Ser Leu Arg Ser Glu Asp Thr Ala Val Tyr Tyr Cys
                  85 90 95
Ser Thr Ile Gly Val Val Thr Asn Phe Asp Asn Trp Gly Gln Gly Thr
            100 105 110
Leu Val Thr Val Ser Ser
    115
<210> 22
<211> 107
<212> PRT
<213> Homo sapiens
<400> 22
Asp Ile Gln Met Thr Gln Ser Pro Ser Ser Leu Ser Ala Ser Ala Gly
1 5 10 15
Asp Arg Val Thr Ile Thr Cys Arg Ala Ser Gln Ala Ile Arg Asn Asp
                20 25 30
Leu Gly Trp Tyr Gln Gln Lys Pro Gly Lys Ala Pro Lys Arg Leu Ile
          35 40 45
Tyr Ala Ala Phe Asn Leu Gln Ser Gly Val Pro Ser Arg Phe Ser Gly
    50 55 60
Ser Gly Ser Gly Thr Glu Phe Thr Leu Thr Ile Ser Ser Leu Gln Pro
65 70 75 80
Glu Asp Leu Ala Ser Tyr Tyr Cys Gln Gln Tyr Asn Arg Tyr Pro Trp
                  85 90 95
Thr Phe Gly Gln Gly Thr Lys Val Glu Ile Lys
            100 105
<210> 23
<211> 458
<212> PRT
<213> Artificial Sequence
<220>
<223> Human canine fusion protein
<400> 23
Glu Val Gln Leu Val Glu Ser Gly Gly Gly Leu Val Gln Pro Gly Gly
1 5 10 15
Ser Leu Arg Leu Ser Cys Ala Ala Ser Gly Phe Thr Leu Arg Ser Tyr
                20 25 30
Ser Met Asn Trp Val Arg Gln Ala Pro Gly Lys Gly Leu Glu Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Ser Leu Tyr
65 70 75 80
Leu Gln Met Asp Ser Leu Arg Asp Glu Asp Thr Ala Met Tyr Tyr Cys
                  85 90 95
Ala Arg Val Tyr Ser Ser Gly Trp His Val Ser Asp Tyr Phe Asp Tyr
            100 105 110
Trp Gly Gln Gly Ile Leu Val Thr Val Ser Ser Ala Ser Thr Thr Ala
          115 120 125
Pro Ser Val Phe Pro Leu Ala Pro Ser Cys Gly Ser Thr Ser Gly Ser
    130 135 140
Thr Val Ala Leu Ala Cys Leu Val Ser Gly Tyr Phe Pro Glu Pro Val
145 150 155 160
Thr Val Ser Trp Asn Ser Gly Ser Leu Thr Ser Gly Val His Thr Phe
                  165 170 175
Pro Ser Val Leu Gln Ser Ser Gly Leu Tyr Ser Leu Ser Ser Met Val
              180 185 190
Thr Val Pro Ser Ser Arg Trp Pro Ser Glu Thr Phe Thr Cys Asn Val
          195 200 205
Ala His Pro Ala Ser Lys Thr Lys Val Asp Lys Pro Val Pro Lys Arg
    210 215 220
Glu Asn Gly Arg Val Pro Arg Pro Pro Asp Cys Pro Lys Cys Pro Ala
225 230 235 240
Pro Glu Met Leu Gly Gly Pro Ser Val Phe Ile Phe Pro Pro Lys Pro
                    245 250 255
Lys Asp Thr Leu Leu Ile Ala Arg Thr Pro Glu Val Thr Cys Val Val
            260 265 270
Val Asp Leu Asp Pro Glu Asp Pro Glu Val Gln Ile Ser Trp Phe Val
        275 280 285
Asp Gly Lys Gln Met Gln Thr Ala Lys Thr Gln Pro Arg Glu Glu Gln
  290 295 300
Phe Asn Gly Thr Tyr Arg Val Val Ser Val Leu Pro Ile Gly His Gln
305 310 315 320
Asp Trp Leu Lys Gly Lys Gln Phe Thr Cys Lys Val Asn Asn Lys Ala
                    325 330 335
Leu Pro Ser Pro Ile Glu Arg Thr Ile Ser Lys Ala Arg Gly Gln Ala
              340 345 350
His Gln Pro Ser Val Tyr Val Leu Pro Pro Ser Arg Glu Glu Leu Ser
          355 360 365
Lys Asn Thr Val Ser Leu Thr Cys Leu Ile Lys Asp Phe Phe Pro Pro
    370 375 380
Asp Ile Asp Val Glu Trp Gln Ser Asn Gly Gln Gln Glu Pro Glu Ser
385 390 395 400
Lys Tyr Arg Thr Thr Pro Pro Gln Leu Asp Glu Asp Gly Ser Tyr Phe
                    405 410 415
Leu Tyr Ser Lys Leu Ser Val Asp Lys Ser Arg Trp Gln Arg Gly Asp
              420 425 430
Thr Phe Ile Cys Ala Val Met His Glu Ala Leu His Asn His Tyr Thr
          435 440 445
Gln Glu Ser Leu Ser His Ser Pro Gly Lys
    450 455
<210> 24
<211> 217
<212> PRT
<213> Artificial Sequence
<220>
<223> Human canine fusion protein
<400> 24
Ala Ile Gln Leu Thr Gln Ser Pro Ser Ser Leu Ser Ala Ser Val Gly
1 5 10 15
Asp Arg Val Thr Ile Thr Cys Arg Ala Ser Gln Gly Ile Ser Ser Ala
                20 25 30
Leu Ala Trp Tyr Gln Gln Lys Pro Gly Lys Ala Pro Lys Leu Leu Ile
          35 40 45
Tyr Asp Ala Ser Ser Leu Glu Ser Gly Val Pro Ser Arg Phe Ser Gly
    50 55 60
Ser Gly Ser Gly Thr Asp Phe Thr Leu Thr Ile Ser Ser Leu Gln Pro
65 70 75 80
Glu Asp Phe Ala Thr Tyr Tyr Cys Gln Gln Phe Asn Ser Tyr Pro Leu
                  85 90 95
Thr Phe Gly Gly Gly Thr Lys Val Glu Ile Lys Arg Asn Asp Ala Gln
            100 105 110
Pro Ala Val Tyr Leu Phe Gln Pro Ser Pro Asp Gln Leu His Thr Gly
          115 120 125
Ser Ala Ser Val Val Cys Leu Leu Asn Ser Phe Tyr Pro Lys Asp Ile
    130 135 140
Asn Val Lys Trp Lys Val Asp Gly Val Ile Gln Asp Thr Gly Ile Gln
145 150 155 160
Glu Ser Val Thr Glu Gln Asp Lys Asp Ser Thr Tyr Ser Leu Ser Ser
                  165 170 175
Thr Leu Thr Met Ser Ser Thr Glu Tyr Leu Ser His Glu Leu Tyr Ser
              180 185 190
Cys Glu Ile Thr His Lys Ser Leu Pro Ser Thr Leu Ile Lys Ser Phe
          195 200 205
Gln Arg Ser Glu Cys Gln Arg Val Asp
    210 215
<210> 25
<211> 453
<212> PRT
<213> Artificial Sequence
<220>
<223> Human canine fusion protein
<400> 25
Gln Val Gln Leu Val Gln Ser Gly Ala Glu Val Lys Lys Pro Gly Ala
1 5 10 15
Ser Val Lys Val Ser Cys Lys Val Ser Gly Phe Thr Leu Thr Glu Leu
                20 25 30
Ser Ile His Trp Val Arg Gln Ala Pro Gly Lys Gly Leu Glu Trp Met
          35 40 45
Gly Gly Phe Asp Pro Glu Asp Gly Glu Thr Ile Tyr Ala Gln Lys Phe
    50 55 60
Gln Gly Arg Val Thr Met Thr Glu Asp Thr Ser Thr Asp Thr Ala Tyr
65 70 75 80
Met Glu Leu Thr Ser Leu Arg Ser Glu Asp Thr Ala Val Tyr Tyr Cys
                  85 90 95
Ser Thr Ile Gly Val Val Thr Asn Phe Asp Asn Trp Gly Gln Gly Thr
            100 105 110
Leu Val Thr Val Ser Ser Ala Ser Thr Thr Ala Pro Ser Val Phe Pro
          115 120 125
Leu Ala Pro Ser Cys Gly Ser Thr Ser Gly Ser Thr Val Ala Leu Ala
    130 135 140
Cys Leu Val Ser Gly Tyr Phe Pro Glu Pro Val Thr Val Ser Trp Asn
145 150 155 160
Ser Gly Ser Leu Thr Ser Gly Val His Thr Phe Pro Ser Val Leu Gln
                  165 170 175
Ser Ser Gly Leu Tyr Ser Leu Ser Ser Met Val Thr Val Pro Ser Ser
              180 185 190
Arg Trp Pro Ser Glu Thr Phe Thr Cys Asn Val Ala His Pro Ala Ser
          195 200 205
Lys Thr Lys Val Asp Lys Pro Val Pro Lys Arg Glu Asn Gly Arg Val
    210 215 220
Pro Arg Pro Pro Asp Cys Pro Lys Cys Pro Ala Pro Glu Met Leu Gly
225 230 235 240
Gly Pro Ser Val Phe Ile Phe Pro Pro Lys Pro Lys Asp Thr Leu Leu
                    245 250 255
Ile Ala Arg Thr Pro Glu Val Thr Cys Val Val Val Asp Leu Asp Pro
            260 265 270
Glu Asp Pro Glu Val Gln Ile Ser Trp Phe Val Asp Gly Lys Gln Met
        275 280 285
Gln Thr Ala Lys Thr Gln Pro Arg Glu Glu Gln Phe Asn Gly Thr Tyr
  290 295 300
Arg Val Val Ser Val Leu Pro Ile Gly His Gln Asp Trp Leu Lys Gly
305 310 315 320
Lys Gln Phe Thr Cys Lys Val Asn Asn Lys Ala Leu Pro Ser Pro Ile
                    325 330 335
Glu Arg Thr Ile Ser Lys Ala Arg Gly Gln Ala His Gln Pro Ser Val
              340 345 350
Tyr Val Leu Pro Pro Ser Arg Glu Glu Leu Ser Lys Asn Thr Val Ser
          355 360 365
Leu Thr Cys Leu Ile Lys Asp Phe Phe Pro Pro Asp Ile Asp Val Glu
    370 375 380
Trp Gln Ser Asn Gly Gln Gln Glu Pro Glu Ser Lys Tyr Arg Thr Thr
385 390 395 400
Pro Pro Gln Leu Asp Glu Asp Gly Ser Tyr Phe Leu Tyr Ser Lys Leu
                    405 410 415
Ser Val Asp Lys Ser Arg Trp Gln Arg Gly Asp Thr Phe Ile Cys Ala
              420 425 430
Val Met His Glu Ala Leu His Asn His Tyr Thr Gln Glu Ser Leu Ser
          435 440 445
His Ser Pro Gly Lys
    450
<210> 26
<211> 217
<212> PRT
<213> Artificial Sequence
<220>
<223> Human canine fusion protein
<400> 26
Asp Ile Gln Met Thr Gln Ser Pro Ser Ser Leu Ser Ala Ser Ala Gly
1 5 10 15
Asp Arg Val Thr Ile Thr Cys Arg Ala Ser Gln Ala Ile Arg Asn Asp
                20 25 30
Leu Gly Trp Tyr Gln Gln Lys Pro Gly Lys Ala Pro Lys Arg Leu Ile
          35 40 45
Tyr Ala Ala Phe Asn Leu Gln Ser Gly Val Pro Ser Arg Phe Ser Gly
    50 55 60
Ser Gly Ser Gly Thr Glu Phe Thr Leu Thr Ile Ser Ser Leu Gln Pro
65 70 75 80
Glu Asp Leu Ala Ser Tyr Tyr Cys Gln Gln Tyr Asn Arg Tyr Pro Trp
                  85 90 95
Thr Phe Gly Gln Gly Thr Lys Val Glu Ile Lys Arg Asn Asp Ala Gln
            100 105 110
Pro Ala Val Tyr Leu Phe Gln Pro Ser Pro Asp Gln Leu His Thr Gly
          115 120 125
Ser Ala Ser Val Val Cys Leu Leu Asn Ser Phe Tyr Pro Lys Asp Ile
    130 135 140
Asn Val Lys Trp Lys Val Asp Gly Val Ile Gln Asp Thr Gly Ile Gln
145 150 155 160
Glu Ser Val Thr Glu Gln Asp Lys Asp Ser Thr Tyr Ser Leu Ser Ser
                  165 170 175
Thr Leu Thr Met Ser Ser Thr Glu Tyr Leu Ser His Glu Leu Tyr Ser
              180 185 190
Cys Glu Ile Thr His Lys Ser Leu Pro Ser Thr Leu Ile Lys Ser Phe
          195 200 205
Gln Arg Ser Glu Cys Gln Arg Val Asp
    210 215
<210> 27
<211> 123
<212> PRT
<213> Artificial Sequence
<220>
<223> Caninized human
<400> 27
Glu Val Gln Leu Val Glu Ser Gly Gly Asp Leu Val Lys Pro Gly Gly
1 5 10 15
Ser Leu Arg Leu Ser Cys Val Ala Ser Gly Phe Thr Phe Ser Ser Tyr
                20 25 30
Ser Met Asn Trp Ile Arg Gln Ala Pro Gly Lys Gly Leu Gln Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Thr Leu Tyr
65 70 75 80
Leu Gln Met Asn Ser Leu Arg Asp Glu Asp Thr Ala Val Tyr Tyr Cys
                  85 90 95
Ala Arg Val Tyr Ser Ser Gly Trp His Val Ser Asp Tyr Phe Asp Tyr
            100 105 110
Trp Gly Gln Gly Thr Leu Val Thr Val Ser Ser
          115 120
<210> 28
<211> 123
<212> PRT
<213> Artificial Sequence
<220>
<223> Caninized human
<400> 28
Glu Val Gln Leu Val Glu Ser Gly Gly Asp Leu Val Lys Pro Gly Gly
1 5 10 15
Ser Leu Arg Leu Ser Cys Val Ala Ser Gly Phe Thr Leu Arg Ser Tyr
                20 25 30
Ser Met Asn Trp Ile Arg Gln Ala Pro Gly Lys Gly Leu Gln Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Thr Leu Tyr
65 70 75 80
Leu Gln Met Asp Ser Leu Arg Asp Glu Asp Thr Ala Val Tyr Tyr Cys
                  85 90 95
Ala Arg Val Tyr Ser Ser Gly Trp His Val Ser Asp Tyr Phe Asp Tyr
            100 105 110
Trp Gly Gln Gly Ile Leu Val Thr Val Ser Ser
          115 120
<210> 29
<211> 107
<212> PRT
<213> Artificial Sequence
<220>
<223> Caninized human
<400> 29
Glu Ile Val Met Thr Gln Ser Pro Ala Ser Leu Ser Leu Ser Gln Glu
1 5 10 15
Glu Lys Val Thr Ile Thr Cys Arg Ala Ser Gln Gly Ile Ser Ser Ala
                20 25 30
Leu Ala Trp Tyr Gln Gln Lys Pro Gly Gln Ala Pro Lys Leu Leu Ile
          35 40 45
Tyr Asp Ala Ser Ser Leu Glu Ser Gly Val Pro Ser Arg Phe Ser Gly
    50 55 60
Ser Gly Ser Gly Thr Asp Phe Ser Phe Thr Ile Ser Ser Leu Glu Pro
65 70 75 80
Glu Asp Val Ala Val Tyr Tyr Cys Gln Gln Phe Asn Ser Tyr Pro Leu
                  85 90 95
Thr Phe Gly Gln Gly Thr Lys Val Glu Ile Lys
            100 105
<210> 30
<211> 107
<212> PRT
<213> Artificial Sequence
<220>
<223> Caninized human
<400> 30
Glu Ile Gln Leu Thr Gln Ser Pro Ala Ser Leu Ser Leu Ser Gln Glu
1 5 10 15
Glu Lys Val Thr Ile Thr Cys Arg Ala Ser Gln Gly Ile Ser Ser Ala
                20 25 30
Leu Ala Trp Tyr Gln Gln Lys Pro Gly Gln Ala Pro Lys Leu Leu Ile
          35 40 45
Tyr Asp Ala Ser Ser Leu Glu Ser Gly Val Pro Ser Arg Phe Ser Gly
    50 55 60
Ser Gly Ser Gly Thr Asp Phe Ser Leu Thr Ile Ser Ser Leu Glu Pro
65 70 75 80
Glu Asp Phe Ala Val Tyr Tyr Cys Gln Gln Phe Asn Ser Tyr Pro Leu
                  85 90 95
Thr Phe Gly Gly Gly Thr Lys Val Glu Ile Lys
            100 105
<210> 31
<211> 118
<212> PRT
<213> Artificial Sequence
<220>
<223> Caninized human
<400> 31
Glu Val Gln Leu Val Gln Ser Gly Ala Glu Val Lys Lys Pro Gly Ala
1 5 10 15
Ser Val Lys Val Ser Cys Lys Thr Ser Gly Tyr Thr Phe Ile Glu Leu
                20 25 30
Ser Ile His Trp Val Arg Gln Ala Pro Gly Ala Gly Leu Asp Trp Met
          35 40 45
Gly Gly Phe Asp Pro Glu Asp Gly Glu Thr Ile Tyr Ala Gln Lys Phe
    50 55 60
Gln Gly Arg Val Thr Leu Thr Ala Asp Thr Ser Thr Ser Thr Ala Tyr
65 70 75 80
Met Glu Leu Ser Ser Leu Arg Ala Gly Asp Ile Ala Val Tyr Tyr Cys
                  85 90 95
Ala Arg Ile Gly Val Val Thr Asn Phe Asp Asn Trp Gly Gln Gly Thr
            100 105 110
Leu Val Thr Val Ser Ser
    115
<210> 32
<211> 118
<212> PRT
<213> Artificial Sequence
<220>
<223> Caninized human
<400> 32
Glu Val Gln Leu Val Gln Ser Gly Ala Glu Val Lys Lys Pro Gly Ala
1 5 10 15
Ser Val Lys Val Ser Cys Lys Val Ser Gly Tyr Thr Leu Thr Glu Leu
                20 25 30
Ser Ile His Trp Val Arg Gln Ala Pro Gly Lys Gly Leu Asp Trp Met
          35 40 45
Gly Gly Phe Asp Pro Glu Asp Gly Glu Thr Ile Tyr Ala Gln Lys Phe
    50 55 60
Gln Gly Arg Val Thr Leu Thr Glu Asp Thr Ser Thr Asp Thr Ala Tyr
65 70 75 80
Met Glu Leu Ser Ser Leu Arg Ala Gly Asp Ile Ala Val Tyr Tyr Cys
                  85 90 95
Ser Thr Ile Gly Val Val Thr Asn Phe Asp Asn Trp Gly Gln Gly Thr
            100 105 110
Leu Val Thr Val Ser Ser
      115
<210> 33
<211> 107
<212> PRT
<213> Artificial Sequence
<220>
<223> Caninized human
<400> 33
Glu Ile Val Met Thr Gln Ser Pro Ala Ser Leu Ser Leu Ser Gln Glu
1 5 10 15
Glu Lys Val Thr Ile Thr Cys Arg Ala Ser Gln Ala Ile Arg Asn Asp
                20 25 30
Leu Gly Trp Tyr Gln Gln Lys Pro Gly Gln Ala Pro Lys Leu Leu Ile
          35 40 45
Tyr Ala Ala Phe Asn Leu Gln Ser Gly Val Pro Ser Arg Phe Ser Gly
    50 55 60
Ser Gly Ser Gly Thr Asp Phe Ser Phe Thr Ile Ser Ser Leu Glu Pro
65 70 75 80
Glu Asp Val Ala Val Tyr Tyr Cys Gln Gln Tyr Asn Arg Tyr Pro Trp
                  85 90 95
Thr Phe Gly Gln Gly Thr Lys Leu Glu Ile Lys
            100 105
<210> 34
<211> 107
<212> PRT
<213> Artificial Sequence
<220>
<223> Caninized human
<400> 34
Asp Ile Val Met Thr Gln Thr Pro Leu Ser Leu Ser Val Ser Pro Gly
1 5 10 15
Glu Thr Ala Ser Ile Ser Cys Arg Ala Ser Gln Ala Ile Arg Asn Asp
                20 25 30
Leu Gly Trp Phe Arg Gln Lys Pro Gly Gln Ser Pro Gln Arg Leu Ile
          35 40 45
Tyr Ala Ala Phe Asn Leu Gln Ser Gly Val Pro Asp Arg Phe Ser Gly
    50 55 60
Ser Gly Ser Gly Thr Asp Phe Thr Leu Arg Ile Ser Arg Val Glu Ala
65 70 75 80
Asp Asp Thr Gly Val Tyr Tyr Cys Gln Gln Tyr Asn Arg Tyr Pro Trp
                  85 90 95
Thr Phe Gly Gln Gly Thr Lys Leu Glu Ile Lys
            100 105
<210> 35
<211> 107
<212> PRT
<213> Artificial Sequence
<220>
<223> caninized human
<400> 35
Asp Ile Val Met Thr Gln Thr Pro Leu Ser Leu Ser Val Ser Pro Gly
1 5 10 15
Glu Thr Ala Ser Ile Ser Cys Arg Ala Ser Gln Ala Ile Arg Asn Asp
                20 25 30
Leu Gly Trp Phe Arg Gln Lys Pro Gly Lys Ser Pro Lys Arg Leu Ile
          35 40 45
Tyr Ala Ala Phe Asn Leu Gln Ser Gly Val Pro Asp Arg Phe Ser Gly
    50 55 60
Ser Gly Ser Gly Thr Asp Phe Thr Leu Thr Ile Ser Ser Val Glu Ala
65 70 75 80
Asp Asp Thr Gly Val Tyr Tyr Cys Gln Gln Tyr Asn Arg Tyr Pro Trp
                  85 90 95
Thr Phe Gly Gln Gly Thr Lys Leu Glu Ile Lys
            100 105
<210> 36
<211> 458
<212> PRT
<213> Artificial Sequence
<220>
<223> Caninized human
<400> 36
Glu Val Gln Leu Val Glu Ser Gly Gly Asp Leu Val Lys Pro Gly Gly
1 5 10 15
Ser Leu Arg Leu Ser Cys Val Ala Ser Gly Phe Thr Phe Ser Ser Tyr
                20 25 30
Ser Met Asn Trp Ile Arg Gln Ala Pro Gly Lys Gly Leu Gln Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Thr Leu Tyr
65 70 75 80
Leu Gln Met Asn Ser Leu Arg Asp Glu Asp Thr Ala Val Tyr Tyr Cys
                  85 90 95
Ala Arg Val Tyr Ser Ser Gly Trp His Val Ser Asp Tyr Phe Asp Tyr
            100 105 110
Trp Gly Gln Gly Thr Leu Val Thr Val Ser Ser Ala Ser Thr Thr Ala
          115 120 125
Pro Ser Val Phe Pro Leu Ala Pro Ser Cys Gly Ser Thr Ser Gly Ser
    130 135 140
Thr Val Ala Leu Ala Cys Leu Val Ser Gly Tyr Phe Pro Glu Pro Val
145 150 155 160
Thr Val Ser Trp Asn Ser Gly Ser Leu Thr Ser Gly Val His Thr Phe
                  165 170 175
Pro Ser Val Leu Gln Ser Ser Gly Leu Tyr Ser Leu Ser Ser Met Val
              180 185 190
Thr Val Pro Ser Ser Arg Trp Pro Ser Glu Thr Phe Thr Cys Asn Val
          195 200 205
Ala His Pro Ala Ser Lys Thr Lys Val Asp Lys Pro Val Pro Lys Arg
    210 215 220
Glu Asn Gly Arg Val Pro Arg Pro Pro Asp Cys Pro Lys Cys Pro Ala
225 230 235 240
Pro Glu Met Leu Gly Gly Pro Ser Val Phe Ile Phe Pro Pro Lys Pro
                    245 250 255
Lys Asp Thr Leu Leu Ile Ala Arg Thr Pro Glu Val Thr Cys Val Val
            260 265 270
Val Asp Leu Asp Pro Glu Asp Pro Glu Val Gln Ile Ser Trp Phe Val
        275 280 285
Asp Gly Lys Gln Met Gln Thr Ala Lys Thr Gln Pro Arg Glu Glu Gln
  290 295 300
Phe Asn Gly Thr Tyr Arg Val Val Ser Val Leu Pro Ile Gly His Gln
305 310 315 320
Asp Trp Leu Lys Gly Lys Gln Phe Thr Cys Lys Val Asn Asn Lys Ala
                    325 330 335
Leu Pro Ser Pro Ile Glu Arg Thr Ile Ser Lys Ala Arg Gly Gln Ala
              340 345 350
His Gln Pro Ser Val Tyr Val Leu Pro Pro Ser Arg Glu Glu Leu Ser
          355 360 365
Lys Asn Thr Val Ser Leu Thr Cys Leu Ile Lys Asp Phe Phe Pro Pro
    370 375 380
Asp Ile Asp Val Glu Trp Gln Ser Asn Gly Gln Gln Glu Pro Glu Ser
385 390 395 400
Lys Tyr Arg Thr Thr Pro Pro Gln Leu Asp Glu Asp Gly Ser Tyr Phe
                    405 410 415
Leu Tyr Ser Lys Leu Ser Val Asp Lys Ser Arg Trp Gln Arg Gly Asp
              420 425 430
Thr Phe Ile Cys Ala Val Met His Glu Ala Leu His Asn His Tyr Thr
          435 440 445
Gln Glu Ser Leu Ser His Ser Pro Gly Lys
    450 455
<210> 37
<211> 458
<212> PRT
<213> Artificial Sequence
<220>
<223> Caninized human
<400> 37
Glu Val Gln Leu Val Glu Ser Gly Gly Asp Leu Val Lys Pro Gly Gly
1 5 10 15
Ser Leu Arg Leu Ser Cys Val Ala Ser Gly Phe Thr Leu Arg Ser Tyr
                20 25 30
Ser Met Asn Trp Ile Arg Gln Ala Pro Gly Lys Gly Leu Gln Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Thr Leu Tyr
65 70 75 80
Leu Gln Met Asp Ser Leu Arg Asp Glu Asp Thr Ala Val Tyr Tyr Cys
                  85 90 95
Ala Arg Val Tyr Ser Ser Gly Trp His Val Ser Asp Tyr Phe Asp Tyr
            100 105 110
Trp Gly Gln Gly Ile Leu Val Thr Val Ser Ser Ala Ser Thr Thr Ala
          115 120 125
Pro Ser Val Phe Pro Leu Ala Pro Ser Cys Gly Ser Thr Ser Gly Ser
    130 135 140
Thr Val Ala Leu Ala Cys Leu Val Ser Gly Tyr Phe Pro Glu Pro Val
145 150 155 160
Thr Val Ser Trp Asn Ser Gly Ser Leu Thr Ser Gly Val His Thr Phe
                  165 170 175
Pro Ser Val Leu Gln Ser Ser Gly Leu Tyr Ser Leu Ser Ser Met Val
              180 185 190
Thr Val Pro Ser Ser Arg Trp Pro Ser Glu Thr Phe Thr Cys Asn Val
          195 200 205
Ala His Pro Ala Ser Lys Thr Lys Val Asp Lys Pro Val Pro Lys Arg
    210 215 220
Glu Asn Gly Arg Val Pro Arg Pro Pro Asp Cys Pro Lys Cys Pro Ala
225 230 235 240
Pro Glu Met Leu Gly Gly Pro Ser Val Phe Ile Phe Pro Pro Lys Pro
                    245 250 255
Lys Asp Thr Leu Leu Ile Ala Arg Thr Pro Glu Val Thr Cys Val Val
            260 265 270
Val Asp Leu Asp Pro Glu Asp Pro Glu Val Gln Ile Ser Trp Phe Val
        275 280 285
Asp Gly Lys Gln Met Gln Thr Ala Lys Thr Gln Pro Arg Glu Glu Gln
  290 295 300
Phe Asn Gly Thr Tyr Arg Val Val Ser Val Leu Pro Ile Gly His Gln
305 310 315 320
Asp Trp Leu Lys Gly Lys Gln Phe Thr Cys Lys Val Asn Asn Lys Ala
                    325 330 335
Leu Pro Ser Pro Ile Glu Arg Thr Ile Ser Lys Ala Arg Gly Gln Ala
              340 345 350
His Gln Pro Ser Val Tyr Val Leu Pro Pro Ser Arg Glu Glu Leu Ser
          355 360 365
Lys Asn Thr Val Ser Leu Thr Cys Leu Ile Lys Asp Phe Phe Pro Pro
    370 375 380
Asp Ile Asp Val Glu Trp Gln Ser Asn Gly Gln Gln Glu Pro Glu Ser
385 390 395 400
Lys Tyr Arg Thr Thr Pro Pro Gln Leu Asp Glu Asp Gly Ser Tyr Phe
                    405 410 415
Leu Tyr Ser Lys Leu Ser Val Asp Lys Ser Arg Trp Gln Arg Gly Asp
              420 425 430
Thr Phe Ile Cys Ala Val Met His Glu Ala Leu His Asn His Tyr Thr
          435 440 445
Gln Glu Ser Leu Ser His Ser Pro Gly Lys
    450 455
<210> 38
<211> 217
<212> PRT
<213> Artificial Sequence
<220>
<223> Caninized human
<400> 38
Glu Ile Val Met Thr Gln Ser Pro Ala Ser Leu Ser Leu Ser Gln Glu
1 5 10 15
Glu Lys Val Thr Ile Thr Cys Arg Ala Ser Gln Gly Ile Ser Ser Ala
                20 25 30
Leu Ala Trp Tyr Gln Gln Lys Pro Gly Gln Ala Pro Lys Leu Leu Ile
          35 40 45
Tyr Asp Ala Ser Ser Leu Glu Ser Gly Val Pro Ser Arg Phe Ser Gly
    50 55 60
Ser Gly Ser Gly Thr Asp Phe Ser Phe Thr Ile Ser Ser Leu Glu Pro
65 70 75 80
Glu Asp Val Ala Val Tyr Tyr Cys Gln Gln Phe Asn Ser Tyr Pro Leu
                  85 90 95
Thr Phe Gly Gln Gly Thr Lys Val Glu Ile Lys Arg Asn Asp Ala Gln
            100 105 110
Pro Ala Val Tyr Leu Phe Gln Pro Ser Pro Asp Gln Leu His Thr Gly
          115 120 125
Ser Ala Ser Val Val Cys Leu Leu Asn Ser Phe Tyr Pro Lys Asp Ile
    130 135 140
Asn Val Lys Trp Lys Val Asp Gly Val Ile Gln Asp Thr Gly Ile Gln
145 150 155 160
Glu Ser Val Thr Glu Gln Asp Lys Asp Ser Thr Tyr Ser Leu Ser Ser
                  165 170 175
Thr Leu Thr Met Ser Ser Thr Glu Tyr Leu Ser His Glu Leu Tyr Ser
              180 185 190
Cys Glu Ile Thr His Lys Ser Leu Pro Ser Thr Leu Ile Lys Ser Phe
          195 200 205
Gln Arg Ser Glu Cys Gln Arg Val Asp
    210 215
<210> 39
<211> 217
<212> PRT
<213> Artificial Sequence
<220>
<223> Caninized human
<400> 39
Glu Ile Gln Leu Thr Gln Ser Pro Ala Ser Leu Ser Leu Ser Gln Glu
1 5 10 15
Glu Lys Val Thr Ile Thr Cys Arg Ala Ser Gln Gly Ile Ser Ser Ala
                20 25 30
Leu Ala Trp Tyr Gln Gln Lys Pro Gly Gln Ala Pro Lys Leu Leu Ile
          35 40 45
Tyr Asp Ala Ser Ser Leu Glu Ser Gly Val Pro Ser Arg Phe Ser Gly
    50 55 60
Ser Gly Ser Gly Thr Asp Phe Ser Leu Thr Ile Ser Ser Leu Glu Pro
65 70 75 80
Glu Asp Phe Ala Val Tyr Tyr Cys Gln Gln Phe Asn Ser Tyr Pro Leu
                  85 90 95
Thr Phe Gly Gly Gly Thr Lys Val Glu Ile Lys Arg Asn Asp Ala Gln
            100 105 110
Pro Ala Val Tyr Leu Phe Gln Pro Ser Pro Asp Gln Leu His Thr Gly
          115 120 125
Ser Ala Ser Val Val Cys Leu Leu Asn Ser Phe Tyr Pro Lys Asp Ile
    130 135 140
Asn Val Lys Trp Lys Val Asp Gly Val Ile Gln Asp Thr Gly Ile Gln
145 150 155 160
Glu Ser Val Thr Glu Gln Asp Lys Asp Ser Thr Tyr Ser Leu Ser Ser
                  165 170 175
Thr Leu Thr Met Ser Ser Thr Glu Tyr Leu Ser His Glu Leu Tyr Ser
              180 185 190
Cys Glu Ile Thr His Lys Ser Leu Pro Ser Thr Leu Ile Lys Ser Phe
          195 200 205
Gln Arg Ser Glu Cys Gln Arg Val Asp
    210 215
<210> 40
<211> 453
<212> PRT
<213> Artificial Sequence
<220>
<223> Caninized human
<400> 40
Glu Val Gln Leu Val Gln Ser Gly Ala Glu Val Lys Lys Pro Gly Ala
1 5 10 15
Ser Val Lys Val Ser Cys Lys Thr Ser Gly Tyr Thr Phe Ile Glu Leu
                20 25 30
Ser Ile His Trp Val Arg Gln Ala Pro Gly Ala Gly Leu Asp Trp Met
          35 40 45
Gly Gly Phe Asp Pro Glu Asp Gly Glu Thr Ile Tyr Ala Gln Lys Phe
    50 55 60
Gln Gly Arg Val Thr Leu Thr Ala Asp Thr Ser Thr Ser Thr Ala Tyr
65 70 75 80
Met Glu Leu Ser Ser Leu Arg Ala Gly Asp Ile Ala Val Tyr Tyr Cys
                  85 90 95
Ala Arg Ile Gly Val Val Thr Asn Phe Asp Asn Trp Gly Gln Gly Thr
            100 105 110
Leu Val Thr Val Ser Ser Ala Ser Thr Thr Ala Pro Ser Val Phe Pro
          115 120 125
Leu Ala Pro Ser Cys Gly Ser Thr Ser Gly Ser Thr Val Ala Leu Ala
    130 135 140
Cys Leu Val Ser Gly Tyr Phe Pro Glu Pro Val Thr Val Ser Trp Asn
145 150 155 160
Ser Gly Ser Leu Thr Ser Gly Val His Thr Phe Pro Ser Val Leu Gln
                  165 170 175
Ser Ser Gly Leu Tyr Ser Leu Ser Ser Met Val Thr Val Pro Ser Ser
              180 185 190
Arg Trp Pro Ser Glu Thr Phe Thr Cys Asn Val Ala His Pro Ala Ser
          195 200 205
Lys Thr Lys Val Asp Lys Pro Val Pro Lys Arg Glu Asn Gly Arg Val
    210 215 220
Pro Arg Pro Pro Asp Cys Pro Lys Cys Pro Ala Pro Glu Met Leu Gly
225 230 235 240
Gly Pro Ser Val Phe Ile Phe Pro Pro Lys Pro Lys Asp Thr Leu Leu
                    245 250 255
Ile Ala Arg Thr Pro Glu Val Thr Cys Val Val Val Asp Leu Asp Pro
            260 265 270
Glu Asp Pro Glu Val Gln Ile Ser Trp Phe Val Asp Gly Lys Gln Met
        275 280 285
Gln Thr Ala Lys Thr Gln Pro Arg Glu Glu Gln Phe Asn Gly Thr Tyr
  290 295 300
Arg Val Val Ser Val Leu Pro Ile Gly His Gln Asp Trp Leu Lys Gly
305 310 315 320
Lys Gln Phe Thr Cys Lys Val Asn Asn Lys Ala Leu Pro Ser Pro Ile
                    325 330 335
Glu Arg Thr Ile Ser Lys Ala Arg Gly Gln Ala His Gln Pro Ser Val
              340 345 350
Tyr Val Leu Pro Pro Ser Arg Glu Glu Leu Ser Lys Asn Thr Val Ser
          355 360 365
Leu Thr Cys Leu Ile Lys Asp Phe Phe Pro Pro Asp Ile Asp Val Glu
    370 375 380
Trp Gln Ser Asn Gly Gln Gln Glu Pro Glu Ser Lys Tyr Arg Thr Thr
385 390 395 400
Pro Pro Gln Leu Asp Glu Asp Gly Ser Tyr Phe Leu Tyr Ser Lys Leu
                    405 410 415
Ser Val Asp Lys Ser Arg Trp Gln Arg Gly Asp Thr Phe Ile Cys Ala
              420 425 430
Val Met His Glu Ala Leu His Asn His Tyr Thr Gln Glu Ser Leu Ser
          435 440 445
His Ser Pro Gly Lys
    450
<210> 41
<211> 453
<212> PRT
<213> Artificial Sequence
<220>
<223> Caninized human
<400> 4
Glu Val Gln Leu Val Gln Ser Gly Ala Glu Val Lys Lys Pro Gly Ala
1 5 10 15
Ser Val Lys Val Ser Cys Lys Val Ser Gly Tyr Thr Leu Thr Glu Leu
                20 25 30
Ser Ile His Trp Val Arg Gln Ala Pro Gly Lys Gly Leu Asp Trp Met
          35 40 45
Gly Gly Phe Asp Pro Glu Asp Gly Glu Thr Ile Tyr Ala Gln Lys Phe
    50 55 60
Gln Gly Arg Val Thr Leu Thr Glu Asp Thr Ser Thr Asp Thr Ala Tyr
65 70 75 80
Met Glu Leu Ser Ser Leu Arg Ala Gly Asp Ile Ala Val Tyr Tyr Cys
                  85 90 95
Ser Thr Ile Gly Val Val Thr Asn Phe Asp Asn Trp Gly Gln Gly Thr
            100 105 110
Leu Val Thr Val Ser Ser Ala Ser Thr Thr Ala Pro Ser Val Phe Pro
          115 120 125
Leu Ala Pro Ser Cys Gly Ser Thr Ser Gly Ser Thr Val Ala Leu Ala
    130 135 140
Cys Leu Val Ser Gly Tyr Phe Pro Glu Pro Val Thr Val Ser Trp Asn
145 150 155 160
Ser Gly Ser Leu Thr Ser Gly Val His Thr Phe Pro Ser Val Leu Gln
                  165 170 175
Ser Ser Gly Leu Tyr Ser Leu Ser Ser Met Val Thr Val Pro Ser Ser
              180 185 190
Arg Trp Pro Ser Glu Thr Phe Thr Cys Asn Val Ala His Pro Ala Ser
          195 200 205
Lys Thr Lys Val Asp Lys Pro Val Pro Lys Arg Glu Asn Gly Arg Val
    210 215 220
Pro Arg Pro Pro Asp Cys Pro Lys Cys Pro Ala Pro Glu Met Leu Gly
225 230 235 240
Gly Pro Ser Val Phe Ile Phe Pro Pro Lys Pro Lys Asp Thr Leu Leu
                    245 250 255
Ile Ala Arg Thr Pro Glu Val Thr Cys Val Val Val Asp Leu Asp Pro
            260 265 270
Glu Asp Pro Glu Val Gln Ile Ser Trp Phe Val Asp Gly Lys Gln Met
        275 280 285
Gln Thr Ala Lys Thr Gln Pro Arg Glu Glu Gln Phe Asn Gly Thr Tyr
  290 295 300
Arg Val Val Ser Val Leu Pro Ile Gly His Gln Asp Trp Leu Lys Gly
305 310 315 320
Lys Gln Phe Thr Cys Lys Val Asn Asn Lys Ala Leu Pro Ser Pro Ile
                    325 330 335
Glu Arg Thr Ile Ser Lys Ala Arg Gly Gln Ala His Gln Pro Ser Val
              340 345 350
Tyr Val Leu Pro Pro Ser Arg Glu Glu Leu Ser Lys Asn Thr Val Ser
          355 360 365
Leu Thr Cys Leu Ile Lys Asp Phe Phe Pro Pro Asp Ile Asp Val Glu
    370 375 380
Trp Gln Ser Asn Gly Gln Gln Glu Pro Glu Ser Lys Tyr Arg Thr Thr
385 390 395 400
Pro Pro Gln Leu Asp Glu Asp Gly Ser Tyr Phe Leu Tyr Ser Lys Leu
                    405 410 415
Ser Val Asp Lys Ser Arg Trp Gln Arg Gly Asp Thr Phe Ile Cys Ala
              420 425 430
Val Met His Glu Ala Leu His Asn His Tyr Thr Gln Glu Ser Leu Ser
          435 440 445
His Ser Pro Gly Lys
    450
<210> 42
<211> 217
<212> PRT
<213> Artificial Sequence
<220>
<223> Caninized human
<400> 42
Glu Ile Val Met Thr Gln Ser Pro Ala Ser Leu Ser Leu Ser Gln Glu
1 5 10 15
Glu Lys Val Thr Ile Thr Cys Arg Ala Ser Gln Ala Ile Arg Asn Asp
                20 25 30
Leu Gly Trp Tyr Gln Gln Lys Pro Gly Gln Ala Pro Lys Leu Leu Ile
          35 40 45
Tyr Ala Ala Phe Asn Leu Gln Ser Gly Val Pro Ser Arg Phe Ser Gly
    50 55 60
Ser Gly Ser Gly Thr Asp Phe Ser Phe Thr Ile Ser Ser Leu Glu Pro
65 70 75 80
Glu Asp Val Ala Val Tyr Tyr Cys Gln Gln Tyr Asn Arg Tyr Pro Trp
                  85 90 95
Thr Phe Gly Gln Gly Thr Lys Leu Glu Ile Lys Arg Asn Asp Ala Gln
            100 105 110
Pro Ala Val Tyr Leu Phe Gln Pro Ser Pro Asp Gln Leu His Thr Gly
          115 120 125
Ser Ala Ser Val Val Cys Leu Leu Asn Ser Phe Tyr Pro Lys Asp Ile
    130 135 140
Asn Val Lys Trp Lys Val Asp Gly Val Ile Gln Asp Thr Gly Ile Gln
145 150 155 160
Glu Ser Val Thr Glu Gln Asp Lys Asp Ser Thr Tyr Ser Leu Ser Ser
                  165 170 175
Thr Leu Thr Met Ser Ser Thr Glu Tyr Leu Ser His Glu Leu Tyr Ser
              180 185 190
Cys Glu Ile Thr His Lys Ser Leu Pro Ser Thr Leu Ile Lys Ser Phe
          195 200 205
Gln Arg Ser Glu Cys Gln Arg Val Asp
    210 215
<210> 43
<211> 217
<212> PRT
<213> Artificial Sequence
<220>
<223> Caninized human
<400> 43
Asp Ile Val Met Thr Gln Thr Pro Leu Ser Leu Ser Val Ser Pro Gly
1 5 10 15
Glu Thr Ala Ser Ile Ser Cys Arg Ala Ser Gln Ala Ile Arg Asn Asp
                20 25 30
Leu Gly Trp Phe Arg Gln Lys Pro Gly Gln Ser Pro Gln Arg Leu Ile
          35 40 45
Tyr Ala Ala Phe Asn Leu Gln Ser Gly Val Pro Asp Arg Phe Ser Gly
    50 55 60
Ser Gly Ser Gly Thr Asp Phe Thr Leu Arg Ile Ser Arg Val Glu Ala
65 70 75 80
Asp Asp Thr Gly Val Tyr Tyr Cys Gln Gln Tyr Asn Arg Tyr Pro Trp
                  85 90 95
Thr Phe Gly Gln Gly Thr Lys Leu Glu Ile Lys Arg Asn Asp Ala Gln
            100 105 110
Pro Ala Val Tyr Leu Phe Gln Pro Ser Pro Asp Gln Leu His Thr Gly
          115 120 125
Ser Ala Ser Val Val Cys Leu Leu Asn Ser Phe Tyr Pro Lys Asp Ile
    130 135 140
Asn Val Lys Trp Lys Val Asp Gly Val Ile Gln Asp Thr Gly Ile Gln
145 150 155 160
Glu Ser Val Thr Glu Gln Asp Lys Asp Ser Thr Tyr Ser Leu Ser Ser
                  165 170 175
Thr Leu Thr Met Ser Ser Thr Glu Tyr Leu Ser His Glu Leu Tyr Ser
              180 185 190
Cys Glu Ile Thr His Lys Ser Leu Pro Ser Thr Leu Ile Lys Ser Phe
          195 200 205
Gln Arg Ser Glu Cys Gln Arg Val Asp
    210 215
<210> 44
<211> 217
<212> PRT
<213> Artificial Sequence
<220>
<223> Caninized human
<400> 44
Asp Ile Val Met Thr Gln Thr Pro Leu Ser Leu Ser Val Ser Pro Gly
1 5 10 15
Glu Thr Ala Ser Ile Ser Cys Arg Ala Ser Gln Ala Ile Arg Asn Asp
                20 25 30
Leu Gly Trp Phe Arg Gln Lys Pro Gly Lys Ser Pro Lys Arg Leu Ile
          35 40 45
Tyr Ala Ala Phe Asn Leu Gln Ser Gly Val Pro Asp Arg Phe Ser Gly
    50 55 60
Ser Gly Ser Gly Thr Asp Phe Thr Leu Thr Ile Ser Ser Val Glu Ala
65 70 75 80
Asp Asp Thr Gly Val Tyr Tyr Cys Gln Gln Tyr Asn Arg Tyr Pro Trp
                  85 90 95
Thr Phe Gly Gln Gly Thr Lys Leu Glu Ile Lys Arg Asn Asp Ala Gln
            100 105 110
Pro Ala Val Tyr Leu Phe Gln Pro Ser Pro Asp Gln Leu His Thr Gly
          115 120 125
Ser Ala Ser Val Val Cys Leu Leu Asn Ser Phe Tyr Pro Lys Asp Ile
    130 135 140
Asn Val Lys Trp Lys Val Asp Gly Val Ile Gln Asp Thr Gly Ile Gln
145 150 155 160
Glu Ser Val Thr Glu Gln Asp Lys Asp Ser Thr Tyr Ser Leu Ser Ser
                  165 170 175
Thr Leu Thr Met Ser Ser Thr Glu Tyr Leu Ser His Glu Leu Tyr Ser
              180 185 190
Cys Glu Ile Thr His Lys Ser Leu Pro Ser Thr Leu Ile Lys Ser Phe
          195 200 205
Gln Arg Ser Glu Cys Gln Arg Val Asp
    210 215
<210> 45
<211> 17
<212> PRT
<213> Canis familiaris
<400> 45
Phe Asn Glu Cys Arg Cys Thr Asp Thr Pro Pro Cys Pro Val Pro Glu
1 5 10 15
Pro
<210> 46
<211> 22
<212> PRT
<213> Canis familiaris
<400> 46
Pro Lys Arg Glu Asn Gly Arg Val Pro Arg Pro Pro Asp Cys Pro Lys
1 5 10 15
Cys Pro Ala Pro Glu Met
            20
<210> 47
<211> 20
<212> PRT
<213> Canis familiaris
<400> 47
Ala Lys Glu Cys Glu Cys Lys Cys Asn Cys Asn Asn Cys Pro Cys Pro
1 5 10 15
Gly Cys Gly Leu
            20
<210> 48
<211> 17
<212> PRT
<213> Artificial Sequence
<220>
<223> modified canine
<400> 48
Pro Lys Glu Ser Thr Cys Lys Cys Ile Pro Pro Cys Pro Val Pro Glu
1 5 10 15
Ser
<210> 49
<211> 216
<212> PRT
<213> Canis familiaris
<400> 49
Leu Gly Gly Pro Ser Val Leu Ile Phe Pro Pro Lys Pro Lys Asp Ile
1 5 10 15
Leu Arg Ile Thr Arg Thr Pro Glu Val Thr Cys Val Val Leu Asp Leu
                20 25 30
Gly Arg Glu Asp Pro Glu Val Gln Ile Ser Trp Phe Val Asp Gly Lys
          35 40 45
Glu Val His Thr Ala Lys Thr Gln Ser Arg Glu Gln Gln Phe Asn Gly
    50 55 60
Thr Tyr Arg Val Val Ser Val Leu Pro Ile Glu His Gln Asp Trp Leu
65 70 75 80
Thr Gly Lys Glu Phe Lys Cys Arg Val Asn His Ile Asp Leu Pro Ser
                  85 90 95
Pro Ile Glu Arg Thr Ile Ser Lys Ala Arg Gly Arg Ala His Lys Pro
            100 105 110
Ser Val Tyr Val Leu Pro Pro Ser Pro Lys Glu Leu Ser Ser Ser Asp
          115 120 125
Thr Val Ser Ile Thr Cys Leu Ile Lys Asp Phe Tyr Pro Pro Asp Ile
    130 135 140
Asp Val Glu Trp Gln Ser Asn Gly Gln Gln Glu Pro Glu Arg Lys His
145 150 155 160
Arg Met Thr Pro Pro Gln Leu Asp Glu Asp Gly Ser Tyr Phe Leu Tyr
                  165 170 175
Ser Lys Leu Ser Val Asp Lys Ser Arg Trp Gln Gln Gly Asp Pro Phe
              180 185 190
Thr Cys Ala Val Met His Glu Thr Leu Gln Asn His Tyr Thr Asp Leu
          195 200 205
Ser Leu Ser His Ser Pro Gly Lys
    210 215
<210> 50
<211> 215
<212> PRT
<213> Canis familiaris
<400> 50
Leu Gly Gly Pro Ser Val Phe Ile Phe Pro Pro Lys Pro Lys Asp Thr
1 5 10 15
Leu Leu Ile Ala Arg Thr Pro Glu Val Thr Cys Val Val Val Asp Leu
                20 25 30
Asp Pro Glu Asp Pro Glu Val Gln Ile Ser Trp Phe Val Asp Gly Lys
          35 40 45
Gln Met Gln Thr Ala Lys Thr Gln Pro Arg Glu Glu Gln Phe Asn Gly
    50 55 60
Thr Tyr Arg Val Val Ser Val Leu Pro Ile Gly His Gln Asp Trp Leu
65 70 75 80
Lys Gly Lys Gln Phe Thr Cys Lys Val Asn Asn Lys Ala Leu Pro Ser
                  85 90 95
Pro Ile Glu Arg Thr Ile Ser Lys Ala Arg Gly Gln Ala His Gln Pro
            100 105 110
Ser Val Tyr Val Leu Pro Pro Ser Arg Glu Glu Leu Ser Lys Asn Thr
          115 120 125
Val Ser Leu Thr Cys Leu Ile Lys Asp Phe Phe Pro Pro Asp Ile Asp
    130 135 140
Val Glu Trp Gln Ser Asn Gly Gln Gln Glu Pro Glu Ser Lys Tyr Arg
145 150 155 160
Thr Thr Pro Pro Gln Leu Asp Glu Asp Gly Ser Tyr Phe Leu Tyr Ser
                  165 170 175
Lys Leu Ser Val Asp Lys Ser Arg Trp Gln Arg Gly Asp Thr Phe Ile
              180 185 190
Cys Ala Val Met His Glu Ala Leu His Asn His Tyr Thr Gln Glu Ser
          195 200 205
Leu Ser His Ser Pro Gly Lys
    210 215
<210> 51
<211> 214
<212> PRT
<213> Artificial Sequence
<220>
<223> modified canine
<400> 51
Leu Gly Gly Pro Ser Val Phe Ile Phe Pro Pro Lys Pro Lys Asp Thr
1 5 10 15
Leu Leu Ile Ala Arg Thr Pro Glu Val Thr Cys Val Val Val Ala Leu
                20 25 30
Asp Pro Glu Asp Pro Glu Val Gln Ile Ser Trp Phe Val Asp Gly Lys
          35 40 45
Gln Met Gln Thr Ala Lys Thr Gln Pro Arg Glu Glu Gln Phe Ala Gly
    50 55 60
Thr Tyr Arg Val Val Ser Val Leu Pro Ile Gly His Gln Asp Trp Leu
65 70 75 80
Lys Gly Lys Gln Phe Thr Cys Lys Val Asn Asn Lys Ala Leu Pro Ser
                  85 90 95
Pro Ile Glu Arg Thr Ile Ser Lys Ala Arg Gly Gln Ala His Gln Pro
            100 105 110
Ser Val Tyr Val Leu Pro Pro Ser Arg Glu Glu Leu Ser Lys Asn Thr
          115 120 125
Val Ser Leu Thr Cys Leu Ile Lys Asp Phe Phe Pro Pro Asp Ile Asp
    130 135 140
Val Glu Trp Gln Ser Asn Gly Gln Gln Glu Pro Glu Ser Lys Tyr Arg
145 150 155 160
Thr Thr Pro Pro Gln Leu Asp Glu Asp Gly Ser Tyr Phe Leu Tyr Ser
                  165 170 175
Lys Leu Ser Val Asp Lys Ser Arg Trp Gln Arg Gly Asp Thr Phe Ile
              180 185 190
Cys Ala Val Met His Glu Ala Leu His Asn His Tyr Thr Gln Glu Ser
          195 200 205
Leu Ser His Ser Pro Gly
      210
<210> 52
<211> 215
<212> PRT
<213> Canis familiaris
<400> 52
Leu Gly Gly Pro Ser Val Phe Ile Phe Pro Pro Lys Pro Lys Asp Ile
1 5 10 15
Leu Val Thr Ala Arg Thr Pro Thr Val Thr Cys Val Val Val Asp Leu
                20 25 30
Asp Pro Glu Asn Pro Glu Val Gln Ile Ser Trp Phe Val Asp Ser Lys
          35 40 45
Gln Val Gln Thr Ala Asn Thr Gln Pro Arg Glu Glu Gln Ser Asn Gly
    50 55 60
Thr Tyr Arg Val Val Ser Val Leu Pro Ile Gly His Gln Asp Trp Leu
65 70 75 80
Ser Gly Lys Gln Phe Lys Cys Lys Val Asn Asn Lys Ala Leu Pro Ser
                  85 90 95
Pro Ile Glu Glu Ile Ile Ser Lys Thr Pro Gly Gln Ala His Gln Pro
            100 105 110
Asn Val Tyr Val Leu Pro Pro Ser Arg Asp Glu Met Ser Lys Asn Thr
          115 120 125
Val Thr Leu Thr Cys Leu Val Lys Asp Phe Phe Pro Pro Glu Ile Asp
    130 135 140
Val Glu Trp Gln Ser Asn Gly Gln Gln Glu Pro Glu Ser Lys Tyr Arg
145 150 155 160
Met Thr Pro Pro Gln Leu Asp Glu Asp Gly Ser Tyr Phe Leu Tyr Ser
                  165 170 175
Lys Leu Ser Val Asp Lys Ser Arg Trp Gln Arg Gly Asp Thr Phe Ile
              180 185 190
Cys Ala Val Met His Glu Ala Leu His Asn His Tyr Thr Gln Ile Ser
          195 200 205
Leu Ser His Ser Pro Gly Lys
    210 215
<210> 53
<211> 216
<212> PRT
<213> Canis familiaris
<400> 53
Leu Gly Gly Pro Ser Val Phe Ile Phe Pro Pro Lys Pro Lys Asp Ile
1 5 10 15
Leu Arg Ile Thr Arg Thr Pro Glu Ile Thr Cys Val Val Leu Asp Leu
                20 25 30
Gly Arg Glu Asp Pro Glu Val Gln Ile Ser Trp Phe Val Asp Gly Lys
          35 40 45
Glu Val His Thr Ala Lys Thr Gln Pro Arg Glu Gln Gln Phe Asn Ser
    50 55 60
Thr Tyr Arg Val Val Ser Val Leu Pro Ile Glu His Gln Asp Trp Leu
65 70 75 80
Thr Gly Lys Glu Phe Lys Cys Arg Val Asn His Ile Gly Leu Pro Ser
                  85 90 95
Pro Ile Glu Arg Thr Ile Ser Lys Ala Arg Gly Gln Ala His Gln Pro
            100 105 110
Ser Val Tyr Val Leu Pro Pro Ser Pro Lys Glu Leu Ser Ser Ser Asp
          115 120 125
Thr Val Thr Leu Thr Cys Leu Ile Lys Asp Phe Phe Pro Pro Glu Ile
    130 135 140
Asp Val Glu Trp Gln Ser Asn Gly Gln Pro Glu Pro Glu Ser Lys Tyr
145 150 155 160
His Thr Thr Ala Pro Gln Leu Asp Glu Asp Gly Ser Tyr Phe Leu Tyr
                  165 170 175
Ser Lys Leu Ser Val Asp Lys Ser Arg Trp Gln Gln Gly Asp Thr Phe
              180 185 190
Thr Cys Ala Val Met His Glu Ala Leu Gln Asn His Tyr Thr Asp Leu
          195 200 205
Ser Leu Ser His Ser Pro Gly Lys
    210 215
<210> 54
<211> 335
<212> PRT
<213> Felis catus
<400> 54
Ala Ser Thr Thr Ala Pro Ser Val Phe Pro Leu Ala Pro Ser Cys Gly
1 5 10 15
Thr Thr Ser Gly Ala Thr Val Ala Leu Ala Cys Leu Val Leu Gly Tyr
                20 25 30
Phe Pro Glu Pro Val Thr Val Ser Trp Asn Ser Gly Ala Leu Thr Ser
          35 40 45
Gly Val His Thr Phe Pro Ala Val Leu Gln Ala Ser Gly Leu Tyr Ser
    50 55 60
Leu Ser Ser Met Val Thr Val Pro Ser Ser Arg Trp Leu Ser Asp Thr
65 70 75 80
Phe Thr Cys Asn Val Ala His Pro Pro Ser Asn Thr Lys Val Asp Lys
                  85 90 95
Thr Val Arg Lys Thr Asp His Pro Pro Gly Pro Lys Pro Cys Asp Cys
            100 105 110
Pro Lys Cys Pro Pro Pro Glu Met Leu Gly Gly Pro Ser Ile Phe Ile
          115 120 125
Phe Pro Pro Lys Pro Lys Asp Thr Leu Ser Ile Ser Arg Thr Pro Glu
    130 135 140
Val Thr Cys Leu Val Val Asp Leu Gly Pro Asp Asp Ser Asp Val Gln
145 150 155 160
Ile Thr Trp Phe Val Asp Asn Thr Gln Val Tyr Thr Ala Lys Thr Ser
                  165 170 175
Pro Arg Glu Glu Gln Phe Asn Ser Thr Tyr Arg Val Val Ser Val Leu
              180 185 190
Pro Ile Leu His Gln Asp Trp Leu Lys Gly Lys Glu Phe Lys Cys Lys
          195 200 205
Val Asn Ser Lys Ser Leu Pro Ser Pro Ile Glu Arg Thr Ile Ser Lys
    210 215 220
Ala Lys Gly Gln Pro His Glu Pro Gln Val Tyr Val Leu Pro Pro Ala
225 230 235 240
Gln Glu Glu Leu Ser Arg Asn Lys Val Ser Val Thr Cys Leu Ile Lys
                    245 250 255
Ser Phe His Pro Pro Asp Ile Ala Val Glu Trp Glu Ile Thr Gly Gln
            260 265 270
Pro Glu Pro Glu Asn Asn Tyr Arg Thr Thr Pro Pro Gln Leu Asp Ser
        275 280 285
Asp Gly Thr Tyr Phe Val Tyr Ser Lys Leu Ser Val Asp Arg Ser His
  290 295 300
Trp Gln Arg Gly Asn Thr Tyr Thr Cys Ser Val Ser His Glu Ala Leu
305 310 315 320
His Ser His His Thr Gln Lys Ser Leu Thr Gln Ser Pro Gly Lys
                    325 330 335
<210> 55
<211> 335
<212> PRT
<213> Felis catus
<400> 55
Ala Ser Thr Thr Ala Pro Ser Val Phe Pro Leu Ala Pro Ser Cys Gly
1 5 10 15
Thr Thr Ser Gly Ala Thr Val Ala Leu Ala Cys Leu Val Leu Gly Tyr
                20 25 30
Phe Pro Glu Pro Val Thr Val Ser Trp Asn Ser Gly Ala Leu Thr Ser
          35 40 45
Gly Val His Thr Phe Pro Ala Val Leu Gln Ala Ser Gly Leu Tyr Ser
    50 55 60
Leu Ser Ser Met Val Thr Val Pro Ser Ser Arg Trp Leu Ser Asp Thr
65 70 75 80
Phe Thr Cys Asn Val Ala His Pro Pro Ser Asn Thr Lys Val Asp Lys
                  85 90 95
Thr Val Arg Lys Thr Asp His Pro Pro Gly Pro Lys Pro Cys Asp Cys
            100 105 110
Pro Lys Cys Pro Pro Pro Glu Met Leu Gly Gly Pro Ser Ile Phe Ile
          115 120 125
Phe Pro Pro Lys Pro Lys Asp Thr Leu Ser Ile Ser Arg Thr Pro Glu
    130 135 140
Val Thr Cys Leu Val Val Asp Leu Gly Pro Asp Asp Ser Asp Val Gln
145 150 155 160
Ile Thr Trp Phe Val Asp Asn Thr Gln Val Tyr Thr Ala Lys Thr Ser
                  165 170 175
Pro Arg Glu Glu Gln Phe Asn Ser Thr Tyr Arg Val Val Ser Val Leu
              180 185 190
Pro Ile Leu His Gln Asp Trp Leu Lys Gly Lys Glu Phe Lys Cys Lys
          195 200 205
Val Asn Ser Lys Ser Leu Pro Ser Pro Ile Glu Arg Thr Ile Ser Lys
    210 215 220
Asp Lys Gly Gln Pro His Glu Pro Gln Val Tyr Val Leu Pro Pro Ala
225 230 235 240
Gln Glu Glu Leu Ser Arg Asn Lys Val Ser Val Thr Cys Leu Ile Glu
                    245 250 255
Gly Phe Tyr Pro Ser Asp Ile Ala Val Glu Trp Glu Ile Thr Gly Gln
            260 265 270
Pro Glu Pro Glu Asn Asn Tyr Arg Thr Thr Pro Pro Gln Leu Asp Ser
        275 280 285
Asp Gly Thr Tyr Phe Leu Tyr Ser Arg Leu Ser Val Asp Arg Ser Arg
  290 295 300
Trp Gln Arg Gly Asn Thr Tyr Thr Cys Ser Val Ser His Glu Ala Leu
305 310 315 320
His Ser His His Thr Gln Lys Ser Leu Thr Gln Ser Pro Gly Lys
                    325 330 335
<210> 56
<211> 335
<212> PRT
<213> Felis catus
<400> 56
Ala Ser Thr Thr Ala Ser Ser Val Phe Pro Leu Ala Pro Ser Cys Gly
1 5 10 15
Thr Thr Ser Gly Ala Thr Val Ala Leu Ala Cys Leu Val Leu Gly Tyr
                20 25 30
Phe Pro Glu Pro Val Thr Val Ser Trp Asn Ser Gly Ala Leu Thr Ser
          35 40 45
Gly Val His Thr Phe Pro Ser Val Leu Gln Ala Ser Gly Leu Tyr Ser
    50 55 60
Leu Ser Ser Met Val Thr Val Pro Ser Ser Arg Trp Leu Ser Asp Thr
65 70 75 80
Phe Thr Cys Asn Val Ala His Arg Pro Ser Ser Thr Lys Val Asp Lys
                  85 90 95
Thr Val Pro Lys Thr Ala Ser Thr Ile Glu Ser Lys Thr Gly Glu Gly
            100 105 110
Pro Lys Cys Pro Val Pro Glu Ile Pro Gly Ala Pro Ser Val Phe Ile
          115 120 125
Phe Pro Pro Lys Pro Lys Asp Thr Leu Ser Ile Ser Arg Thr Pro Glu
    130 135 140
Val Thr Cys Leu Val Val Asp Leu Gly Pro Asp Asp Ser Asn Val Gln
145 150 155 160
Ile Thr Trp Phe Val Asp Asn Thr Glu Met His Thr Ala Lys Thr Arg
                  165 170 175
Pro Arg Glu Glu Gln Phe Asn Ser Thr Tyr Arg Val Val Ser Val Leu
              180 185 190
Pro Ile Leu His Gln Asp Trp Leu Lys Gly Lys Glu Phe Lys Cys Lys
          195 200 205
Val Asn Ser Lys Ser Leu Pro Ser Ala Met Glu Arg Thr Ile Ser Lys
    210 215 220
Ala Lys Gly Gln Pro His Glu Pro Gln Val Tyr Val Leu Pro Pro Thr
225 230 235 240
Gln Glu Glu Leu Ser Glu Asn Lys Val Ser Val Thr Cys Leu Ile Lys
                    245 250 255
Gly Phe His Pro Pro Asp Ile Ala Val Glu Trp Glu Ile Thr Gly Gln
            260 265 270
Pro Glu Pro Glu Asn Asn Tyr Gln Thr Thr Pro Pro Gln Leu Asp Ser
        275 280 285
Asp Gly Thr Tyr Phe Leu Tyr Ser Arg Leu Ser Val Asp Arg Ser His
  290 295 300
Trp Gln Arg Gly Asn Thr Tyr Thr Cys Ser Val Ser His Glu Ala Leu
305 310 315 320
His Ser His His Thr Gln Lys Ser Leu Thr Gln Ser Pro Gly Lys
                    325 330 335
<210> 57
<211> 123
<212> PRT
<213> Artificial Sequence
<220>
<223> Felinized human
<400> 57
Asp Val Gln Leu Val Glu Ser Gly Gly Asp Leu Val Gln Pro Gly Gly
1 5 10 15
Ser Leu Arg Leu Thr Cys Ala Ala Ser Gly Phe Thr Leu Arg Ser Tyr
                20 25 30
Ser Met Asn Trp Val Arg Gln Ala Pro Gly Lys Gly Leu Gln Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Thr Leu Tyr
65 70 75 80
Leu Gln Met Asn Ser Leu Lys Thr Glu Asp Thr Ala Thr Tyr Tyr Cys
                  85 90 95
Ala Lys Val Tyr Ser Ser Gly Trp His Val Ser Asp Tyr Phe Asp Tyr
            100 105 110
Trp Gly Gln Gly Ile Leu Val Thr Val Ser Ser
          115 120
<210> 58
<211> 123
<212> PRT
<213> Artificial Sequence
<220>
<223> Felinized human
<400> 58
Glu Val Gln Leu Val Glu Ser Gly Gly Asp Leu Val Gln Pro Gly Gly
1 5 10 15
Ser Leu Arg Leu Thr Cys Val Ala Ser Gly Phe Thr Phe Ser Ser Tyr
                20 25 30
Ser Met Asn Trp Val Arg Gln Ala Pro Gly Lys Gly Leu Gln Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Thr Leu Tyr
65 70 75 80
Leu Gln Met Asp Ser Leu Lys Asp Glu Asp Thr Ala Thr Tyr Tyr Cys
                  85 90 95
Ala Arg Val Tyr Ser Ser Gly Trp His Val Ser Asp Tyr Phe Asp Tyr
            100 105 110
Trp Gly Gln Gly Ile Leu Val Thr Val Ser Ser
          115 120
<210> 59
<211> 123
<212> PRT
<213> Artificial Sequence
<220>
<223> Felinized human
<400> 59
Glu Val Gln Leu Val Glu Ser Glu Gly Asp Leu Val Gln Pro Gly Gly
1 5 10 15
Ser Leu Arg Leu Thr Cys Ala Ala Ser Gly Phe Thr Leu Arg Ser Tyr
                20 25 30
Ser Met Asn Trp Val Arg Gln Ala Pro Gly Lys Gly Leu Gln Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Thr Leu Tyr
65 70 75 80
Leu Gln Met Asn Asn Leu Lys Thr Glu Asp Thr Ala Thr Tyr Tyr Cys
                  85 90 95
Ala Lys Val Tyr Ser Ser Gly Trp His Val Ser Asp Tyr Phe Asp Tyr
            100 105 110
Trp Gly Gln Gly Ile Leu Val Thr Val Ser Ser
          115 120
<210> 60
<211> 123
<212> PRT
<213> Artificial Sequence
<220>
<223> Felinized human
<400> 60
Glu Val Gln Leu Val Glu Ser Glu Gly Asp Leu Val Gln Pro Gly Gly
1 5 10 15
Ser Leu Arg Leu Thr Cys Val Ala Ser Gly Phe Thr Phe Ser Ser Tyr
                20 25 30
Ser Met Asn Trp Val Arg Gln Ala Pro Gly Lys Gly Leu Gln Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Thr Leu Tyr
65 70 75 80
Leu Gln Met Asp Ser Leu Lys Asp Glu Asp Thr Ala Thr Tyr Tyr Cys
                  85 90 95
Ala Arg Val Tyr Ser Ser Gly Trp His Val Ser Asp Tyr Phe Asp Tyr
            100 105 110
Trp Gly Gln Gly Ile Leu Val Thr Val Ser Ser
          115 120
<210> 61
<211> 107
<212> PRT
<213> Artificial Sequence
<220>
<223> Felinized human
<400> 61
Glu Ile Gln Leu Thr Gln Ser Pro Ser Ser Leu Ser Ala Ser Pro Gly
1 5 10 15
Asp Arg Val Thr Ile Thr Cys Arg Ala Ser Gln Gly Ile Ser Ser Ala
                20 25 30
Leu Ala Trp Tyr Gln Gln Lys Pro Gly Lys Val Pro Lys Leu Leu Ile
          35 40 45
Tyr Asp Ala Ser Ser Leu Glu Ser Gly Val Pro Ser Arg Phe Ser Gly
    50 55 60
Ser Gly Ser Gly Thr Asp Phe Thr Leu Thr Ile Ser Ser Leu Glu Pro
65 70 75 80
Glu Asp Ala Ala Thr Tyr Tyr Cys Gln Gln Phe Asn Ser Tyr Pro Leu
                  85 90 95
Thr Phe Gly Gln Gly Thr Lys Leu Glu Ile Lys
            100 105
<210> 62
<211> 107
<212> PRT
<213> Artificial Sequence
<220>
<223> Felinized human
<400> 62
Ala Ile Thr Leu Thr Gln Ser Pro Gly Ser Leu Ala Gly Ser Pro Gly
1 5 10 15
Gln Gln Val Thr Ile Thr Cys Arg Ala Ser Gln Gly Ile Ser Ser Ala
                20 25 30
Leu Ala Trp Tyr Gln Gln Lys Pro Gly Gln His Pro Lys Leu Leu Ile
          35 40 45
Tyr Asp Ala Ser Ser Leu Glu Ser Gly Val Pro Asp Arg Phe Ser Gly
    50 55 60
Ser Gly Ser Gly Thr Asp Phe Thr Leu Thr Ile Ser Asn Leu Gln Ala
65 70 75 80
Glu Asp Val Ala Ser Tyr Tyr Cys Gln Gln Phe Asn Ser Tyr Pro Leu
                  85 90 95
Thr Phe Gly Gln Gly Thr Lys Leu Glu Ile Lys
            100 105
<210> 63
<211> 458
<212> PRT
<213> Artificial Sequence
<220>
<223> Human-Feline fusion protein
<400> 63
Glu Val Gln Leu Val Glu Ser Gly Gly Gly Leu Val Gln Pro Gly Gly
1 5 10 15
Ser Leu Arg Leu Ser Cys Ala Ala Ser Gly Phe Thr Leu Arg Ser Tyr
                20 25 30
Ser Met Asn Trp Val Arg Gln Ala Pro Gly Lys Gly Leu Glu Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Ser Leu Tyr
65 70 75 80
Leu Gln Met Asp Ser Leu Arg Asp Glu Asp Thr Ala Met Tyr Tyr Cys
                  85 90 95
Ala Arg Val Tyr Ser Ser Gly Trp His Val Ser Asp Tyr Phe Asp Tyr
            100 105 110
Trp Gly Gln Gly Ile Leu Val Thr Val Ser Ser Ala Ser Thr Thr Ala
          115 120 125
Pro Ser Val Phe Pro Leu Ala Pro Ser Cys Gly Thr Thr Ser Gly Ala
    130 135 140
Thr Val Ala Leu Ala Cys Leu Val Leu Gly Tyr Phe Pro Glu Pro Val
145 150 155 160
Thr Val Ser Trp Asn Ser Gly Ala Leu Thr Ser Gly Val His Thr Phe
                  165 170 175
Pro Ala Val Leu Gln Ala Ser Gly Leu Tyr Ser Leu Ser Ser Met Val
              180 185 190
Thr Val Pro Ser Ser Arg Trp Leu Ser Asp Thr Phe Thr Cys Asn Val
          195 200 205
Ala His Pro Pro Ser Asn Thr Lys Val Asp Lys Thr Val Arg Lys Thr
    210 215 220
Asp His Pro Pro Gly Pro Lys Pro Cys Asp Cys Pro Lys Cys Pro Pro
225 230 235 240
Pro Glu Met Leu Gly Gly Pro Ser Ile Phe Ile Phe Pro Pro Lys Pro
                    245 250 255
Lys Asp Thr Leu Ser Ile Ser Arg Thr Pro Glu Val Thr Cys Leu Val
            260 265 270
Val Ala Leu Gly Pro Asp Asp Ser Asp Val Gln Ile Thr Trp Phe Val
        275 280 285
Asp Asn Thr Gln Val Tyr Thr Ala Lys Thr Ser Pro Arg Glu Glu Gln
  290 295 300
Phe Ala Ser Thr Tyr Arg Val Val Ser Val Leu Pro Ile Leu His Gln
305 310 315 320
Asp Trp Leu Lys Gly Lys Glu Phe Lys Cys Lys Val Asn Ser Lys Ser
                    325 330 335
Leu Pro Ser Pro Ile Glu Arg Thr Ile Ser Lys Ala Lys Gly Gln Pro
              340 345 350
His Glu Pro Gln Val Tyr Val Leu Pro Pro Ala Gln Glu Glu Leu Ser
          355 360 365
Arg Asn Lys Val Ser Val Thr Cys Leu Ile Lys Ser Phe His Pro Pro
    370 375 380
Asp Ile Ala Val Glu Trp Glu Ile Thr Gly Gln Pro Glu Pro Glu Asn
385 390 395 400
Asn Tyr Arg Thr Thr Pro Pro Gln Leu Asp Ser Asp Gly Thr Tyr Phe
                    405 410 415
Val Tyr Ser Lys Leu Ser Val Asp Arg Ser His Trp Gln Arg Gly Asn
              420 425 430
Thr Tyr Thr Cys Ser Val Ser His Glu Ala Leu His Ser His His Thr
          435 440 445
Gln Lys Ser Leu Thr Gln Ser Pro Gly Lys
    450 455
<210> 64
<211> 458
<212> PRT
<213> Artificial Sequence
<220>
<223> Felinized human
<400> 64
Asp Val Gln Leu Val Glu Ser Gly Gly Asp Leu Val Gln Pro Gly Gly
1 5 10 15
Ser Leu Arg Leu Thr Cys Ala Ala Ser Gly Phe Thr Leu Arg Ser Tyr
                20 25 30
Ser Met Asn Trp Val Arg Gln Ala Pro Gly Lys Gly Leu Gln Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Thr Leu Tyr
65 70 75 80
Leu Gln Met Asn Ser Leu Lys Thr Glu Asp Thr Ala Thr Tyr Tyr Cys
                  85 90 95
Ala Lys Val Tyr Ser Ser Gly Trp His Val Ser Asp Tyr Phe Asp Tyr
            100 105 110
Trp Gly Gln Gly Ile Leu Val Thr Val Ser Ser Ala Ser Thr Thr Ala
          115 120 125
Pro Ser Val Phe Pro Leu Ala Pro Ser Cys Gly Thr Thr Ser Gly Ala
    130 135 140
Thr Val Ala Leu Ala Cys Leu Val Leu Gly Tyr Phe Pro Glu Pro Val
145 150 155 160
Thr Val Ser Trp Asn Ser Gly Ala Leu Thr Ser Gly Val His Thr Phe
                  165 170 175
Pro Ala Val Leu Gln Ala Ser Gly Leu Tyr Ser Leu Ser Ser Met Val
              180 185 190
Thr Val Pro Ser Ser Arg Trp Leu Ser Asp Thr Phe Thr Cys Asn Val
          195 200 205
Ala His Pro Pro Ser Asn Thr Lys Val Asp Lys Thr Val Arg Lys Thr
    210 215 220
Asp His Pro Pro Gly Pro Lys Pro Cys Asp Cys Pro Lys Cys Pro Pro
225 230 235 240
Pro Glu Met Leu Gly Gly Pro Ser Ile Phe Ile Phe Pro Pro Lys Pro
                    245 250 255
Lys Asp Thr Leu Ser Ile Ser Arg Thr Pro Glu Val Thr Cys Leu Val
            260 265 270
Val Ala Leu Gly Pro Asp Asp Ser Asp Val Gln Ile Thr Trp Phe Val
        275 280 285
Asp Asn Thr Gln Val Tyr Thr Ala Lys Thr Ser Pro Arg Glu Glu Gln
  290 295 300
Phe Ala Ser Thr Tyr Arg Val Val Ser Val Leu Pro Ile Leu His Gln
305 310 315 320
Asp Trp Leu Lys Gly Lys Glu Phe Lys Cys Lys Val Asn Ser Lys Ser
                    325 330 335
Leu Pro Ser Pro Ile Glu Arg Thr Ile Ser Lys Ala Lys Gly Gln Pro
              340 345 350
His Glu Pro Gln Val Tyr Val Leu Pro Pro Ala Gln Glu Glu Leu Ser
          355 360 365
Arg Asn Lys Val Ser Val Thr Cys Leu Ile Lys Ser Phe His Pro Pro
    370 375 380
Asp Ile Ala Val Glu Trp Glu Ile Thr Gly Gln Pro Glu Pro Glu Asn
385 390 395 400
Asn Tyr Arg Thr Thr Pro Pro Gln Leu Asp Ser Asp Gly Thr Tyr Phe
                    405 410 415
Val Tyr Ser Lys Leu Ser Val Asp Arg Ser His Trp Gln Arg Gly Asn
              420 425 430
Thr Tyr Thr Cys Ser Val Ser His Glu Ala Leu His Ser His His Thr
          435 440 445
Gln Lys Ser Leu Thr Gln Ser Pro Gly Lys
    450 455
<210> 65
<211> 458
<212> PRT
<213> Artificial Sequence
<220>
<223> Felinized human
<400> 65
Glu Val Gln Leu Val Glu Ser Gly Gly Asp Leu Val Gln Pro Gly Gly
1 5 10 15
Ser Leu Arg Leu Thr Cys Val Ala Ser Gly Phe Thr Phe Ser Ser Tyr
                20 25 30
Ser Met Asn Trp Val Arg Gln Ala Pro Gly Lys Gly Leu Gln Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Thr Leu Tyr
65 70 75 80
Leu Gln Met Asp Ser Leu Lys Asp Glu Asp Thr Ala Thr Tyr Tyr Cys
                  85 90 95
Ala Arg Val Tyr Ser Ser Gly Trp His Val Ser Asp Tyr Phe Asp Tyr
            100 105 110
Trp Gly Gln Gly Ile Leu Val Thr Val Ser Ser Ala Ser Thr Thr Ala
          115 120 125
Pro Ser Val Phe Pro Leu Ala Pro Ser Cys Gly Thr Thr Ser Gly Ala
    130 135 140
Thr Val Ala Leu Ala Cys Leu Val Leu Gly Tyr Phe Pro Glu Pro Val
145 150 155 160
Thr Val Ser Trp Asn Ser Gly Ala Leu Thr Ser Gly Val His Thr Phe
                  165 170 175
Pro Ala Val Leu Gln Ala Ser Gly Leu Tyr Ser Leu Ser Ser Met Val
              180 185 190
Thr Val Pro Ser Ser Arg Trp Leu Ser Asp Thr Phe Thr Cys Asn Val
          195 200 205
Ala His Pro Pro Ser Asn Thr Lys Val Asp Lys Thr Val Arg Lys Thr
    210 215 220
Asp His Pro Pro Gly Pro Lys Pro Cys Asp Cys Pro Lys Cys Pro Pro
225 230 235 240
Pro Glu Met Leu Gly Gly Pro Ser Ile Phe Ile Phe Pro Pro Lys Pro
                    245 250 255
Lys Asp Thr Leu Ser Ile Ser Arg Thr Pro Glu Val Thr Cys Leu Val
            260 265 270
Val Ala Leu Gly Pro Asp Asp Ser Asp Val Gln Ile Thr Trp Phe Val
        275 280 285
Asp Asn Thr Gln Val Tyr Thr Ala Lys Thr Ser Pro Arg Glu Glu Gln
  290 295 300
Phe Ala Ser Thr Tyr Arg Val Val Ser Val Leu Pro Ile Leu His Gln
305 310 315 320
Asp Trp Leu Lys Gly Lys Glu Phe Lys Cys Lys Val Asn Ser Lys Ser
                    325 330 335
Leu Pro Ser Pro Ile Glu Arg Thr Ile Ser Lys Ala Lys Gly Gln Pro
              340 345 350
His Glu Pro Gln Val Tyr Val Leu Pro Pro Ala Gln Glu Glu Leu Ser
          355 360 365
Arg Asn Lys Val Ser Val Thr Cys Leu Ile Lys Ser Phe His Pro Pro
    370 375 380
Asp Ile Ala Val Glu Trp Glu Ile Thr Gly Gln Pro Glu Pro Glu Asn
385 390 395 400
Asn Tyr Arg Thr Thr Pro Pro Gln Leu Asp Ser Asp Gly Thr Tyr Phe
                    405 410 415
Val Tyr Ser Lys Leu Ser Val Asp Arg Ser His Trp Gln Arg Gly Asn
              420 425 430
Thr Tyr Thr Cys Ser Val Ser His Glu Ala Leu His Ser His His Thr
          435 440 445
Gln Lys Ser Leu Thr Gln Ser Pro Gly Lys
    450 455
<210> 66
<211> 458
<212> PRT
<213> Artificial Sequence
<220>
<223> Felinized human
<400> 66
Glu Val Gln Leu Val Glu Ser Glu Gly Asp Leu Val Gln Pro Gly Gly
1 5 10 15
Ser Leu Arg Leu Thr Cys Ala Ala Ser Gly Phe Thr Leu Arg Ser Tyr
                20 25 30
Ser Met Asn Trp Val Arg Gln Ala Pro Gly Lys Gly Leu Gln Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Thr Leu Tyr
65 70 75 80
Leu Gln Met Asn Asn Leu Lys Thr Glu Asp Thr Ala Thr Tyr Tyr Cys
                  85 90 95
Ala Lys Val Tyr Ser Ser Gly Trp His Val Ser Asp Tyr Phe Asp Tyr
            100 105 110
Trp Gly Gln Gly Ile Leu Val Thr Val Ser Ser Ala Ser Thr Thr Ala
          115 120 125
Pro Ser Val Phe Pro Leu Ala Pro Ser Cys Gly Thr Thr Ser Gly Ala
    130 135 140
Thr Val Ala Leu Ala Cys Leu Val Leu Gly Tyr Phe Pro Glu Pro Val
145 150 155 160
Thr Val Ser Trp Asn Ser Gly Ala Leu Thr Ser Gly Val His Thr Phe
                  165 170 175
Pro Ala Val Leu Gln Ala Ser Gly Leu Tyr Ser Leu Ser Ser Met Val
              180 185 190
Thr Val Pro Ser Ser Arg Trp Leu Ser Asp Thr Phe Thr Cys Asn Val
          195 200 205
Ala His Pro Pro Ser Asn Thr Lys Val Asp Lys Thr Val Arg Lys Thr
    210 215 220
Asp His Pro Pro Gly Pro Lys Pro Cys Asp Cys Pro Lys Cys Pro Pro
225 230 235 240
Pro Glu Met Leu Gly Gly Pro Ser Ile Phe Ile Phe Pro Pro Lys Pro
                    245 250 255
Lys Asp Thr Leu Ser Ile Ser Arg Thr Pro Glu Val Thr Cys Leu Val
            260 265 270
Val Ala Leu Gly Pro Asp Asp Ser Asp Val Gln Ile Thr Trp Phe Val
        275 280 285
Asp Asn Thr Gln Val Tyr Thr Ala Lys Thr Ser Pro Arg Glu Glu Gln
  290 295 300
Phe Ala Ser Thr Tyr Arg Val Val Ser Val Leu Pro Ile Leu His Gln
305 310 315 320
Asp Trp Leu Lys Gly Lys Glu Phe Lys Cys Lys Val Asn Ser Lys Ser
                    325 330 335
Leu Pro Ser Pro Ile Glu Arg Thr Ile Ser Lys Ala Lys Gly Gln Pro
              340 345 350
His Glu Pro Gln Val Tyr Val Leu Pro Pro Ala Gln Glu Glu Leu Ser
          355 360 365
Arg Asn Lys Val Ser Val Thr Cys Leu Ile Lys Ser Phe His Pro Pro
    370 375 380
Asp Ile Ala Val Glu Trp Glu Ile Thr Gly Gln Pro Glu Pro Glu Asn
385 390 395 400
Asn Tyr Arg Thr Thr Pro Pro Gln Leu Asp Ser Asp Gly Thr Tyr Phe
                    405 410 415
Val Tyr Ser Lys Leu Ser Val Asp Arg Ser His Trp Gln Arg Gly Asn
              420 425 430
Thr Tyr Thr Cys Ser Val Ser His Glu Ala Leu His Ser His His Thr
          435 440 445
Gln Lys Ser Leu Thr Gln Ser Pro Gly Lys
    450 455
<210> 67
<211> 458
<212> PRT
<213> Artificial Sequence
<220>
<223> Felinized human
<400> 67
Glu Val Gln Leu Val Glu Ser Glu Gly Asp Leu Val Gln Pro Gly Gly
1 5 10 15
Ser Leu Arg Leu Thr Cys Val Ala Ser Gly Phe Thr Phe Ser Ser Tyr
                20 25 30
Ser Met Asn Trp Val Arg Gln Ala Pro Gly Lys Gly Leu Gln Trp Val
          35 40 45
Ser Tyr Ile Ser Arg Ser Ser His Thr Ile Phe Tyr Ala Asp Ser Val
    50 55 60
Lys Gly Arg Phe Thr Ile Ser Arg Asp Asn Ala Lys Asn Thr Leu Tyr
65 70 75 80
Leu Gln Met Asp Ser Leu Lys Asp Glu Asp Thr Ala Thr Tyr Tyr Cys
                  85 90 95
Ala Arg Val Tyr Ser Ser Gly Trp His Val Ser Asp Tyr Phe Asp Tyr
            100 105 110
Trp Gly Gln Gly Ile Leu Val Thr Val Ser Ser Ala Ser Thr Thr Ala
          115 120 125
Pro Ser Val Phe Pro Leu Ala Pro Ser Cys Gly Thr Thr Ser Gly Ala
    130 135 140
Thr Val Ala Leu Ala Cys Leu Val Leu Gly Tyr Phe Pro Glu Pro Val
145 150 155 160
Thr Val Ser Trp Asn Ser Gly Ala Leu Thr Ser Gly Val His Thr Phe
                  165 170 175
Pro Ala Val Leu Gln Ala Ser Gly Leu Tyr Ser Leu Ser Ser Met Val
              180 185 190
Thr Val Pro Ser Ser Arg Trp Leu Ser Asp Thr Phe Thr Cys Asn Val
          195 200 205
Ala His Pro Pro Ser Asn Thr Lys Val Asp Lys Thr Val Arg Lys Thr
    210 215 220
Asp His Pro Pro Gly Pro Lys Pro Cys Asp Cys Pro Lys Cys Pro Pro
225 230 235 240
Pro Glu Met Leu Gly Gly Pro Ser Ile Phe Ile Phe Pro Pro Lys Pro
                    245 250 255
Lys Asp Thr Leu Ser Ile Ser Arg Thr Pro Glu Val Thr Cys Leu Val
            260 265 270
Val Ala Leu Gly Pro Asp Asp Ser Asp Val Gln Ile Thr Trp Phe Val
        275 280 285
Asp Asn Thr Gln Val Tyr Thr Ala Lys Thr Ser Pro Arg Glu Glu Gln
  290 295 300
Phe Ala Ser Thr Tyr Arg Val Val Ser Val Leu Pro Ile Leu His Gln
305 310 315 320
Asp Trp Leu Lys Gly Lys Glu Phe Lys Cys Lys Val Asn Ser Lys Ser
                    325 330 335
Leu Pro Ser Pro Ile Glu Arg Thr Ile Ser Lys Ala Lys Gly Gln Pro
              340 345 350
His Glu Pro Gln Val Tyr Val Leu Pro Pro Ala Gln Glu Glu Leu Ser
          355 360 365
Arg Asn Lys Val Ser Val Thr Cys Leu Ile Lys Ser Phe His Pro Pro
    370 375 380
Asp Ile Ala Val Glu Trp Glu Ile Thr Gly Gln Pro Glu Pro Glu Asn
385 390 395 400
Asn Tyr Arg Thr Thr Pro Pro Gln Leu Asp Ser Asp Gly Thr Tyr Phe
                    405 410 415
Val Tyr Ser Lys Leu Ser Val Asp Arg Ser His Trp Gln Arg Gly Asn
              420 425 430
Thr Tyr Thr Cys Ser Val Ser His Glu Ala Leu His Ser His His Thr
          435 440 445
Gln Lys Ser Leu Thr Gln Ser Pro Gly Lys
    450 455
<210> 68
<211> 217
<212> PRT
<213> Artificial Sequence
<220>
<223> Felinized human
<400> 68
Glu Ile Gln Leu Thr Gln Ser Pro Ser Ser Leu Ser Ala Ser Pro Gly
1 5 10 15
Asp Arg Val Thr Ile Thr Cys Arg Ala Ser Gln Gly Ile Ser Ser Ala
                20 25 30
Leu Ala Trp Tyr Gln Gln Lys Pro Gly Lys Val Pro Lys Leu Leu Ile
          35 40 45
Tyr Asp Ala Ser Ser Leu Glu Ser Gly Val Pro Ser Arg Phe Ser Gly
    50 55 60
Ser Gly Ser Gly Thr Asp Phe Thr Leu Thr Ile Ser Ser Leu Glu Pro
65 70 75 80
Glu Asp Ala Ala Thr Tyr Tyr Cys Gln Gln Phe Asn Ser Tyr Pro Leu
                  85 90 95
Thr Phe Gly Gln Gly Thr Lys Leu Glu Ile Lys Arg Ser Asp Ala Gln
            100 105 110
Pro Ser Val Phe Leu Phe Gln Pro Ser Leu Asp Glu Leu His Thr Gly
          115 120 125
Ser Ala Ser Ile Val Cys Ile Leu Asn Asp Phe Tyr Pro Lys Glu Val
    130 135 140
Asn Val Lys Trp Lys Val Asp Gly Val Val Gln Asn Lys Gly Ile Gln
145 150 155 160
Glu Ser Thr Thr Glu Gln Asn Ser Lys Asp Ser Thr Tyr Ser Leu Ser
                  165 170 175
Ser Thr Leu Thr Met Ser Ser Thr Glu Tyr Gln Ser His Glu Lys Phe
              180 185 190
Ser Cys Glu Val Thr His Lys Ser Leu Ala Ser Thr Leu Val Lys Ser
          195 200 205
Phe Asn Arg Ser Glu Cys Gln Arg Glu
    210 215
<210> 69
<211> 217
<212> PRT
<213> Artificial Sequence
<220>
<223> Felinized human
<400> 69
Ala Ile Thr Leu Thr Gln Ser Pro Gly Ser Leu Ala Gly Ser Pro Gly
1 5 10 15
Gln Gln Val Thr Ile Thr Cys Arg Ala Ser Gln Gly Ile Ser Ser Ala
                20 25 30
Leu Ala Trp Tyr Gln Gln Lys Pro Gly Gln His Pro Lys Leu Leu Ile
          35 40 45
Tyr Asp Ala Ser Ser Leu Glu Ser Gly Val Pro Asp Arg Phe Ser Gly
    50 55 60
Ser Gly Ser Gly Thr Asp Phe Thr Leu Thr Ile Ser Asn Leu Gln Ala
65 70 75 80
Glu Asp Val Ala Ser Tyr Tyr Cys Gln Gln Phe Asn Ser Tyr Pro Leu
                  85 90 95
Thr Phe Gly Gln Gly Thr Lys Leu Glu Ile Lys Arg Ser Asp Ala Gln
            100 105 110
Pro Ser Val Phe Leu Phe Gln Pro Ser Leu Asp Glu Leu His Thr Gly
          115 120 125
Ser Ala Ser Ile Val Cys Ile Leu Asn Asp Phe Tyr Pro Lys Glu Val
    130 135 140
Asn Val Lys Trp Lys Val Asp Gly Val Val Gln Asn Lys Gly Ile Gln
145 150 155 160
Glu Ser Thr Thr Glu Gln Asn Ser Lys Asp Ser Thr Tyr Ser Leu Ser
                  165 170 175
Ser Thr Leu Thr Met Ser Ser Thr Glu Tyr Gln Ser His Glu Lys Phe
              180 185 190
Ser Cys Glu Val Thr His Lys Ser Leu Ala Ser Thr Leu Val Lys Ser
          195 200 205
Phe Asn Arg Ser Glu Cys Gln Arg Glu
    210 215
<210> 70
<211>
217
<212> PRT
<213> Artificial Sequence
<220>
<223> Human feline fusion protein
<400> 70
Ala Ile Gln Leu Thr Gln Ser Pro Ser Ser Leu Ser Ala Ser Val Gly
1 5 10 15
Asp Arg Val Thr Ile Thr Cys Arg Ala Ser Gln Gly Ile Ser Ser Ala
                20 25 30
Leu Ala Trp Tyr Gln Gln Lys Pro Gly Lys Ala Pro Lys Leu Leu Ile
          35 40 45
Tyr Asp Ala Ser Ser Leu Glu Ser Gly Val Pro Ser Arg Phe Ser Gly
    50 55 60
Ser Gly Ser Gly Thr Asp Phe Thr Leu Thr Ile Ser Ser Leu Gln Pro
65 70 75 80
Glu Asp Phe Ala Thr Tyr Tyr Cys Gln Gln Phe Asn Ser Tyr Pro Leu
                  85 90 95
Thr Phe Gly Gly Gly Thr Lys Val Glu Ile Lys Arg Ser Asp Ala Gln
            100 105 110
Pro Ser Val Phe Leu Phe Gln Pro Ser Leu Asp Glu Leu His Thr Gly
          115 120 125
Ser Ala Ser Ile Val Cys Ile Leu Asn Asp Phe Tyr Pro Lys Glu Val
    130 135 140
Asn Val Lys Trp Lys Val Asp Gly Val Val Gln Asn Lys Gly Ile Gln
145 150 155 160
Glu Ser Thr Thr Glu Gln Asn Ser Lys Asp Ser Thr Tyr Ser Leu Ser
                  165 170 175
Ser Thr Leu Thr Met Ser Ser Thr Glu Tyr Gln Ser His Glu Lys Phe
              180 185 190
Ser Cys Glu Val Thr His Lys Ser Leu Ala Ser Thr Leu Val Lys Ser
          195 200 205
Phe Asn Arg Ser Glu Cys Gln Arg Glu
    210 215
```

1. A felinized antibody or antigen binding fragment thereof that binds feline Nerve Growth Factor (NGF), wherein said felinized antibody or antigen binding fragment comprises a heavy chain and a light chain; wherein the light chain comprises three light chain complementary determining regions (CDRs): CDR light 1 (CDRL1), CDR light 2 (CDRL2), and CDR light 3 (CDRL3); and wherein the heavy chain comprises three heavy chain CDRs: CDR heavy 1 (CDRH1), CDR heavy 2 (CDRH2) and CDR heavy 3 (CDRH3); and
(a) wherein CDRH1 comprises the amino acid sequence of SEQ ID NO:1;
(b) wherein CDRH2 comprises the amino acid sequence of SEQ ID NO:2;
(c) wherein CDRH3 comprises the amino acid sequence of SEQ ID NO:3;
(d) wherein CDRL1 comprises the amino acid sequence of SEQ ID NO:4;
(e) wherein CDRL2 comprises the amino acid sequence of SEQ ID NO:5; and
(f) wherein CDRL3 comprises the amino acid sequence of SEQ ID NO:6.

2. The felinized antibody or antigen binding fragment thereof of claim 1, comprising a feline heavy chain constant region;
wherein the feline heavy chain constant region comprises an amino acid sequence having at least 90%, 95%, 98%, or 99% identity, or having 100% identity, with an amino acid sequence selected from the group consisting of SEQ ID NO:54, SEQ ID NO:55, SEQ ID NO:56, or
wherein the feline heavy chain constant region comprises an amino acid sequence having at least 90%, 95%, 98%, or 99% identity with SEQ ID NO:54, and wherein both the aspartic acid residue (D) at position 151 and the asparagine residue (N) at position 183 of the amino acid sequence of SEQ ID NO:54 are substituted by an alanine residue (A), or
wherein the feline heavy chain constant region comprises an amino acid sequence having at least 90%, 95%, 98%, or 99% identity with SEQ ID NO:55, and wherein both the aspartic acid residue (D) at position 151 and the asparagine residue (N) at position 183 of the amino acid sequence of SEQ ID NO:55 are substituted by an alanine residue (A).

3. The felinized antibody or antigen binding fragment thereof of claim 1, wherein the felinized antibody or antigen binding fragment thereof comprises (a) a heavy chain variable region comprising an amino acid sequence selected from the group consisting of SEQ ID NO:57, SEQ ID NO:58, SEQ ID NO:59, and SEQ ID NO:60, (b) a light chain variable region comprising an amino acid sequence selected from the group consisting of SEQ ID NO:61 and SEQ ID NO:62, or (c) both the heavy chain variable region comprising the amino acid sequence selected from the group consisting of SEQ ID NO:57, SEQ ID NO:58, SEQ ID NO:59, and SEQ ID NO:60 and the light chain variable region comprising the amino acid sequence selected from the group consisting of SEQ ID NO:61 and SEQ ID NO: 62.

4. (canceled)

5. The felinized antibody or antigen binding fragment thereof of claim 3, wherein
the heavy chain variable region comprises the amino acid sequence of SEQ ID NO:57 and the light chain variable region comprises the amino acid sequence of SEQ ID NO:61; or
the heavy chain variable region comprises the amino acid sequence of SEQ ID NO:57 and the light chain variable region comprises the amino acid sequence of SEQ ID NO:62; or
the heavy chain variable region comprises the amino acid sequence of SEQ ID NO:58 and the light chain variable region comprises the amino acid sequence of SEQ ID NO:61; or
the heavy chain variable region comprises the amino acid sequence of SEQ ID NO:58 and the light chain variable region comprises the amino acid sequence of SEQ ID NO:62; or
the heavy chain variable region comprises the amino acid sequence of SEQ ID NO:59 and the light chain variable region comprises the amino acid sequence of SEQ ID NO:61; or
the heavy chain variable region comprises the amino acid sequence of SEQ ID NO:59 and the light chain variable region comprises the amino acid sequence of SEQ ID NO:62; or
the heavy chain variable region comprises the amino acid sequence of SEQ ID NO:60 and the light chain variable region comprises the amino acid sequence of SEQ ID NO:61; or
the heavy chain variable region comprises the amino acid sequence of SEQ ID NO:60 and the light chain variable region comprises the amino acid sequence of SEQ ID NO:62.

6. The felinized antibody or antigen binding fragment thereof of claim 1, wherein (a) the light chain comprises an amino acid sequence selected from the group consisting of SEQ ID NO:68 and SEQ ID NO:69, (b) the heavy chain comprises an amino acid sequence selected from the group consisting of SEQ ID NO: 64, SEQ ID NO: 65, SEQ ID NO: 66, and SEQ ID NO: 67, or (c) the light chain comprises an amino acid sequence selected from the group consisting of SEQ ID NO:68 and SEQ ID NO:69 and the heavy chain comprises an amino acid sequence selected from the group consisting of SEQ ID NO: 64, SEQ ID NO: 65, SEQ ID NO: 66, and SEQ ID NO: 67.

7. (canceled)

8. The felinized antibody or antigen binding fragment thereof of claim 6, wherein
the heavy chain comprises the amino acid sequence of SEQ ID NO:64 and the light chain comprises the amino acid sequence of SEQ ID NO:68; or
the heavy chain comprises the amino acid sequence of SEQ ID NO:64 and the light chain comprises the amino acid sequence of SEQ ID NO:69; or
the heavy chain comprises the amino acid sequence of SEQ ID NO:65 and the light chain comprises the amino acid sequence of SEQ ID NO:68; or
the heavy chain comprises the amino acid sequence of SEQ ID NO:65 and the light chain comprises the amino acid sequence of SEQ ID NO:69; or
the heavy chain comprises the amino acid sequence of SEQ ID NO:66 and the light chain comprises the amino acid sequence of SEQ ID NO:68; or
the heavy chain comprises the amino acid sequence of SEQ ID NO:66 and the light chain comprises the amino acid sequence of SEQ ID NO:69; or
the heavy chain comprises the amino acid sequence of SEQ ID NO:67 and the light chain comprises the amino acid sequence of SEQ ID NO:68; or
the heavy chain comprises the amino acid sequence of SEQ ID NO:67 and the light chain comprises the amino acid sequence of SEQ ID NO:69.

9. (canceled)

10. A nucleic acid that encodes
(a) the light chain,
(b) a light chain variable region,
(c) the heavy chain,
(d) a heavy chain variable region,
(e) both the light chain and the heavy chain, or
(f) both a light chain variable region and a heavy chain variable region,
of the felinized antibody or antigen binding fragment thereof claim 1.

11. (canceled)

12. (canceled)

13. An expression vector comprising one or more of the nucleic acids of claim 10.

14. A vector system comprising a pair of the expression vectors of claim 13, wherein
(a) one of the pair of expression vectors encodes the heavy chain of the felinized antibody and the other of the pair of expression vectors encodes the light chain of the felinized antibody; or
(b) one of the pair of expression vectors encodes the heavy chain variable region of the felinized antibody and the other of the pair of expression vectors encodes the light chain variable region of the felinized antibody.

15. A host cell comprising one or more of the expression vector of claim 13.

16. A pair of host cells comprising the vector system of claim 14, wherein one of the pair of host cells comprises the expression vector encoding the heavy chain of the felinized antibody, and the other of the pair of host cells comprises the expression vector encoding the light chain of the felinized antibody.

17. A pharmaceutical composition comprising the felinized antibody or antigen binding fragment thereof of claim 1, and a pharmaceutically acceptable carrier or diluent.

18. A method of aiding in a treatment of a condition associated with pain in a feline comprising administering to the feline a therapeutically effective amount of the pharmaceutical composition of claim 17.

19. The method of claim 18, wherein said condition is osteoarthritis, hyperalgesia, allodynia, pain, or any combination thereof.

20. A method of producing a felinized antibody that binds feline NGF comprising:
a. culturing each one of the pair of host cells of claim 16 in a culture medium either individually or in combination, under conditions wherein the expression vectors are expressed, thereby producing a polypeptide comprising the light chain of the felinized antibody, the heavy chain of the felinized antibody, or both; and
b. recovering the light chain of the felinized antibody, the heavy chain of the felinized antibody, or both from the host cells or culture medium.

21. (canceled)

22. A pharmaceutical composition comprising the vector system of claim 14, and a pharmaceutically acceptable carrier or diluent.

23. A method of aiding in a treatment of a condition associated with pain in a feline comprising administering to the feline a therapeutically effective amount of the pharmaceutical composition of claim 22.

24. The method of claim 23, wherein the condition is osteoarthritis, hyperalgesia, allodynia, pain, or any combination thereof.


SEQUENCE LISTING (70 sequences)

SEQ ID NO: 1 - protein, 5 aa, Homo sapiens
SYSMN

SEQ ID NO: 2 - protein, 17 aa, Homo sapiens
YISRSSHTIFYADSVKG

SEQ ID NO: 3 - protein, 14 aa, Homo sapiens
VYSSGWHVSDYFDY

SEQ ID NO: 4 - protein, 11 aa, Homo sapiens
RASQGISSALA

SEQ ID NO: 5 - protein, 7 aa, Homo sapiens
DASSLES

SEQ ID NO: 6 - protein, 9 aa, Homo sapiens
QQFNSYPLT

SEQ ID NO: 7 - protein, 5 aa, Homo sapiens
ELSIH

SEQ ID NO: 8 - protein, 17 aa, Homo sapiens
GFDPEDGETIYAQKFQG

SEQ ID NO: 9 - protein, 9 aa, Homo sapiens
IGVVTNFDN

SEQ ID NO: 10 - protein, 11 aa, Homo sapiens
RASQAIRNDLG

SEQ ID NO: 11 - protein, 7 aa, Homo sapiens
AAFNLQS

SEQ ID NO: 12 - protein, 9 aa, Homo sapiens
QQYNRYPWT

SEQ ID NO: 13 - protein, 223 aa, Canis lupus
  source 1..223: note = Canis familiaris
EPHPESHVPAGHAIPHAHWTKLQHSLDTALRRARSAPAGAIAARVTGQTRNITVDPKLFKKRRLRSPRVLFSTHPPPVAADAQDLDLEAGSTASVNRTHRSKRSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRA

SEQ ID NO: 14 - protein, 244 aa, synthetic construct
  REGION 1..244: note = modified canine
EPHPESHVPAGHAIPHAHWTKLQHSLDTALRRARSAPAGAIAARVTGQTRNITVDPKLFKKRRLRSPRVLFSTHPPPVAADAQDLDLEAGSTASVNRTHRSKRSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRAHHHHHHGLNDIFEAQKIEWHE

SEQ ID NO: 15 - protein, 796 aa, Canis lupus
  source 1..796: note = Canis familiaris
MLRGGRLGQRGGHGRAAGPGSLLAWLVLASAGAAPCPDVCCPHGPSGLRCTRAGALQSLHRLPGVENLTELYIDNQEHLQHLDAVHLKGLGMLRDLTIVKSGLRSVAPDAFHFTPRLRRLNLSFNALESLSWKTVQGLPLQELVLSGNPLHCSCALHWLLRWEEEGLGGVRGQRLQCPGQGPLALLSNASCGVPVLKVQMPNASVEVGDDVLLQCQVEGQGLERAGWILPEVEELATVTQSGDLPSLGLTLANVTSDLNRKNVTCWAENDVGRAEVSVQVNVSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGDPVEKKDETPFGVSVAVGLAVFACLFLSTLFLALNKCGRRNKFGGNRAVVLAPEDGLAMSLHFMTLGGSSLSPTEGKGSGLQGHIIENPQYFSDACVHHIKRQDIVLKWELGEGAFGKVFLAECHNLLPEQDKMLVAVKALKEVSESARQDFQREAQLLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIECITQGRELERPRACPPEVYAIMRGCWQREPQQRHSIKDVHARLQALAQAPPVYLDVLG

SEQ ID NO: 16 - protein, 455 aa, synthetic construct
  REGION 1..455: note = canine-human fusion protein
EPHPESHVPAGHAIPHAHWTKLQHSLDTALRRARSAPAGAIAARVTGQTRNITVDPKLFKKRRLRSPRVLFSTHPPPVAADAQDLDLEAGSTASVNRTHRSKRSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 629 aa, Canis lupus
  source 1..629: note = Canis familiaris
AAPCPDVCCPHGPSGLRCTRAGALQSLHRLPGVENLTELYIDNQEHLQHLDAVHLKGLGMLRDLTIVKSGLRSVAPDAFHFTPRLRRLNLSFNALESLSWKTVQGLPLQELVLSGNPLHCSCALHWLLRWEEEGLGGVRGQRLQCPGQGPLALLSNASCGVPVLKVQMPNASVEVGDDVLLQCQVEGRGLERAGWILPEVEELATVTQSGDLPSLGLTLANVTSDLNRKNVTCWAENDVGRAEVSVQVNVSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGDPVEKKDETPFGVSVAVGVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 18 - protein, 123 aa, Homo sapiens
EVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSS

SEQ ID NO: 19 - protein, 107 aa, Homo sapiens [sequence omitted]

SEQ ID NO: 20 - protein, 215 aa, synthetic construct
  REGION 1..215: note = modified canine
LGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 21 - protein, 118 aa, Homo sapiens
QVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSS

SEQ ID NO: 22 - protein, 107 aa, Homo sapiens [sequence omitted]

SEQ ID NO: 23 - protein, 458 aa, synthetic construct
  REGION 1..458: note = Human canine fusion protein
EVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 24 - protein, 217 aa, synthetic construct
  REGION 1..217: note = Human canine fusion protein
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 25 - protein, 453 aa, synthetic construct
  REGION 1..453: note = Human canine fusion protein
QVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 26 - protein, 217 aa, synthetic construct
  REGION 1..217: note = Human canine fusion protein
DIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 27 - protein, 123 aa, synthetic construct
  REGION 1..123: note = Caninized human
EVQLVESGGDLVKPGGSLRLSCVASGFTFSSYSMNWIRQAPGKGLQWVSYISRSSHTIFYADSVKGRFTISRDNAKNTLYLQMNSLRDEDTAVYYCARVYSSGWHVSDYFDYWGQGTLVTVSS

SEQ ID NO: 28 - protein, 123 aa, synthetic construct
  REGION 1..123: note = Caninized human
EVQLVESGGDLVKPGGSLRLSCVASGFTLRSYSMNWIRQAPGKGLQWVSYISRSSHTIFYADSVKGRFTISRDNAKNTLYLQMDSLRDEDTAVYYCARVYSSGWHVSDYFDYWGQGILVTVSS

SEQ ID NO: 29 - protein, 107 aa, synthetic construct
  REGION 1..107: note = Caninized human
EIVMTQSPASLSLSQEEKVTITCRASQGISSALAWYQQKPGQAPKLLIYDASSLESGVPSRFSGSGSGTDFSFTISSLEPEDVAVYYCQQFNSYPLTFGQGTKVEIK

SEQ ID NO: 30 - protein, 107 aa, synthetic construct
  REGION 1..107: note = Caninized human
EIQLTQSPASLSLSQEEKVTITCRASQGISSALAWYQQKPGQAPKLLIYDASSLESGVPSRFSGSGSGTDFSLTISSLEPEDFAVYYCQQFNSYPLTFGGGTKVEIK

SEQ ID NO: 31 - protein, 118 aa, synthetic construct
  REGION 1..118: note = Caninized human
EVQLVQSGAEVKKPGASVKVSCKTSGYTFIELSIHWVRQAPGAGLDWMGGFDPEDGETIYAQKFQGRVTLTADTSTSTAYMELSSLRAGDIAVYYCARIGVVTNFDNWGQGTLVTVSS

SEQ ID NO: 32 - protein, 118 aa, synthetic construct
  REGION 1..118: note = Caninized human
EVQLVQSGAEVKKPGASVKVSCKVSGYTLTELSIHWVRQAPGKGLDWMGGFDPEDGETIYAQKFQGRVTLTEDTSTDTAYMELSSLRAGDIAVYYCSTIGVVTNFDNWGQGTLVTVSS

SEQ ID NO: 33 - protein, 107 aa, synthetic construct
  REGION 1..107: note = Caninized human
EIVMTQSPASLSLSQEEKVTITCRASQAIRNDLGWYQQKPGQAPKLLIYAAFNLQSGVPSRFSGSGSGTDFSFTISSLEPEDVAVYYCQQYNRYPWTFGQGTKLEIK

SEQ ID NO: 34 - protein, 107 aa, synthetic construct
  REGION 1..107: note = Caninized human
DIVMTQTPLSLSVSPGETASISCRASQAIRNDLGWFRQKPGQSPQRLIYAAFNLQSGVPDRFSGSGSGTDFTLRISRVEADDTGVYYCQQYNRYPWTFGQGTKLEIK

SEQ ID NO: 35 - protein, 107 aa, synthetic construct
  REGION 1..107: note = caninized human
DIVMTQTPLSLSVSPGETASISCRASQAIRNDLGWFRQKPGKSPKRLIYAAFNLQSGVPDRFSGSGSGTDFTLTISSVEADDTGVYYCQQYNRYPWTFGQGTKLEIK

SEQ ID NO: 36 - protein, 458 aa, synthetic construct
  REGION 1..458: note = Caninized human
EVQLVESGGDLVKPGGSLRLSCVASGFTFSSYSMNWIRQAPGKGLQWVSYISRSSHTIFYADSVKGRFTISRDNAKNTLYLQMNSLRDEDTAVYYCARVYSSGWHVSDYFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 37 - protein, 458 aa, synthetic construct
  REGION 1..458: note = Caninized human
EVQLVESGGDLVKPGGSLRLSCVASGFTLRSYSMNWIRQAPGKGLQWVSYISRSSHTIFYADSVKGRFTISRDNAKNTLYLQMDSLRDEDTAVYYCARVYSSGWHVSDYFDYWGQGILVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 38 - protein, 217 aa, synthetic construct
  REGION 1..217: note = Caninized human
EIVMTQSPASLSLSQEEKVTITCRASQGISSALAWYQQKPGQAPKLLIYDASSLESGVPSRFSGSGSGTDFSFTISSLEPEDVAVYYCQQFNSYPLTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 39 - protein, 217 aa, synthetic construct
  REGION 1..217: note = Caninized human
EIQLTQSPASLSLSQEEKVTITCRASQGISSALAWYQQKPGQAPKLLIYDASSLESGVPSRFSGSGSGTDFSLTISSLEPEDFAVYYCQQFNSYPLTFGGGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 40 - protein, 453 aa, synthetic construct
  REGION 1..453: note = Caninized human
EVQLVQSGAEVKKPGASVKVSCKTSGYTFIELSIHWVRQAPGAGLDWMGGFDPEDGETIYAQKFQGRVTLTADTSTSTAYMELSSLRAGDIAVYYCARIGVVTNFDNWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 41 - protein, 453 aa, synthetic construct
  REGION 1..453: note = Caninized human
EVQLVQSGAEVKKPGASVKVSCKVSGYTLTELSIHWVRQAPGKGLDWMGGFDPEDGETIYAQKFQGRVTLTEDTSTDTAYMELSSLRAGDIAVYYCSTIGVVTNFDNWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 42 - protein, 217 aa, synthetic construct
  REGION 1..217: note = Caninized human
EIVMTQSPASLSLSQEEKVTITCRASQAIRNDLGWYQQKPGQAPKLLIYAAFNLQSGVPSRFSGSGSGTDFSFTISSLEPEDVAVYYCQQYNRYPWTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 43 - protein, 217 aa, synthetic construct
  REGION 1..217: note = Caninized human
DIVMTQTPLSLSVSPGETASISCRASQAIRNDLGWFRQKPGQSPQRLIYAAFNLQSGVPDRFSGSGSGTDFTLRISRVEADDTGVYYCQQYNRYPWTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 44 - protein, 217 aa, synthetic construct
  REGION 1..217: note = Caninized human
DIVMTQTPLSLSVSPGETASISCRASQAIRNDLGWFRQKPGKSPKRLIYAAFNLQSGVPDRFSGSGSGTDFTLTISSVEADDTGVYYCQQYNRYPWTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 45 - protein, 17 aa, Canis lupus
  source 1..17: note = Canis familiaris
FNECRCTDTPPCPVPEP

SEQ ID NO: 46 - protein, 22 aa, Canis lupus
  source 1..22: note = Canis familiaris
PKRENGRVPRPPDCPKCPAPEM

SEQ ID NO: 47 - protein, 20 aa, Canis lupus
  source 1..20: note = Canis familiaris
AKECECKCNCNNCPCPGCGL

SEQ ID NO: 48 - protein, 17 aa, synthetic construct
  REGION 1..17: note = modified canine
PKESTCKCIPPCPVPES

SEQ ID NO: 49 - protein, 216 aa, Canis lupus
  source 1..216: note = Canis familiaris
LGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 50 - protein, 215 aa, Canis lupus
  source 1..215: note = Canis familiaris
LGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 51 - protein, 214 aa, synthetic construct
  REGION 1..214: note = modified canine
LGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 52 - protein, 215 aa, Canis lupus
  source 1..215: note = Canis familiaris
LGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 53 - protein, 216 aa, Canis lupus
  source 1..216: note = Canis familiaris
LGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 54 - protein, 335 aa, Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 55 - protein, 335 aa, Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 56 - protein, 335 aa, Felis catus
ASTTASSVFPLAPSCGTTSGATVALACLVLGYFPEPVTVSWNSGALTSGVHTFPSVLQASGLYSLSSMVTVPSSRWLSDTFTCNVAHRPSSTKVDKTVPKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 57 - protein, 123 aa, synthetic construct
  REGION 1..123: note = Felinized human
DVQLVESGGDLVQPGGSLRLTCAASGFTLRSYSMNWVRQAPGKGLQWVSYISRSSHTIFYADSVKGRFTISRDNAKNTLYLQMNSLKTEDTATYYCAKVYSSGWHVSDYFDYWGQGILVTVSS

SEQ ID NO: 58 - protein, 123 aa, synthetic construct
  REGION 1..123: note = Felinized human
EVQLVESGGDLVQPGGSLRLTCVASGFTFSSYSMNWVRQAPGKGLQWVSYISRSSHTIFYADSVKGRFTISRDNAKNTLYLQMDSLKDEDTATYYCARVYSSGWHVSDYFDYWGQGILVTVSS

SEQ ID NO: 59 - protein, 123 aa, synthetic construct
  REGION 1..123: note = Felinized human
EVQLVESEGDLVQPGGSLRLTCAASGFTLRSYSMNWVRQAPGKGLQWVSYISRSSHTIFYADSVKGRFTISRDNAKNTLYLQMNNLKTEDTATYYCAKVYSSGWHVSDYFDYWGQGILVTVSS

SEQ ID NO: 60 - protein, 123 aa, synthetic construct
  REGION 1..123: note = Felinized human
EVQLVESEGDLVQPGGSLRLTCVASGFTFSSYSMNWVRQAPGKGLQWVSYISRSSHTIFYADSVKGRFTISRDNAKNTLYLQMDSLKDEDTATYYCARVYSSGWHVSDYFDYWGQGILVTVSS

SEQ ID NO: 61 - protein, 107 aa, synthetic construct
  REGION 1..107: note = Felinized human
EIQLTQSPSSLSASPGDRVTITCRASQGISSALAWYQQKPGKVPKLLIYDASSLESGVPSRFSGSGSGTDFTLTISSLEPEDAATYYCQQFNSYPLTFGQGTKLEIK

SEQ ID NO: 62 - protein, 107 aa, synthetic construct
  REGION 1..107: note = Felinized human
AITLTQSPGSLAGSPGQQVTITCRASQGISSALAWYQQKPGQHPKLLIYDASSLESGVPDRFSGSGSGTDFTLTISNLQAEDVASYYCQQFNSYPLTFGQGTKLEIK

SEQ ID NO: 63 - protein, 458 aa, synthetic construct
  REGION 1..458: note = Human-Feline fusion protein
EVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVALGPDDSDVQITWFVDNTQVYTAKTSPREEQFASTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 64 - protein, 458 aa, synthetic construct
  REGION 1..458: note = Felinized human
DVQLVESGGDLVQPGGSLRLTCAASGFTLRSYSMNWVRQAPGKGLQWVSYISRSSHTIFYADSVKGRFTISRDNAKNTLYLQMNSLKTEDTATYYCAKVYSSGWHVSDYFDYWGQGILVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVALGPDDSDVQITWFVDNTQVYTAKTSPREEQFASTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 65 - protein, 458 aa, synthetic construct
  REGION 1..458: note = Felinized human
EVQLVESGGDLVQPGGSLRLTCVASGFTFSSYSMNWVRQAPGKGLQWVSYISRSSHTIFYADSVKGRFTISRDNAKNTLYLQMDSLKDEDTATYYCARVYSSGWHVSDYFDYWGQGILVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVALGPDDSDVQITWFVDNTQVYTAKTSPREEQFASTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 66 - protein, 458 aa, synthetic construct
  REGION 1..458: note = Felinized human
EVQLVESEGDLVQPGGSLRLTCAASGFTLRSYSMNWVRQAPGKGLQWVSYISRSSHTIFYADSVKGRFTISRDNAKNTLYLQMNNLKTEDTATYYCAKVYSSGWHVSDYFDYWGQGILVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVALGPDDSDVQITWFVDNTQVYTAKTSPREEQFASTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 67 - protein, 458 aa, synthetic construct
  REGION 1..458: note = Felinized human
EVQLVESEGDLVQPGGSLRLTCVASGFTFSSYSMNWVRQAPGKGLQWVSYISRSSHTIFYADSVKGRFTISRDNAKNTLYLQMDSLKDEDTATYYCARVYSSGWHVSDYFDYWGQGILVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVALGPDDSDVQITWFVDNTQVYTAKTSPREEQFASTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 68 - protein, 217 aa, synthetic construct
  REGION 1..217: note = Felinized human
EIQLTQSPSSLSASPGDRVTITCRASQGISSALAWYQQKPGKVPKLLIYDASSLESGVPSRFSGSGSGTDFTLTISSLEPEDAATYYCQQFNSYPLTFGQGTKLEIKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 69 - protein, 217 aa, synthetic construct
  REGION 1..217: note = Felinized human
AITLTQSPGSLAGSPGQQVTITCRASQGISSALAWYQQKPGQHPKLLIYDASSLESGVPDRFSGSGSGTDFTLTISNLQAEDVASYYCQQFNSYPLTFGQGTKLEIKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 70 - protein, 217 aa, synthetic construct
  REGION 1..217: note = Human feline fusion protein
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE